(19) United States
(12) Patent Application Publication
(10) Pub. No.: US 2025/0309379 A1
(43) Pub. Date: Oct. 2, 2025

(54) MEDICAL DEVICE BATTERY MANAGEMENT AND USAGE OPTIMIZATION

(71) Applicant: ZOLL Medical Corporation, Chelmsford, MA (US)

(72) Inventors: Gary A. Freeman, Waltham, MA (US); James Lewis, Needham, MA (US); Navid Shaidani, Pelham, NH (US); David Poirier, Belmont, MA (US); Kristin A. Spang, Nashua, NH (US); Mitali J. More, Bedford, MA (US)

(73) Assignee: ZOLL Medical Corporation, Chelmsford, MA (US)

(21) Appl. No.: 19/065,471

(22) Filed: Feb. 27, 2025

Related U.S. Application Data
(60) Provisional application No. 63/571,128, filed on Mar. 28, 2024.

(51) Int. Cl. H01M 10/42; A61N 1/39; G16H 40/63; H01M 10/48

(52) U.S. Cl. CPC H01M 10/4257; G16H 40/63; H01M 10/482; H01M 10/486; H01M 10/488; A61N 1/3975; H01M 2010/4271; H01M 2010/4278; H01M 2220/30

(57) ABSTRACT

Systems and methods are provided for managing, such as mitigating, patient ventilator asynchrony. Example systems include a mechanical ventilation system and at least one controller communicatively coupled with the mechanical ventilation system. The at least one controller obtains input including some or all of: PVA data identifying a detected type of PVA, ventilatory parameter data relating to the ventilation being provided to a patient, a measure of PVA severity relating to the detected type of PVA, and patient data identifying one or more patient characteristics. Based at least in part on the obtained input, the at least one controller determines output including at least one of: one or more PVA mitigation notifications, such as may include root cause information, and at least one adjustment, relating to the mechanical ventilation being provided to the patient, for implementation to mitigate the detected type of PVA.

### CROSS-REFERENCE TO RELATED APPLICATION

This application claims priority to U.S. Prov. App. No. 63/571,128, filed on Mar. 28, 2024, entitled, “MEDICAL DEVICE BATTERY MANAGEMENT AND USAGE OPTIMIZATION.”

### BACKGROUND

Various medical devices, including emergency care medical devices, may be crucially powered by batteries. For example, medical devices such as defibrillators, including external automated defibrillators (AEDs), may rely on battery power to deliver unplanned, urgent, life-saving treatment to a patient. Medical device batteries may remain unused or minimally used, sometimes for long periods of time, but then suddenly become essential in saving a life. In the example case of use with AEDs, with unplanned timing, one or more high-powered defibrillation shocks may be urgently needed, requiring one or several immediate, large power outputs from the AED battery. Should insufficient power be instantly available, or should the battery fail at or between shocks, the patient's life may be lost as a result. In addition, medical device batteries represent a significant cost component associated with medical devices, and their readiness and operation.

However, many factors can affect medical device batteries, and management and optimization of battery usage presents many challenges.

### SUMMARY

One example provide a medical device battery management system for managing a group of commonly owned medical device batteries, comprising: a plurality of medical device batteries, each comprising a processor, a memory and a wireless communication interface, and each configured to write battery health data to the memory and periodically transmit historical battery health data comprising past information relating to battery usage; a medical device database configured to receive and store the historical battery health data for the plurality of medical device batteries; and at least one remote computing device, communicatively coupled with the plurality of medical device batteries and the medical device database, the at least one remote computing device comprising at least one processor and at least one memory, the at least one remote computing device configured to: receive the historical battery health data from the plurality of medical device batteries, update the medical device database with the received historical battery health data, analyze the historical battery health data to (i) identify one or more medical device batteries for which an optimization notification can be provided to recommend an action to optimize battery usage, and to (ii) determine the optimization notification, receive a request from an administrative user relating to a user-defined grouping of medical device batteries to access at least the historical battery health data associated with the user-defined grouping of medical device batteries, and generate and transmit, for display on a display device, summary data relating to the user-defined grouping of medical device batteries, and the determined optimization notification to recommend the action to optimize battery usage.

In some examples, the battery health data includes data relating to at least one of: battery condition, battery status, battery usage, battery error states, battery type, battery manufacturer, battery manufacture date, battery serial number, battery specification information, battery age, battery location, connected device, clinical event data associated with the connected device, battery temperature, battery environment temperature, battery humidity, battery environment humidity, battery motion, battery capacity, battery design capacity, battery remaining capacity, battery power output, battery remaining operating time, battery alarms, battery remaining capacity alarm, battery remaining operating time alarm, battery mode, battery stability, battery reliability, cycle life, and battery calibration. In some examples, the battery health data includes data relating to at least one of: battery wireless communication, battery periodic reporting, and battery periodic software updating. In some examples, the battery health data includes data relating to at least one or: battery charging, battery charging rate, battery absolute state of charge, battery relative state of charge, depth of discharge, battery full depth of discharges.

In some examples, the battery health data includes data relating to at least one of: minimum lifetime battery pack voltage, maximum lifetime battery pack voltage, minimum lifetime battery pack temperature during discharge, maximum lifetime battery pack temperature during discharge, maximum lifetime battery pack temperature during charge, maximum lifetime current during charge, maximum lifetime current during discharge, total discharge amp-hours throughput, total charge amp-hours throughput, energy density, specific energy, nominal capacity, nominal energy, terminal voltage, open-circuit voltage, cut-off voltage, nominal voltage, charge voltage, float voltage, discharge current, C-rate, recommended charge current, specific energy, energy density, specific power, and power density.

In some examples, the battery error states relate to at least one of: battery physical structure, battery configuration, battery hardware, battery software, battery sensors, battery wireless communication interface, battery ports, battery location manager, battery circuit boards, battery RTLS tags, battery RFID tags, battery connected device, a battery voltage fault, a battery pack voltage fault, an under voltage fault, an over voltage fault, a capacity fault, an absolute capacity below limit fault, a temperature fault, an under temperature fault, an over temperature fault, a current fault, a fault of the processor, a fault of the memory, a software fault, a PCB fault, an initial PCB power-on fault, a loss of power to PCB fault, an electronic circuit fault, a computer chip fault, a power-off fault, a reset fault, a clock frequency fault, a watchdog timer reset fault, an alarm fault, a cyclic redundancy check fault, a flash cyclic redundancy check fault, an integrated circuit power supply pins fault, a battery fuel gauge fault, a fuel gauge communication fault, a fuel gauge safety error fault, a VCC supply brownout, a CPU fault, a blown fuse fault, a sensor fault, an initial reconditioning charge cycle required fault, a charge protocol fault, a charge too long fault, a stuck button fault, a call stack overflow fault, a call stack underflow fault, an invalid configuration fault, a software initiated reset fault, a serial flash error fault, an invalid cyclic redundancy check on flash data fault, a microcontroller RAM fault, a battery pack disabled fault, a charge protocol fault, and a software interrupt fault.

In some examples, analyzing the historical battery health data comprises: generating one or more data sets comprising chronologically ordered information relating to each of a plurality of battery parameters over a historical period of time, and analyze the generated data sets in determining the action to be recommended to optimize battery usage. In some examples, analyzing the historical battery health data comprises: determining multiple potential actions, each of which is algorithmically forecasted to improve battery usage, analyzing, and determining a score for, each of the multiple potential actions, wherein the score provides a measure of magnitude of improvement in battery usage forecasted to occur as a result of the potential action, and determine the action to be recommended to optimize battery usage based at least in part on the determined scores. In some examples, the determined optimization notification comprises at least one of: an alert, an alarm, a warning, a message, a message recommending a remedial action, and a message recommending an enhancing action.

In some examples, the at least one remote computing device is configured to implement the recommended action to optimize battery usage upon a user selection to do so. In some examples, each of the plurality of medical device batteries comprises at least one of: at least one motion sensor configured to detect motion of the medical device battery over a period of time, at least one battery temperature sensor configured to detect a temperature of the medical device battery over the period of time, at least one battery environment temperature sensor configured to detect a temperature of an environment of the medical device battery over the period of time, at least one battery humidity sensor configured to detect a humidity level of the medical device battery over the period of time, at least one battery environment humidity sensor configured to detect a humidity level of the environment of the medical device battery over the period of time, and at least one port configured for wired connection to at least one computing or display device.

In some examples, the optimization notification comprises an improvement in battery usage forecasted to result from implementation of the recommended action. In some examples, each of the plurality of medical device batteries comprises a location manager configured to obtain battery location data relating to the medical device battery, wherein the user-defined grouping is based on battery location. In some examples, the at least one computing device is configured to determine the recommended action based at least in part on compliance criteria relating to compliance with battery related, location based legal or regulatory requirements associated with at least one of: devices, data usage, permissions, licensing, reporting, inspection, recall, and error status. In some examples, each of the plurality of medical device batteries is configured to wirelessly transmit the historical battery health data at least one of: automatically according to a specified frequency or protocol, upon a user selection via a physical interface feature or graphical user interface of the battery unit, and upon detection of sufficient proximity with a receiving device for the historical battery health data.

In some examples, the recommended action comprises at least one of: switching locations between at least two medical device batteries, and switching connected devices to be powered by each of the at least two medical device batteries, and wherein determining the recommended action comprises utilizing at least a portion of the past information relating to at three of: battery location, battery environment conditions, battery capacity, battery age, battery error states, battery impedance, battery power output, battery charging, and a connected device to be powered by the medical device battery. In some examples, each of the plurality of medical device batteries comprises a location manager configured to obtain battery location data relating to the medical device battery, wherein the recommended action comprises changing a location of a medical device battery in relation to at least one of: geographic location and in-building related location, and wherein determining the recommended action comprises utilizing at least a portion of the past information relating to at three of: battery location, battery wireless communication settings or protocol, battery motion or shock, battery environment conditions, battery capacity, battery age, battery error states, battery impedance, battery power output, battery charging, a connected device to be powered by the medical device battery.

In some examples, the recommended action comprises replacing a medical device battery, and wherein determining the recommended action comprises utilizing at least a portion of the past information relating to at three of: battery capacity, battery age, battery error states, battery impedance, battery power output, battery charging, and a connected device to be powered by the medical device battery. In some examples, the recommended action comprises changing future battery charging conditions, relative to past battery charging conditions, the battery charging conditions comprising at least three of: battery charge levels during charging, battery temperature during charging, battery environment temperature during charging, battery humidity during charging, battery environment humidity during charging, battery motion or shock during charging, and wherein determining the recommended action comprises utilizing at least a portion of the past information relating to at three of: battery location, battery charge levels, battery temperature, battery environment temperature, battery humidity, battery environment humidity, battery capacity, battery motion or shock, battery capacity, battery impedance, and battery power output.

In some examples, the recommended action comprises reducing battery motion or shock, and wherein determining the recommended action comprises utilizing at least a portion of the past information relating to at three of: battery location, battery motion, battery shock, battery transport, battery impedance, battery error states, battery voltage, battery capacity. In some examples, the recommended action comprises changing a wireless communication related setting or protocol, the wireless communication related setting or protocol relating to at least one of: wireless connection frequency, wireless connection attempt frequency, wireless reporting frequency, software update frequency, wireless communication software protocol, and wireless communication day or time related protocol, and wherein determining the recommended action comprises utilizing at least a portion of the past information relating to at three of: wireless connection frequency, wireless connection attempt frequency, wireless reporting frequency, software update frequency, and wireless communication software protocol, wireless communication day or time related protocol battery location, wireless signal level, battery location, and wireless signal level.

In some examples, the system comprises a mifi device configured to connect to at least one of the plurality of medical device batteries, and wherein the at least one of the plurality of medical device batteries is configured to connect to the mifi device and to use the connection with the mifi device in transmitting the historical battery health data to the at least one remote computing device.

One example provides a medical device battery management system for managing a group of commonly owned medical device batteries, comprising: a plurality of medical device batteries, each comprising a processor, a memory, a wireless communication interface and a location manager, and each configured to write battery health data to the memory and periodically transmit historical battery health data comprising past information relating to battery usage and battery location; a medical device database configured to receive and store the historical battery health data for the plurality of medical device batteries; and at least one remote computing device, communicatively coupled with the plurality of medical device batteries and the medical device database, the at least one remote computing device comprising at least one processor and at least one memory, the at least one remote computing device configured to: receive the historical battery health data from the plurality of medical device batteries, update the medical device database with the received historical battery health data, based on the historical battery health data, (i) identify one or more medical device batteries for which an optimization notification can be provided to recommend an action to optimize battery usage, and to (ii) determine the optimization notification, receive a request from an administrative user relating to a user-defined grouping of medical device batteries to access at least the historical battery health data associated with the user-defined grouping of medical device batteries, and generate and transmit, for display on a display device, summary data relating to the user-defined grouping of medical device batteries, and the determined optimization notification to recommend the action to optimize battery usage.

In some examples, the summary data and the recommended action are displayed on a dashboard that allows a user to implement the recommended action. In some examples, the dashboard comprises a user-interactive map displaying medical device battery locations and medical device battery health status information. In some examples, the user-interactive map comprises a three-dimensional map of at least one building, the three-dimensional map indicating a location of each medical device battery of the plurality of medical device batteries within the at least one building. In some examples, the at least one remote computing device is configured to determine the recommended action based in part on compliance criteria relating to compliance with legal or regulatory requirements associated with multiple regions, each of the multiple regions including a medical device battery of the plurality of medical device batteries, and each of the legal or regulatory requirements relating to at least one of: devices, data usage, permissions, licensing, reporting, inspection, recall, and error status.

In some examples, the at least one computing device is configured to: obtain legal or regulatory requirements data specifying the legal or regulatory requirements, determine the compliance criteria based at least in part on the legal or regulatory requirements data, determine whether the compliance criteria are satisfied, and upon determining that the compliance criteria are not satisfied, determine the recommended action for achieving the compliance.

In some examples, the battery health data includes data relating to at least one of: battery condition, battery status, battery usage, battery error states, battery type, battery manufacturer, battery manufacture date, battery serial number, battery specification information, battery age, battery location, connected device, clinical event data associated with the connected device, battery temperature, battery environment temperature, battery humidity, battery environment humidity, battery motion, battery capacity, battery design capacity, battery remaining capacity, battery power output, battery remaining operating time, battery alarms, battery remaining capacity alarm, battery remaining operating time alarm, battery mode, battery stability, battery reliability, cycle life, and battery calibration.

In some examples, the battery health data includes data relating to at least one of: battery wireless communication, battery periodic reporting, and battery periodic software updating. In some examples, the battery health data includes data relating to at least one or: battery charging, battery charging rate, battery absolute state of charge, battery relative state of charge, depth of discharge, battery full depth of discharges. In some examples, the battery health data includes data relating to at least one of: minimum lifetime battery pack voltage, maximum lifetime battery pack voltage, minimum lifetime battery pack temperature during discharge, maximum lifetime battery pack temperature during discharge, maximum lifetime battery pack temperature during charge, maximum lifetime current during charge, maximum lifetime current during discharge, total discharge amp-hours throughput, total charge amp-hours throughput, energy density, specific energy, nominal capacity, nominal energy, terminal voltage, open-circuit voltage, cut-off voltage, nominal voltage, charge voltage, float voltage, discharge current, C-rate, recommended charge current, specific energy, energy density, specific power, and power density.

In some examples, the battery error states relate to at least one of: battery physical structure, battery configuration, battery hardware, battery software, battery sensors, battery wireless communication interface, battery ports, battery location manager, battery circuit boards, battery RTLS tags, battery RFID tags, battery connected device, a battery voltage fault, a battery pack voltage fault, an under voltage fault, an over voltage fault, a capacity fault, an absolute capacity below limit fault, a temperature fault, an under temperature fault, an over temperature fault, a current fault, a fault of the processor, a fault of the memory, a software fault, a PCB fault, an initial PCB power-on fault, a loss of power to PCB fault, an electronic circuit fault, a computer chip fault, a power-off fault, a reset fault, a clock frequency fault, a watchdog timer reset fault, an alarm fault, a cyclic redundancy check fault, a flash cyclic redundancy check fault, an integrated circuit power supply pins fault, a battery fuel gauge fault, a fuel gauge communication fault, a fuel gauge safety error fault, a VCC supply brownout, a CPU fault, a blown fuse fault, a sensor fault, an initial reconditioning charge cycle required fault, a charge protocol fault, a charge too long fault, a stuck button fault, a call stack overflow fault, a call stack underflow fault, an invalid configuration fault, a software initiated reset fault, a serial flash error fault, an invalid cyclic redundancy check on flash data fault, a microcontroller RAM fault, a battery pack disabled fault, a charge protocol fault, and a software interrupt fault.

In some examples, the at least one remote computing device is configured to analyze the historical battery health data to determine the recommended action. In some examples, the optimization notification comprises an improvement in battery usage forecasted to result from implementation of the recommended action. In some examples, each of the plurality of medical device batteries comprises at least one of: at least one motion sensor configured to detect motion of the medical device battery over a period of time, at least one battery temperature sensor configured to detect a temperature of the medical device battery over the period of time, at least one battery environment temperature sensor configured to detect a temperature of an environment of the medical device battery over the period of time, at least one battery humidity sensor configured to detect a humidity level of the medical device battery over the period of time, at least one battery environment humidity sensor configured to detect a humidity level of the environment of the medical device battery over the period of time, and at least one port configured for wired connection to at least one computing or display device.

In some examples, the recommended action comprises at least one of: switching locations between at least two medical device batteries, and switching connected devices to be powered by each of the at least two medical device batteries, and wherein determining the recommended action comprises utilizing at least a portion of the past information relating to at three of: battery location, battery environment conditions, battery capacity, battery age, battery error states, battery impedance, battery power output, battery charging, and a connected device to be powered by the medical device battery.

In some examples, the recommended action comprises changing a location of a medical device battery in relation to at least one of: geographic location and in-building related location, and wherein determining the recommended action comprises utilizing at least a portion of the past information relating to at three of: battery location, battery wireless communication settings or protocol, battery motion or shock, battery environment conditions, battery capacity, battery age, battery error states, battery impedance, battery power output, battery charging, a connected device to be powered by the medical device battery. In some examples, the recommended action comprises replacing a medical device battery, and wherein determining the recommended action comprises utilizing at least a portion of the past information relating to at three of: battery capacity, battery age, battery error states, battery impedance, battery power output, battery charging, and a connected device to be powered by the medical device battery. In some examples, the recommended action comprises changing future battery charging conditions, relative to past battery charging conditions, the battery charging conditions comprising at least three of: battery charge levels during charging, battery temperature during charging, battery environment temperature during charging, battery humidity during charging, battery environment humidity during charging, battery motion or shock during charging, and wherein determining the recommended action comprises utilizing at least a portion of the past information relating to at three of: battery location, battery charge levels, battery temperature, battery environment temperature, battery humidity, battery environment humidity, battery capacity, battery motion or shock, battery capacity, battery impedance, and battery power output.

In some examples, the recommended action comprises reducing battery motion or shock, and wherein determining the recommended action comprises utilizing at least a portion of the past information relating to at three of: battery location, battery motion, battery shock, battery transport, battery impedance, battery error states, battery voltage, battery capacity. In some examples, the recommended action comprises changing a wireless communication related setting or protocol, the wireless communication related setting or protocol relating to at least one of: wireless connection frequency, wireless connection attempt frequency, wireless reporting frequency, software update frequency, wireless communication software protocol, and wireless communication day or time related protocol, and wherein determining the recommended action comprises utilizing at least a portion of the past information relating to at three of: wireless connection frequency, wireless connection attempt frequency, wireless reporting frequency, software update frequency, and wireless communication software protocol, wireless communication day or time related protocol battery location, wireless signal level, battery location, and wireless signal level.

One example provides a medical device battery management system for managing a group of commonly owned medical device batteries, comprising: a plurality of units, each comprising one or more medical device batteries and a connected device comprising at least one of: a medical device and a dock, the connected device comprising a processor, a memory and a wireless communication interface, wherein: for each of the plurality of units, each of the one or more medical device batteries and the connected device are configured for physical and electrical connection together, and each of the plurality of units is configured to write battery health data to the memory and periodically transmit historical battery health data comprising past information relating to battery usage; a medical device database configured to receive and store the historical battery health data for the plurality of units; and at least one remote computing device, communicatively coupled with the plurality of units and the medical device database, the at least one remote computing device comprising at least one processor and at least one memory, the at least one remote computing device configured to: receive the historical battery health data from the plurality of units, update the medical device database with the received historical battery health data, analyze the historical battery health data to (i) identify one or more medical device batteries for which an optimization notification can be provided to recommend an action to optimize battery usage, and to (ii) determine the optimization notification, receive a request from an administrative user relating to a user-defined grouping of units to access at least the historical battery health data associated with the user-defined grouping of units, and generate and transmit, for display on a display device, summary data relating to the user-defined grouping of units, and the determined optimization notification to recommend the action to optimize battery usage.

In some examples, the at least one remote computing device is configured to analyze the historical battery health data to determine the recommended action. In some examples, each of the one or more units is configured such that: the one or more medical device batteries are configured to include one or more electrical contacts configured for electrical connection with the connected device, and the connected device is configured to include one or more electrical contacts configured for electrical connection with the one or more medical device batteries. In some examples, for each of the one or more units, the connected device comprises a defibrillator, and one or more medical device batteries comprise one or more defibrillator batteries. In some examples, for each of the one or more units, the defibrillator is configured to obtain the historical battery health data and cause the historical battery health data to be transmitted to be received by the at least one computing device, and wherein the historical battery health data comprises battery usage data associated with defibrillation shocks provided by the defibrillator. In some examples, for each of the plurality of units, the connected device comprises a dock configured to physically receive the one or more medical device batteries.

In some examples, for each of the plurality of units, the dock is configured to obtain the historical battery health data, to transmit the historical battery health data to be received by the at least one computing device, and to charge the one or more medical device batteries. In some examples, for each of the one or more units, the one or more medical device batteries comprise a plurality of medical device batteries, and wherein the dock is configured to provide protective storage for the plurality of medical device batteries. In some examples, each of the plurality of units comprises at least one of: at least one motion sensor configured to detect motion of the medical device battery over a period of time, at least one battery temperature sensor configured to detect a temperature of the medical device battery over the period of time, at least one battery environment temperature sensor configured to detect a temperature of an environment of the medical device battery over the period of time, at least one battery humidity sensor configured to detect a humidity level of the medical device battery over the period of time, at least one battery environment humidity sensor configured to detect a humidity level of the environment of the medical device battery over the period of time, and at least one port configured for wired connection to at least one computing or display device. In some examples, the optimization notification comprises an improvement in battery usage forecasted to result from implementation of the recommended action.

In some examples, the battery health data includes data relating to at least one of: battery condition, battery status, battery usage, battery error states, battery type, battery manufacturer, battery manufacture date, battery serial number, battery specification information, battery age, battery location, connected device, clinical event data associated with the connected device, battery temperature, battery environment temperature, battery humidity, battery environment humidity, battery motion, battery capacity, battery design capacity, battery remaining capacity, battery power output, battery remaining operating time, battery alarms, battery remaining capacity alarm, battery remaining operating time alarm, battery mode, battery stability, battery reliability, cycle life, and battery calibration. In some examples, the battery health data includes data relating to at least one of: battery wireless communication, battery periodic reporting, and battery periodic software updating. In some examples, the battery health data includes data relating to at least one or: battery charging, battery charging rate, battery absolute state of charge, battery relative state of charge, depth of discharge, battery full depth of discharges.

In some examples, the battery health data includes data relating to at least one of: minimum lifetime battery pack voltage, maximum lifetime battery pack voltage, minimum lifetime battery pack temperature during discharge, maximum lifetime battery pack temperature during discharge, maximum lifetime battery pack temperature during charge, maximum lifetime current during charge, maximum lifetime current during discharge, total discharge amp-hours throughput, total charge amp-hours throughput, energy density, specific energy, nominal capacity, nominal energy, terminal voltage, open-circuit voltage, cut-off voltage, nominal voltage, charge voltage, float voltage, discharge current, C-rate, recommended charge current, specific energy, energy density, specific power, and power density.

In some examples, the battery error states relate to at least one of: battery physical structure, battery configuration, battery hardware, battery software, battery sensors, battery wireless communication interface, battery ports, battery location manager, battery circuit boards, battery RTLS tags, battery RFID tags, battery connected device, a battery voltage fault, a battery pack voltage fault, an under voltage fault, an over voltage fault, a capacity fault, an absolute capacity below limit fault, a temperature fault, an under temperature fault, an over temperature fault, a current fault, a fault of the processor, a fault of the memory, a software fault, a PCB fault, an initial PCB power-on fault, a loss of power to PCB fault, an electronic circuit fault, a computer chip fault, a power-off fault, a reset fault, a clock frequency fault, a watchdog timer reset fault, an alarm fault, a cyclic redundancy check fault, a flash cyclic redundancy check fault, an integrated circuit power supply pins fault, a battery fuel gauge fault, a fuel gauge communication fault, a fuel gauge safety error fault, a VCC supply brownout, a CPU fault, a blown fuse fault, a sensor fault, an initial reconditioning charge cycle required fault, a charge protocol fault, a charge too long fault, a stuck button fault, a call stack overflow fault, a call stack underflow fault, an invalid configuration fault, a software initiated reset fault, a serial flash error fault, an invalid cyclic redundancy check on flash data fault, a microcontroller RAM fault, a battery pack disabled fault, a charge protocol fault, and a software interrupt fault.

In some examples, each of the plurality of units comprises a location manager configured to obtain battery location data relating to the medical device battery, wherein the user-defined grouping is based on battery location. In some examples, the at least one computing device is configured to determine the recommended action based at least in part on compliance criteria relating to compliance with battery related, location based legal or regulatory requirements associated with at least one of: devices, data usage, permissions, licensing, reporting, inspection, recall, and error status. In some examples, the recommended action comprises at least one of: switching locations between at least two medical device batteries, and switching connected devices to be powered by each of the at least two medical device batteries, and wherein determining the recommended action comprises utilizing at least a portion of the past information relating to at three of: battery location, battery environment conditions, battery capacity, battery age, battery error states, battery impedance, battery power output, battery charging, and a connected device to be powered by the medical device battery.

In some examples, each of the plurality of medical device batteries comprises a location manager configured to obtain battery location data relating to the medical device battery, wherein the recommended action comprises changing a location of a medical device battery in relation to at least one of: geographic location and in-building related location, and wherein determining the recommended action comprises utilizing at least a portion of the past information relating to at three of: battery location, battery wireless communication settings or protocol, battery motion or shock, battery environment conditions, battery capacity, battery age, battery error states, battery impedance, battery power output, battery charging, a connected device to be powered by the medical device battery. In some examples, the recommended action comprises replacing a medical device battery, and wherein determining the recommended action comprises utilizing at least a portion of the past information relating to at three of: battery capacity, battery age, battery error states, battery impedance, battery power output, battery charging, and a connected device to be powered by the medical device battery.

In some examples, the recommended action comprises changing future battery charging conditions, relative to past battery charging conditions, the battery charging conditions comprising at least three of: battery charge levels during charging, battery temperature during charging, battery environment temperature during charging, battery humidity during charging, battery environment humidity during charging, battery motion or shock during charging, and wherein determining the recommended action comprises utilizing at least a portion of the past information relating to at three of: battery location, battery charge levels, battery temperature, battery environment temperature, battery humidity, battery environment humidity, battery capacity, battery motion or shock, battery capacity, battery impedance, and battery power output. In some examples, the recommended action comprises reducing battery motion or shock, and wherein determining the recommended action comprises utilizing at least a portion of the past information relating to at three of: battery location, battery motion, battery shock, battery transport, battery impedance, battery error states, battery voltage, battery capacity.

In some examples, the recommended action comprises changing a wireless communication related setting or protocol, the wireless communication related setting or protocol relating to at least one of: wireless connection frequency, wireless connection attempt frequency, wireless reporting frequency, software update frequency, wireless communication software protocol, and wireless communication day or time related protocol, and wherein determining the recommended action comprises utilizing at least a portion of the past information relating to at three of: wireless connection frequency, wireless connection attempt frequency, wireless reporting frequency, software update frequency, and wireless communication software protocol, wireless communication day or time related protocol battery location, wireless signal level, battery location, and wireless signal level.

One example provides a medical device battery management system for managing a group of commonly owned medical device batteries, comprising: a plurality of units, each comprising one or more medical device batteries and a connected device comprising at least one of: a medical device and a dock, the connected device comprising a processor, a memory a wireless communication interface and a location manager, wherein: for each of the plurality of units, each of the one or more medical device batteries and the connected device are configured for physical and electrical connection together, and each of the plurality of units is configured to write battery health data to the memory and periodically transmit historical battery health data comprising past information relating to battery usage and battery location; a medical device database configured to receive and store the historical battery health data for the plurality of units; and at least one remote computing device, communicatively coupled with the plurality of units and the medical device database, the at least one remote computing device comprising at least one processor and at least one memory, configured to: receive the historical battery health data from the plurality of units, update the medical device database with the received historical battery health data, based on the historical battery health data to (i) identify one or more medical device batteries for which an optimization notification can be provided to recommend an action to optimize battery usage, and to (ii) determine the optimization notification, receive a request from an administrative user relating to a user-defined grouping of units to access at least the historical battery health data associated with the user-defined grouping of units, and generate and transmit, for display on a display device, summary data relating to the user-defined grouping of units, and the determined optimization notification to recommend the action to optimize battery usage.

In some examples, the at least one remote computing device is configured to analyze the historical battery health information to determine the recommended action. In some examples, each of the one or more units is configured such that: the one or more medical device batteries are configured to include one or more electrical contacts configured for electrical connection with the connected device, and the connected device is configured to include one or more electrical contacts configured for electrical connection with the one or more medical device batteries. In some examples, for each of the one or more units, the connected device comprises a defibrillator, and one or more medical device batteries comprise one or more defibrillator batteries. In some examples, for each of the one or more units, the defibrillator is configured to obtain the historical battery health data and cause the historical battery health data to be transmitted to be received by the at least one computing device, and wherein the historical battery health data comprises battery usage data associated with defibrillation shocks provided by the defibrillator.

In some examples, for each of the plurality of units, the connected device comprises a dock configured to physically receive the one or more medical device batteries. In some examples, for each of the plurality of units, the dock is configured to obtain the historical battery health data, to transmit the historical battery health data to be received by the at least one computing device, and to charge the one or more medical device batteries. In some examples, for each of the one or more units, the one or more medical device batteries comprise a plurality of medical device batteries, and wherein the dock is configured to provide protective storage for the plurality of medical device batteries.

In some examples, each of the plurality units comprises at least one of: at least one motion sensor configured to detect motion of the medical device battery over a period of time, at least one battery temperature sensor configured to detect a temperature of the medical device battery over the period of time, at least one battery environment temperature sensor configured to detect a temperature of an environment of the medical device battery over the period of time, at least one battery humidity sensor configured to detect a humidity level of the medical device battery over the period of time, at least one battery environment humidity sensor configured to detect a humidity level of the environment of the medical device battery over the period of time, and at least one port configured for wired connection to at least one computing or display device.

In some examples, the battery health data includes data relating to at least one of: battery condition, battery status, battery usage, battery error states, battery type, battery manufacturer, battery manufacture date, battery serial number, battery specification information, battery age, battery location, connected device, clinical event data associated with the connected device, battery temperature, battery environment temperature, battery humidity, battery environment humidity, battery motion, battery capacity, battery design capacity, battery remaining capacity, battery power output, battery remaining operating time, battery alarms, battery remaining capacity alarm, battery remaining operating time alarm, battery mode, battery stability, battery reliability, cycle life, and battery calibration.

In some examples, the battery health data includes data relating to at least one of: battery wireless communication, battery periodic reporting, and battery periodic software updating. In some examples, the battery health data includes data relating to at least one or: battery charging, battery charging rate, battery absolute state of charge, battery relative state of charge, depth of discharge, battery full depth of discharges. In some examples, the battery health data includes data relating to at least one of: minimum lifetime battery pack voltage, maximum lifetime battery pack voltage, minimum lifetime battery pack temperature during discharge, maximum lifetime battery pack temperature during discharge, maximum lifetime battery pack temperature during charge, maximum lifetime current during charge, maximum lifetime current during discharge, total discharge amp-hours throughput, total charge amp-hours throughput, energy density, specific energy, nominal capacity, nominal energy, terminal voltage, open-circuit voltage, cut-off voltage, nominal voltage, charge voltage, float voltage, discharge current, C-rate, recommended charge current, specific energy, energy density, specific power, and power density.

In some examples, the battery error states relate to at least one of: battery physical structure, battery configuration, battery hardware, battery software, battery sensors, battery wireless communication interface, battery ports, battery location manager, battery circuit boards, battery RTLS tags, battery RFID tags, battery connected device, battery chemistry, battery mechanics, battery computerized components, battery electrical components, a battery voltage fault, a battery pack voltage fault, an under voltage fault, an over voltage fault, a capacity fault, an absolute capacity below limit fault, a temperature fault, an under temperature fault, an over temperature fault, a current fault, a fault of the processor, a fault of the memory, a software fault, a PCB fault, an initial PCB power-on fault, a loss of power to PCB fault, an electronic circuit fault, a computer chip fault, a power-off fault, a reset fault, a clock frequency fault, a watchdog timer reset fault, an alarm fault, a cyclic redundancy check fault, a flash cyclic redundancy check fault, an integrated circuit power supply pins fault, a battery fuel gauge fault, a fuel gauge communication fault, a fuel gauge safety error fault, a VCC supply brownout, a CPU fault, a blown fuse fault a sensor fault, an initial reconditioning charge cycle required fault, a charge protocol fault, a charge too long fault, a stuck button fault, a call stack overflow fault, a call stack underflow fault, an invalid configuration fault, a software initiated reset fault, a serial flash error fault, an invalid cyclic redundancy check on flash data fault, a microcontroller RAM fault, a battery pack disabled fault, a charge protocol fault, and a software interrupt fault.

In some examples, the summary data and the recommended action are displayed on a dashboard that allows a user to implement the recommended action. In some examples, the dashboard comprises a user-interactive map displaying medical device battery locations and medical device battery health status information. In some examples, the user-interactive map comprises a three-dimensional map of at least one building, the three-dimensional map indicating a location of each medical device battery, of the plurality of units, within the at least one building. In some examples, the at least one computing device is configured to determine the recommended action based at least in part on compliance criteria relating to compliance with battery related, location based legal or regulatory requirements associated with at least one of: devices, data usage, permissions, licensing, reporting, inspection, recall, and error status.

In some examples, the at least one computing device is configured to: obtain legal or regulatory requirements data specifying the legal or regulatory requirements, determine the compliance criteria based at least in part on the legal or regulatory requirements data, determine whether the compliance criteria are satisfied, and upon determining that the compliance criteria are not satisfied, determine the recommended action for achieving the compliance. In some examples, the recommended action comprises at least one of: switching locations between at least two medical device batteries, and switching connected devices to be powered by each of the at least two medical device batteries, and wherein determining the recommended action comprises utilizing at least a portion of the past information relating to at three of: battery location, battery environment conditions, battery capacity, battery age, battery error states, battery impedance, battery power output, battery charging, and a connected device to be powered by the medical device battery.

In some examples, the recommended action comprises changing a location of a medical device battery in relation to at least one of: geographic location and in-building related location, and wherein determining the recommended action comprises utilizing at least a portion of the past information relating to at three of: battery location, battery wireless communication settings or protocol, battery motion or shock, battery environment conditions, battery capacity, battery age, battery error states, battery impedance, battery power output, battery charging, a connected device to be powered by the medical device battery. In some examples, the recommended action comprises replacing a medical device battery, and wherein determining the recommended action comprises utilizing at least a portion of the past information relating to at three of: battery capacity, battery age, battery error states, battery impedance, battery power output, battery charging, and a connected device to be powered by the medical device battery.

In some examples, the recommended action comprises changing future battery charging conditions, relative to past battery charging conditions, the battery charging conditions comprising at least three of: battery charge levels during charging, battery temperature during charging, battery environment temperature during charging, battery humidity during charging, battery environment humidity during charging, battery motion or shock during charging, and wherein determining the recommended action comprises utilizing at least a portion of the past information relating to at three of: battery location, battery charge levels, battery temperature, battery environment temperature, battery humidity, battery environment humidity, battery capacity, battery motion or shock, battery capacity, battery impedance, and battery power output.

In some examples, the recommended action comprises reducing battery motion or shock, and wherein determining the recommended action comprises utilizing at least a portion of the past information relating to at three of: battery location, battery motion, battery shock, battery transport, battery impedance, battery error states, battery voltage, battery capacity. In some examples, the recommended action comprises changing a wireless communication related setting or protocol, the wireless communication related setting or protocol relating to at least one of: wireless connection frequency, wireless connection attempt frequency, wireless reporting frequency, software update frequency, wireless communication software protocol, and wireless communication day or time related protocol, and wherein determining the recommended action comprises utilizing at least a portion of the past information relating to at three of: wireless connection frequency, wireless connection attempt frequency, wireless reporting frequency, software update frequency, and wireless communication software protocol, wireless communication day or time related protocol battery location, wireless signal level, battery location, and wireless signal level.

### BRIEF DESCRIPTION OF THE DRAWINGS

Various aspects of embodiments of the present disclosure are discussed below with reference to the accompanying figures, which are not intended to be drawn to scale. The figures are included for illustrative purposes and a further understanding of the various aspects and examples. The figures are incorporated in and constitute a part of this specification, but are not intended to limit the scope of the disclosure. In the figures, identical or nearly identical components that are illustrated in various figures may be represented by like numerals. For purposes of clarity, not every component may be labeled in every figure.

FIG. 1 is a block diagram illustrating an example medical device battery management and optimization system, including a group of medical device batteries.

FIG. 2 is a block diagram illustrating an example medical device battery management and optimization system, including a group of units, each including an AED and an AED battery.

FIG. 3 is a block diagram illustrating an example medical device battery management and optimization system, including a group of units, each including a medical device battery dock and medical device batteries.

FIG. 4 is a block diagram illustrating components of example medical device batteries and units.

FIG. 5 includes a flow diagram according to an example medical device battery management and optimization method.

FIG. 6 is a block diagram illustrating an example medical device battery management and optimization analysis engine.

FIG. 7 is a block diagram illustrating example types of battery health data, which may include historical battery health data.

FIG. 8 is a block diagram illustrating example aspects of output of a medical device battery management and optimization system.

FIG. 9A-B are flow diagrams according to example medical device battery management and optimization methods.

FIG. 10 is a mixed diagram illustrating examples aspects of medical device management and optimization system analysis, including generation and use of historical battery parameter plots.

FIG. 11 illustrates an example battery management dashboard including a location map and a battery related user action menu.

FIG. 12 illustrates example portions of a battery management dashboard including battery health related information and a battery usage optimization notification/recommendation.

FIG. 13 illustrates example portions of a battery management dashboard including a display representing a three-dimensional map including icons representing batteries.

FIG. 14A illustrates an example battery management dashboard including device readiness information and battery related information.

FIG. 14B illustrates an example battery management dashboard including device readiness and battery health information for a group of devices.

FIG. 15 illustrates an example battery management dashboard including display indicators for each of two batteries, along with a battery usage optimization notification/recommendation relating to switching of battery locations.

FIG. 16 illustrates an example battery management dashboard including display indicators for each of two AED batteries, along with a battery usage optimization notification/recommendation relating to switching of AEDs associated with batteries.

FIG. 17 illustrates an example battery management dashboard including a battery usage optimization notification/recommendation relating to changing a storage location for a battery.

FIG. 18 illustrates an example battery management dashboard including a battery usage optimization notification/recommendation relating to replacing a battery with a new battery.

FIG. 19 illustrates an example battery management dashboard including a battery usage optimization notification/recommendation relating to switching medical devices associated with each of two batteries and reducing charging temperature associated with a battery.

FIG. 20 illustrates an example battery management dashboard including a battery usage optimization notification/recommendation relating to reducing battery motion or shock.

FIG. 21 illustrates an example battery management dashboard including a battery usage optimization notification/recommendation relating to adjusting wireless communication settings.

FIG. 22A, B and C include, in association with a medical device battery compliance engine, respectively, a block diagram illustrating an example associated regional structure, a flow diagram illustrating an example method, and an example compliance related dashboard including an associated map.

FIGS. 23A-C illustrate various examples of proximity based triggering of transmission and display of battery health data from a medical device battery or unit.

FIG. 24 is an example environment including wifi-enabled devices, including batteries and units, and a mifi device including a battery management program.

FIG. 25 is a block diagram illustrating example components of various devices described with reference to preceding figures.

### DETAILED DESCRIPTION

Effective management and optimization of usage of, for example, a group of medical device batteries, poses many challenges. Medical device batteries, such as, for example, lithium-ion batteries, may be needed, sometimes after a significant, unpredictable period of non-use or minimal use, to crucially provide power, sometimes in large amounts, for potentially life-saving medical devices. Such medical devices may include, for example, defibrillators, which may include Automated External Defibrillators (AEDs), ventilators, such as portable or field ventilators, and various others. Such batteries may have an initial, or absolute, capacity, which may be measured or characterized, for example, in units of ampere-hours (Ah) or watt-hours (Wh). The absolute capacity may represent the total amount of electrical energy that can be generated by the battery when the battery is new. However, due to a large variety of factors and patterns, over time, a battery's capacity may decrease over time, although at a wide range of possible rates. As the battery's capacity decreases, even when fully charged, the battery can no longer deliver 100% of the energy that it could when it was new. Additionally, over time and due to a variety of factors (e.g., excessive shock/motion or high battery temperature), a battery's stability may be compromised, which may lead to an increased risk of battery error or failure.

Given the critical role, and considerable cost, of medical device batteries, together with the complexities of the many factors and patterns and aspects of usage that, over time, contribute to various aspects of battery health, management and optimization of battery usage poses significant challenges. Furthermore, management and optimization may be compromised if insufficient battery health data is obtained and used, or used ineffectively. In some embodiments, for example, historical battery health data is used in determining aspects of usage over time with regard to particular battery parameters (e.g., power output, charging, capacity decline rates, stability changes, environmental condition changes, and many others). Additionally, collection of such data for a spectrum of battery parameters, which may include historical battery health data, and effective analysis thereof, may enable determination and providing of a variety of battery usage optimization notifications/recommendations, which may in turn lead to more effective battery usage management and optimization. As such, some embodiments provide technical solutions to technical problems associated with medical device battery management and optimization, including in areas associated with optimal battery health data collection and optimal analysis of such data to determine a battery usage optimization notification/recommendation (e.g., an optimization notification to recommend an action to optimize battery usage).

In some embodiments, effectively obtaining and utilizing historical battery health data, such as for a variety of battery parameters, for each of a group of potentially geographically distributed batteries, may allow for effective, centralized management and optimization of battery usage. Some embodiments utilize analysis of the historical battery health data in determining an effective recommended action for optimizing future battery usage for one or more batteries of a group (e.g., the recommended action could include exchange/swapping of locations of two or more batteries, changing the location of a battery, replacing a battery, changing battery charging conditions, reducing in battery motion or shock, changing of a battery wireless communication setting, or many others). In some embodiments, current battery health data is also used.

In some embodiments, analysis of the collected historical battery health data (potentially in addition to other data) may be used to generate chronologically ordered sets of data, such as for each of multiple battery parameters, or for groups of multiple battery parameters. For example, the chronologically ordered sets of data may include chronologically tracked patterns of data, such as plots over time (whether, e.g., for display or generated internally by a computing device(s) for analysis). The generated plots may be analyzed, such as using various algorithms, e.g., regression algorithms, mathematical models, or artificial intelligence models, to determine patterns over time associated with battery usage, including patterns relating to multiple battery parameters as they may change over time. The analysis may also include forecasting of potential outcomes or results of specific potential changes in specific battery usage parameters. This analysis, including forecasting, may be used in determining a specific action for recommendation to optimize battery usage. Furthermore, in some embodiments, the analysis may include scoring each of multiple potential recommended actions, where each score provides a measure of a magnitude of improvement in battery usage forecasted to result from the recommended change. The scoring may be based on many factors, or a combination of weighted factors, for example.

In some embodiments, a commonly owned group, or fleet, of medical device batteries may be managed centrally, such as by an administrative user. A display, such as a graphical user interface (GUI) based display, such as a dashboard, may be provided, such as for tracking, obtaining information about, managing, and optimizing the use of one or more batteries of the fleet of medical device batteries. For example, via the dashboard, the administrative user may request and obtain information on a specific group of medical device batteries. The group may be, for example, those medical device batteries currently located in a specified user-defined region, such as a geographic region, or localized region, such as within a building in which multiple batteries are located. The obtained, displayed information may include, for example, summary data (e.g., associated with battery health) relating to the group of batteries, or one or more of them. In some embodiments, a display may include a map of a region, or a map relating to a three dimensional building space, that may show the locations of each of multiple batteries, along with information about each. Additionally, in some embodiments, an optimization notification may be provided, which may provide a recommended action to optimize battery usage for one or more batteries of the group. Furthermore, the optimization notification may include additional information, such as may relate to the recommended action, such as a specific improvement forecasted to occur if the change is implemented (e.g., a specific projected increase in remaining battery lifespan). For example, estimated remaining battery lifespan may refer to an estimated amount of time into the future during which a medical device battery is forecasted to remain viable before needing to be replaced, such as by continuing to have sufficient remaining capacity to power an anticipated connected medical device (e.g., an AED) for its intended use.

In some embodiments, a variety of data is used in an analysis to determine a recommended action to optimize battery usage, including historical battery health data that may relate to various battery parameters, and which may include current battery health data. Furthermore, in some embodiments, the historical battery health data may be obtained and stored, and periodically transmitted, such as using wireless communication, and such as by the batteries themselves, or by devices to which the batteries may be connected as units. In some embodiments, the periodically transmitted historical battery health data may be received by one or more remote computing devices, such as a central server, and stored in a medical device database, which may include a database in which, e.g., data associated with medical devices or medical device batteries is stored. In some embodiments, recommended actions may include actions that substantially increase the remaining lifespan of a medical device battery (e.g., by recommending changing battery location to reduce battery temperature or usage level, or by recommending changing, replacing, or performing maintenance on a component of a battery or unit, such as fuse or sensor, for example), or achieve load balancing for multiple batteries. Furthermore, in some embodiments, historical battery health data may be analyzed to determine that certain error states are likely to occur soon (e.g., a battery component that may soon fail), and notifications/recommendations may include warnings or alerts to service or condition such batteries or replace such components to avoid battery failure or error states, for example.

In some embodiments, individual medical device batteries or connected devices (e.g., a medical device, such as an AED, or a dock that may, e.g., house and charge multiple batteries) may include various hardware and software, which may, e.g., allow or enhance the ability to obtain, store, and transmit various types of historical battery health data. This may include, for example, a processor, a memory, a wireless communication interface, a location manager (which may include, e.g., a GPS receiver), a motion sensor (e.g., an accelerometer), battery and/or battery environment temperature and humidity sensors, various electrical and/or physical connection ports, and proximity sensing/locator components (e.g., for facilitating communication with a local receiving device).

In some embodiments, optimizing battery usage may include changing or adjusting one or more battery or other device parameters to comply, or optimally comply, with potentially multiple applicable sets of legal or regulatory rules associated with a medical device battery, or an associated connected device, or its usage (e.g., wireless communication related requirements/ranges). In some cases, regional requirements may apply for each of several regions that may include the battery's or other device's location, such as state, county and city requirements, which may not necessarily specify identical compliant requirements/ranges (e.g., they may specify different, though overlapping, compliance ranges). In some embodiments, these various regional (or other) rules may be collected or proactively imported by a remote computing device, such as a central server, stored in a medical device database, and analyzed to determine appropriate battery usage optimization recommendations. For example, the analysis may be used to determine a battery usage related setting range (e.g., relating to a frequency of wireless connection, or to wireless communication software protocol) that complies with each of multiple different but overlapping required ranges. The analysis may be further used to determine, e.g., an optimal battery usage related setting within that range, and to determine a recommended action to adjust the setting accordingly, in order to achieve compliance with all requirements, or to achieve an optimal setting within a compliant setting range, for example.

In some embodiments, devices such as medical device batteries or units that may include medical device batteries (e.g., defibrillators, AEDs, battery docking stations), may include, or be connected with, one or more sensors or accessories. The sensors or accessories may allow sensing of various conditions, including environmental conditions, such as location, temperature, humidity, pressure, and other conditions, as described herein. In some embodiments, information regarding the sensed conditions may be stored in a memory of the device and/or elsewhere (e.g., a remote computing device or system and/or a centralized database), and may be chronologically ordered to allow analysis of patterns, making of predictions, etc. The information may be analyzed and used, for example, in determining notifications, recommendations and/or adjustment as relates to medical device battery management, such as for optimization relating to the medical device batteries and their usage or operation. In some embodiments, for example, based on such information, or patterns determined from such information, (e.g., patterns relating to location, temperature, humidity, pressure, etc.), adjustments or recommendations may be determined as relates to, for example, change of storage location or conditions, or change of one or more operational aspects of the battery or unit, many of which are described herein (e.g., wireless connection frequency). For example, in some embodiments, notifications, recommendations and/or adjustments may be determined at a remote computing system, which may then communicate the notifications, recommendations or adjustments to a user or service provider of a battery or unit (e.g., to change or switch storage locations, to change storage conditions, to change uses, etc.), or may communicate adjustments to the battery or unit to change and optimize operational aspects of the battery and/or unit (e.g., wireless connection aspects, charging times or conditions, etc.).

For example, defibrillation electrodes may have limited shelf life or an expiration date, since they may include a conductive gel that may dry out over time. In some embodiments, recommendations may be determined that relate to AED servicing or electrode replacement. For example, in some embodiments, electrode replacement may be recommended sooner if battery storage environmental conditions (e.g., high temperature and/or low humidity) may speed drying out of the conductive gel, or may be recommended later under the other conditions (e.g., lower temperature and/or higher humidity).

Furthermore, in some embodiments, medical device batteries or units that may include medical device batteries (e.g., defibrillators, AEDs, battery docking stations), may include one or more ports and/or accessories that may facilitate environmental condition sensing, such as by allowing connection of sensors. For example, in some embodiments, one or more sensors (e.g., temperature, humidity or pressure sensors) may be connected to one or more ports of the battery or unit (e.g., a USB port, universal port, or data interface (DI) port, for example). Furthermore, in some embodiments, one or more accessories may be used to facilitate connection of the sensors. For example, a dongle or other facilitating or connecting device may be inserted into a port of the battery or unit (e.g., AED), and may be used to connect one or more of the sensors to the battery or unit (e.g., one end of the dongle may connect to the battery or unit and the other end of the dongle may connect to the sensor or accessory).

Medical device batteries, while often critical to availability and successful use of potentially life-saving medical devices, can be difficult track, manage, use efficiently. For example, a wide range of battery health information, including chronologically ordered or orderable historical battery health information, can be difficult to obtain and collect, including potentially from many batteries at different locations. Yet, this information can be critical to tracking battery parameters and usage. Some embodiments allow or enhance the ability to efficiently or easily obtain and collect this information or a greater amount or variety of this information, and to use or analyze the collected information to determine recommendations for actions, and/or to implement actions, that may optimize battery usage for one or several medical device batteries. For example, in some embodiments, medical device batteries, or units including medical device batteries, include hardware and software to allow collection of various battery health information, including historical battery health information, and to determine and provide recommendations for optimization of battery usage accordingly. For example, batteries or units may include various types of sensors, location managers/GPS receivers, and wireless communication interfaces to allow for collection of a wide range of battery health information over time. Furthermore, batteries or units may include processors and memories, along with software, to allow collection and storage of battery health information over time, and to wirelessly transmit such information (e.g., by medical device batteries or connected devices such as defibrillators or docks), e.g., for central storage in a medical device battery database, and for analysis to determine recommendations or actions to optimize battery usage, such as may include adjustments to battery usage parameters or settings, for example.

Furthermore, some embodiments enable or enhance tracking of medical device batteries. For example, batteries or units may include wired and wireless communication interfaces and ports to facilitate efficient communication of battery health information, both locally (e.g., via wired connection to a collection or display device, or wirelessly, which could include use of a mifi device) and remotely (e.g., via wireless communication to a remote computing device and storage in a database). Furthermore, batteries and units may include location detection related components or systems, such as RTLS or RFID tags, to enable or enhance identification, tracking, and collection of battery health information (e.g., which may include use of a local handheld device) of multiple batteries at site (e.g., a hospital or warehouse).

Additionally, some embodiments enable, enhance or automate tracking of rules (e.g., government laws, statutes and regulations) that apply to batteries, units or medical devices, and determination of recommendations or actions to ensure compliance with potentially multiple applicable sets of such rules. For example, in some instances, multiple sets of rules (e.g., relating to different governmental entities or levels, and/or different regions) may apply to a battery, unit or medical device at a given location. In some embodiments, such rules may be collected, or automatically collected, kept updated, and analyzed to determine requirements and ranges for specific parameters (e.g., wireless transmission frequencies or data usage limits) that may apply to the battery, unit or other device at the location, which location may be communicated by transmissions from the battery, unit or other device itself. Some examples include determination of multiple set of rules that apply, and determination of parameter or usage settings or ranges that lead to compliance with all applicable rules, some of which may, for example, specify different but overlapping parameter or usage ranges for compliance, and determination of recommendations or actions accordingly.

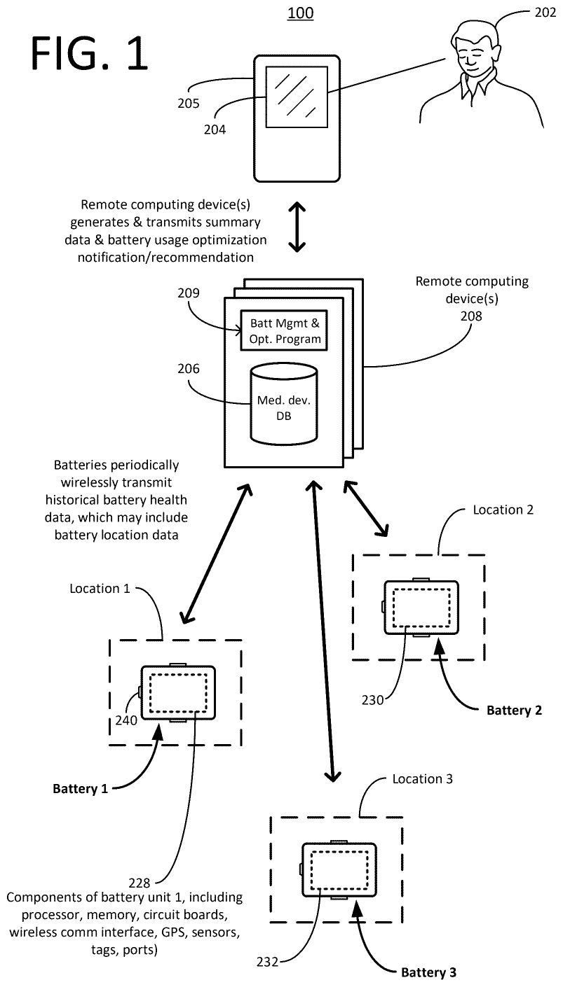

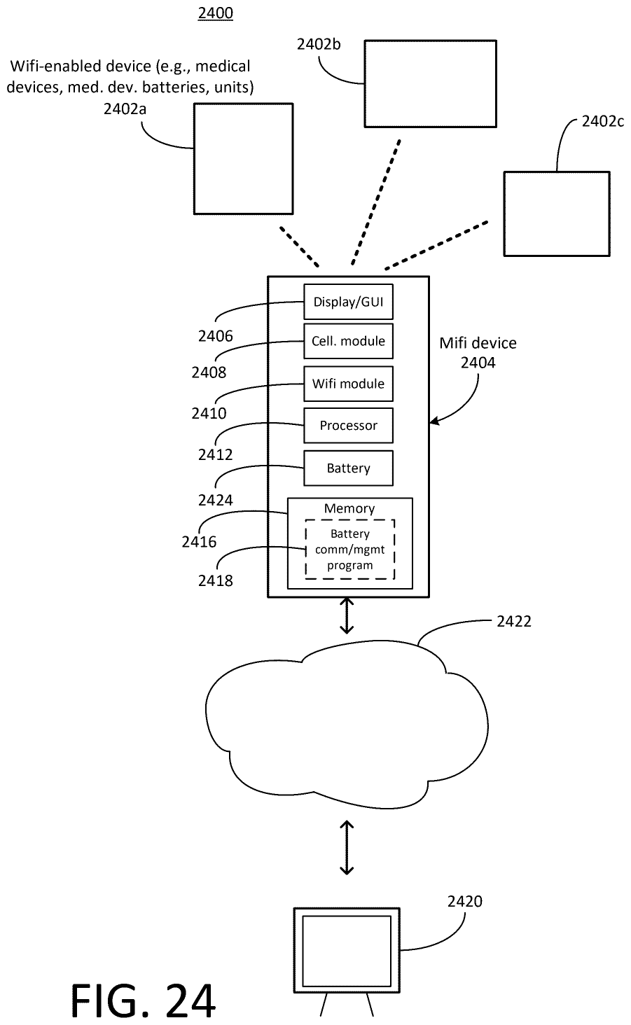

FIG. 1 is a block diagram 100 illustrating an example medical device battery management and optimization system 100, including a group of medical device batteries. Specifically, example medical device batteries 1, 2, 3 are shown. In the example, each of the batteries 1, 2, 3 are at different locations, specifically, locations 1, 2, 3, respectively. Each of the batteries includes components 228, 230, 232, including, e.g., a processor, a memory, a wireless communication interface (which may include, e.g., a transmitter and receiver, or a transceiver). In some embodiments, while the wireless communication interface may be powered by the battery 1, 2, 3, it may require a relatively small power output from a potentially high power output medical device battery. In some embodiments, wireless communication may be secured and require authentication. For example, in some embodiments, security and authentication could be used to require and ensure that communications and data sharing is only permitted with qualified or authenticated users and devices (e.g., including batteries or units, and components thereof), and that such communications are protected from interception, decoding or unintended access. In some embodiments, combinations of known software and techniques may be utilized in this regard, including use of login names and passwords, device IDs, encryption, hashing, custom certificates, private keys and/or others, which may be used, e.g., in ensuring that devices, such as batteries and units, reliably identify themselves and that communications with them are reliably secured. Additionally, in embodiments in which a mifi device is used (as described herein, e.g., with reference to FIG. 24), such security and authentication techniques may also be utilized, e.g., in relation to authenticating devices connected to a wifi device, and in relation to communications and data sharing enabled by the wifi device, for example. Furthermore, in the embodiment shown, each of the batteries 1, 2, 3 includes other components, including one or more processors, one or more memories, one or more circuit boards, a location manager (such as may include a GPS receiver and associated electronics and/or software), proximity detection/locator components (e.g., one or more passive or active locator systems or components), sensors (e.g., a motion sensor, such as an accelerometer, an battery temperature sensor, a battery environment temperature sensor, a battery humidity sensor, a battery environment humidity sensor), and one or more ports, such as for connected other computing or display devices. Each battery 1, 2, 3 may also include, for example, coupling features or mechanisms 240, such as for attachment with or in a connected device, such as a medical device (e.g., an AED) or dock. In some embodiments, the memory of each of the batteries 1, 2, 3 may also include stored programming for use with aspects of battery management and optimization as described herein. In some embodiments, a battery may be connected to and disconnected from a connected device.

In various embodiments, each medical device battery 1, 2, 3 may be of any of various types, such as non-rechargeable or rechargeable batteries, such as lithium-ion, Nickel-cadmium, Nickel-metal hydride, solid state batteries, or others, and have any of various components, types of components, and combinations of components, including both hardware and software. For example, each battery may include one or more electronic circuit boards, protection boards, printed circuit boards (PCBs) and/or integrated circuits (ICs), for use, e.g., in battery operation and monitoring and measuring battery parameters such as voltage and current, and, for example, for preventing over-charging, over-discharging, over-current, and short circuit protection. Furthermore, in some embodiments, a battery may include one or several cells (a battery with multiple cells may be called a battery or battery pack). In some embodiments, a battery may include a battery management system (BMS), which may include an electronic system (including, e.g., circuit boards and ICs) for monitoring and protection of the battery, or cells within the battery.

Each of the batteries 1, 2, 3 collects and stores battery health data over time (e.g., by the processor writing data to the memory), which may include chronologically ordered and/or time-stamped data. Periodically, each of the batteries 1, 2, 3 transmits historical battery health data, which includes past information relating to battery usage and may include battery location data. The historical battery health data may include chronologically ordered or orderable data (e.g., time stamped, time-ordered or otherwise chronologically indicated, marked or trackable data). In some embodiments, the historical battery health data may include, among other things, data associated with a connected device (e.g., a medical device) powered by the battery, which may include, e.g., data relating to operation of the connected device and clinical event data (e.g., use of the connected device with patients, such as use of an AED in patient monitoring and delivery defibrillation shocks or pacing). In various embodiments, such periodic transmissions may be set to occur at regular, or irregular, such as varying, time intervals, or may be based on one or more non-time based parameters, such as by being triggered by a threshold amount of data being stored, or a threshold amount of memory used, or in other ways.

The transmitted historical battery health data may be received by one or more remote computing devices 208 (which may include being received directly, or via one or more intermediary devices or systems), such as one or more central server computers, including one or more processors, and one or more memories that may include a software-based battery management and optimization program 209 and a medical device database 206. The battery management and optimization program 209 represents programming or software used in implementing various embodiments as described herein. The medical device database 206 may be used to store, among other things, the collected transmitted historical battery health data from each of the medical device batteries 1, 2, 3, as well as any other data used in various embodiments described herein.

An administrative user 202 may send a request, received by the remote computing device(s) 208 to access, for example, historical battery health data associated with a group of medical device batteries. For example, the administrative user 202 may send a request to obtain displayed data regarding a user-defined grouping of medical device batteries. The user-defined grouping may be, for example, the medical device batteries in a specified region or at a specified location (e.g., within a specific building), a particular user-specified set of medical device batteries, or any of various other types of groupings.

In response to the request, the remote computing device(s) 208 may generate and transmit, for display on a display 204 of a display device 205 (e.g., a computer, tablet, smartphone or inspection/tracking display device) of the administrative user 202, summary data relating to the group of medical device batteries. In various embodiments, the display device 205 and/or user 202 may be local or remote (e.g., at a remote central or management station, for example). For example, in various embodiments, the summary data may be displayed as a GUI with a list by medical device battery, and/or as a map showing the location of each medical device battery, as well as information about some or each of them. Additionally, the remote computing device(s) 208, using the historical battery health data stored in the medical device database 206, determines an optimization notification, which may include, e.g., a message, alert, or warning, for display, which includes a recommended action to optimize battery usage. Various examples of summary data, maps, and optimization notifications with recommended actions are provided in later figures. In some embodiments, the remote computing device(s) 208, using the battery management and optimization program 209, analyzes stored historical battery health data in determining the optimization notification, examples of which are provided in later figures. Furthermore, in some embodiments, a displayed GUI and/or map may allow the administrative user 202 to choose to implement the recommended action (as one example, implement a recommendation to change a wireless communication setting of one or more of the batteries), or to modify and then choose to implement the modified action, and the remote computing device(s) 208 may transmit one or more commands to the one or more batteries to implement the action. In various embodiments, notifications/recommendations may take a variety of different forms, including, for example, computing device displays, SMS or text messages, emails, voice mails, and others. In some embodiments, a user may receive displayed notifications/recommendations based on a priority scheme, such as high priority/red, medium priority/orange and low priority/yellow, for example.

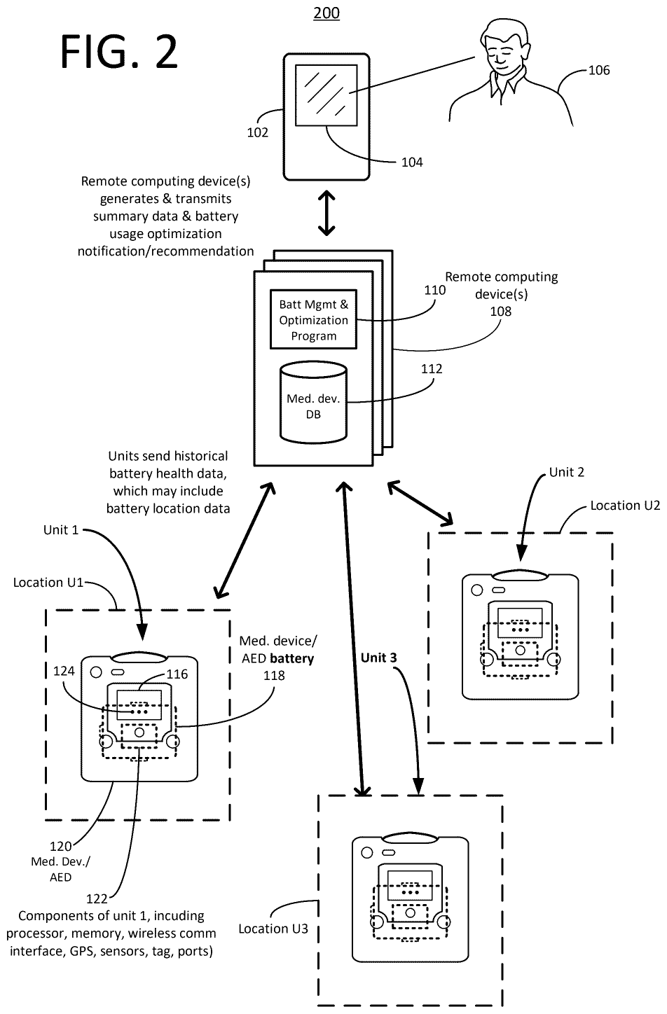

FIG. 2 is a block diagram 200 illustrating an example medical device battery management and optimization system, including a group of units 1, 2, 3, each including an AED and an AED (e.g., battery 118), physically and electrically connected with the AED. For each unit, each of the AED and the AED battery may include electrical contacts (e.g. electrical contacts 124 of unit 1) for electrical connection together. As described previously, in some embodiments, medical device batteries store and periodically transmit historical battery health data. However, in other embodiments, units do so, where each unit includes at least one medical device battery and a connected device, such as an AED or dock, and may include other components.

In some embodiments, for each unit, using a processor of the connected device, the connected device may obtain and store, in a memory of the connected device, historical battery health data relating to the one or more batteries connected to the connected device. However, in other embodiments, for each unit, the battery may obtain and store the battery health data, even when connected to the connected device, and the connected device may obtain and store the battery health data from the battery after the battery first obtains and stores it. Alternatively, in some embodiments, both the connected device and the battery may have roles in obtaining, storing and/or transmitting battery health data, and/or receiving communications or commands from a computing device(s), and may operate in an integrated fashion. Furthermore, in various embodiments, a battery may be connected to and disconnected from the connected device, such as may include being removed from and placed with or into an AED or dock, for example. In some embodiments, when the battery is disconnected from the connected device, the battery may obtain, store and periodically transmit battery health data, which may include historical battery health data, independently from the connected device, regardless of whether the battery does so when connected to the connected device.

In the embodiment shown, example units 1, 2, 3 are shown, at different respective locations U1, U2, U3, each including an AED (such as AED 120 of unit 1), AED battery (such as AED battery 118 of unit 1), each of which may include a display (e.g., display 116 of unit 1), and each unit may include various other components (e.g., components 122 of unit 1), such as one or more processors, one or more memories, and a wireless communication interface, and may include other components including a location manager (such as may include a GPS receiver and associated electronics and/or software), proximity detection components (e.g., passive or active locator systems or components), sensors (e.g., a motion sensor, such as an accelerometer, a battery temperature sensor, a battery environment temperature sensor, a battery humidity sensor, a battery environment humidity sensor), and one or more ports, such as for connected other computing or display devices. In various embodiments, these various components may be distributed in various different ways and combinations between the AED and the AED battery. For example, in some embodiments, for each unit 1, 2, 3, the AED includes a processor, a memory, a wireless communication interface, a location manager, proximity detection components, and ports, and the AED battery includes an accelerometer, a battery temperature sensor, a battery environment temperature sensor, a battery humidity sensor, a battery environment humidity sensor. However, many other arrangements and combinations may be used in other embodiments.

As depicted in FIG. 2, each of the units 1, 2, 3 periodically transmits historical battery health data, which may include battery location data (relating to the one or more batteries of the unit), and data associated with the AED, which may include, e.g., data relating to operation of the AED and/or clinical event data (e.g., use of the connected device with or in associated with patients, such as use of an AED in patient monitoring and delivery of defibrillation shocks or pacing).

The remote computing device(s) 108, which includes a battery management and optimization program 110, receives and stores the historical battery health data in a medical device database 112. In some embodiments, upon receipt of an administrative user request, such as regarding a user-defined grouping of units, the remote computing device(s) 108 may generate and transmit, for display on a display 104 of a display device 102 of the administrative user 106, summary data relating to the group of units. The remote computing device(s) 108, using the historical battery health data stored in the medical device database 112, determines an optimization notification (e.g., a message, alert and/or warning) for display, which provides a recommended an action to optimize battery usage, with regard to one or more medical device batteries of one or more of the group of units.

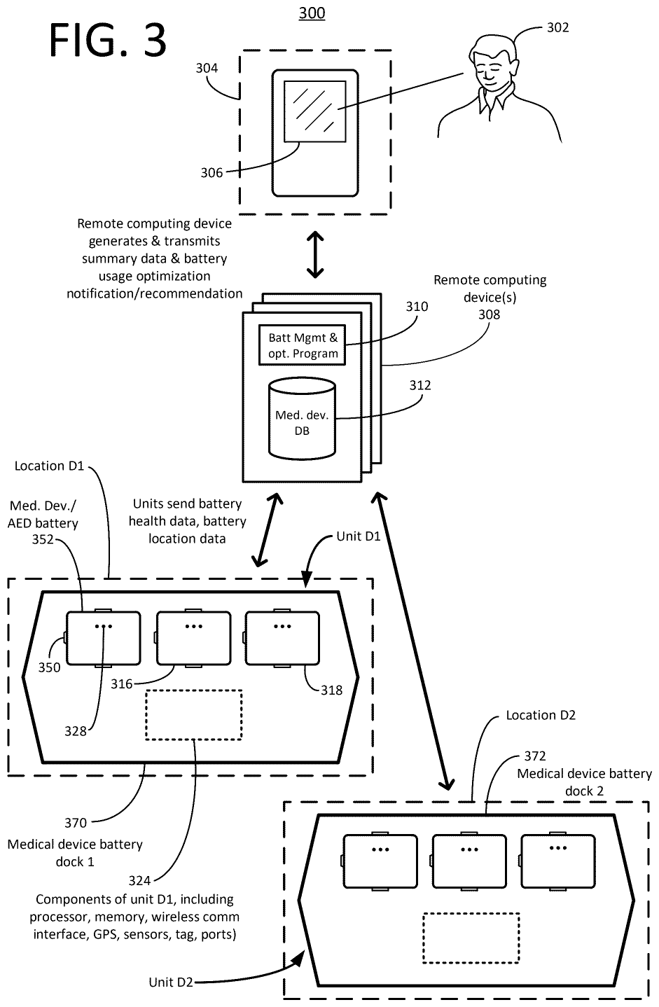

FIG. 3 is a block diagram 300 illustrating an example medical device battery management and optimization system, including a group of units (e.g., D1, D2) at locations D1, D2 respectively, each including a medical device battery dock 370, 372 and medical device batteries (e.g., AED battery 352 of unit D1). As shown, each unit D1, D2 includes three medical device batteries (e.g., batteries 350, 316 and 318 of unit D1). For each unit D1, D2, the dock and each battery may include electrical contacts (e.g., electrical contacts 328 of unit D1) for electrical connection together (when the batteries are in docked in the dock).

In some embodiments, medical device battery docks are provided, where each dock may be able to include or house one or multiple medical device batteries (e.g., 2, 3, 4, 5, 6-10, 11-20 or 21-50 batteries). In some embodiments, as described with reference to FIG. 2, a dock may obtain and store battery health data from each docked battery, or from some of the docked batteries, and various embodiments are possible regarding the roles of the dock and the batteries with regard to, e.g., obtaining, storing and transmitting battery health data, whether the battery is connected or disconnected from the dock (e.g., in some embodiments, even when docked, batteries may obtain, store and/or transmit battery health data, or have a role therein). In various embodiments, the dock itself may be powered by wired connection to a power outlet, or by its own battery, or either, and, where applicable, the dock battery may be charged in various ways and by various types of chargers. In some embodiments, docks may be especially suited for military use, where the dock may protect the batteries from motion during transport, shock, harsh environmental conditions such as high or low temperature or high humidity, or other conditions.

In some embodiments, in addition to having a role in obtaining, storing and transmitting battery health data, the dock may serve one or more roles. In some embodiments, the dock may include a charger or charging coil(s), and may serve as a wired or wireless charger for the connected batteries. For example, in some embodiments, when inserted and secured into the dock, each battery is charged by wired connection to the dock. In other embodiments, the dock may include one or more coils for wireless charging of the batteries when the batteries are inserted into the dock, or may be equipped for either wired or wireless charging of batteries. Additionally, in various embodiments, the dock may provide varying degrees of physical protection (e.g., from substances, damage or physical shock) and electrical protection for the connected batteries. For example, in some embodiments, each battery is inserted partially or completely into the dock so that the dock partially or completely physically covers or houses them, or the dock may open and close so that batteries may be inserted, and then the dock closed to partially or fully secure, house and physically protect the batteries. Additionally, in some embodiments, the dock provides electrical insulation and protection to the batteries, shielding them from potential external electrical disturbances. As such, in various embodiments, docks may serve as containers for batteries, and may provide, for example, charging for batteries as well as communications capabilities relating to batteries and battery health data.

Each of the example units D1, D2 includes a dock (dock 1, dock 2), multiple medical device batteries (e.g., in unit D1, batteries 350, 316, 318), and various other components (e.g., components 324 of unit D1) such as one or more processors, one or more memories, and a wireless communication interface, and may include other components including a location manager (such as may include a GPS receiver and associated electronics and/or software), proximity detection components (e.g., a real-time location system (RTLS) tag or radio frequency identification (RFID) tag), sensors (e.g., a motion sensor, such as an accelerometer, a battery temperature sensor (e.g., for each battery), a battery environment temperature sensor (e.g., for all batteries or for each battery), a battery humidity sensor (e.g., for each battery), a battery environment humidity sensor (e.g., for all batteries or for each battery), and one or more ports, such as for connected other computing or display devices. In various embodiments, these various components may be distributed in various different ways and combinations, between the dock and the connected batteries. For example, in some embodiments, each connected/docked battery may include one or more of an accelerometer, a battery temperature sensor, a battery environment temperature sensor, a battery humidity sensor, a battery environment humidity sensor. However, many other arrangements and combinations may be used in other embodiments.

As depicted in FIG. 3, each of the units D1, D2 periodically transmits historical battery health data, which may include battery location data (relating to the one or more batteries of the unit), and may include other data, such as data relating to a medical device to which each battery may have been connected and powered before being connected/docked, for example.

The remote computing device(s) 308, which includes a battery management and optimization program 310, receives and stores the historical battery health data in a medical device database 312. In some embodiments, upon receipt of an administrative user request, such as regarding a user-defined group of units, the remote computing device(s) 308 may generate and transmit, for display on a display 306 of a display device 304 of the administrative user 302 (or elsewhere, such as locally, on a display of an AED, or remotely), summary data relating to the group of units. The remote computing device(s) 308, using the historical battery health data stored in the medical device database 312, determines an optimization notification (e.g., a message, alert and/or warning) for display, which recommends an action to optimize battery usage, with regard to one or more medical device batteries of one or more of the group of units.

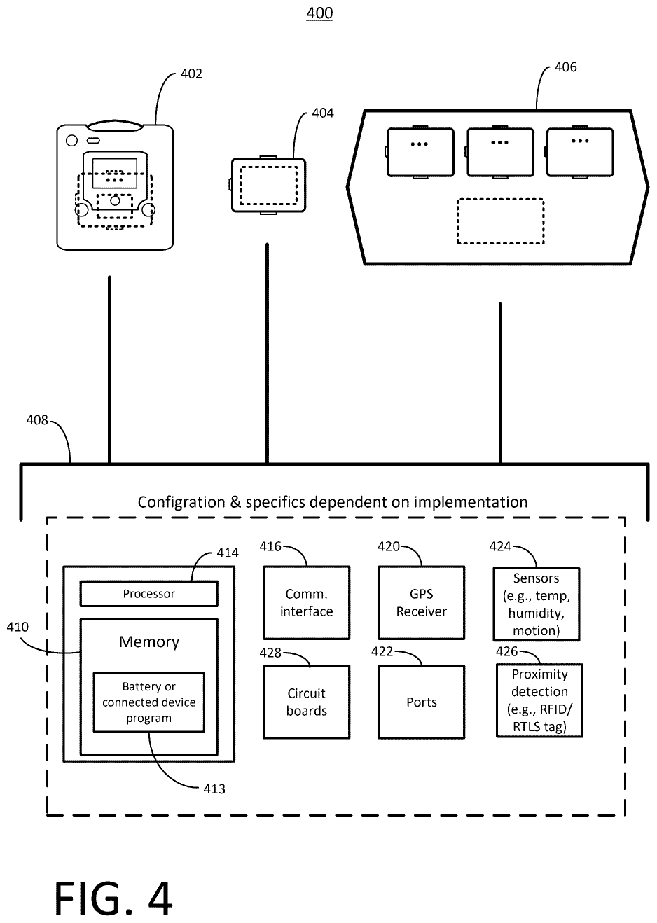

FIG. 4 is a block diagram 400 illustrating components 408 of example medical device battery management and optimization systems. As described previously, in various embodiments, various components of medical device batteries and connected devices, such as AEDs or docks, may be distributed in various ways between the batteries and connected devices. In some embodiments, however, each battery (if no connected device) or each unit may include some or all of the depicted components. A processor 414 and memory 410 may be included. The memory 410 may include a software-based battery or connected device program 413, which represents programming or software used in implementing various embodiments described herein, such as by the processor 414, which, in some embodiments, may include, e.g., obtaining, storing and transmitting battery health data, or having a role therein. In some embodiments, the processor 414 may also be used in various other tasks, such as, for example, receiving communications from a remote computing device, such as to implement actions, such as changing or adjusting a battery related setting, for example. It is noted that, in various embodiments, each of the processor 414, the memory 410, and the battery or connected device program 413 may be implemented in a distributed fashion, on multiple devices. This could include, for example, multiple distributed processors, multiple distributed memories, and multiple distributed portions of a battery or connected device program, which work in an integrated or unified fashion.

Additionally, other components may be included, such as in medical device batteries 404 and connected devices, including, e.g., AEDs 402 or docks 404, as described herein in various embodiments. These may include a communication interface 416 (e.g., a wireless and/or wired communication interface), a GPS receiver 420 (or other location sensing or determining component), various sensors 424 (e.g., battery temperature sensors, battery environment temperature sensors, battery humidity sensors, battery environment humidity sensors, motion sensors such as accelerometers), ports 422 (such as USB ports or others), for connection of various devices, such as computing devices, display devices, or batteries or other power related devices, proximity detection components 426 (such as RTLS tags or RFID tags), and various other electrical components including circuit boards 428.

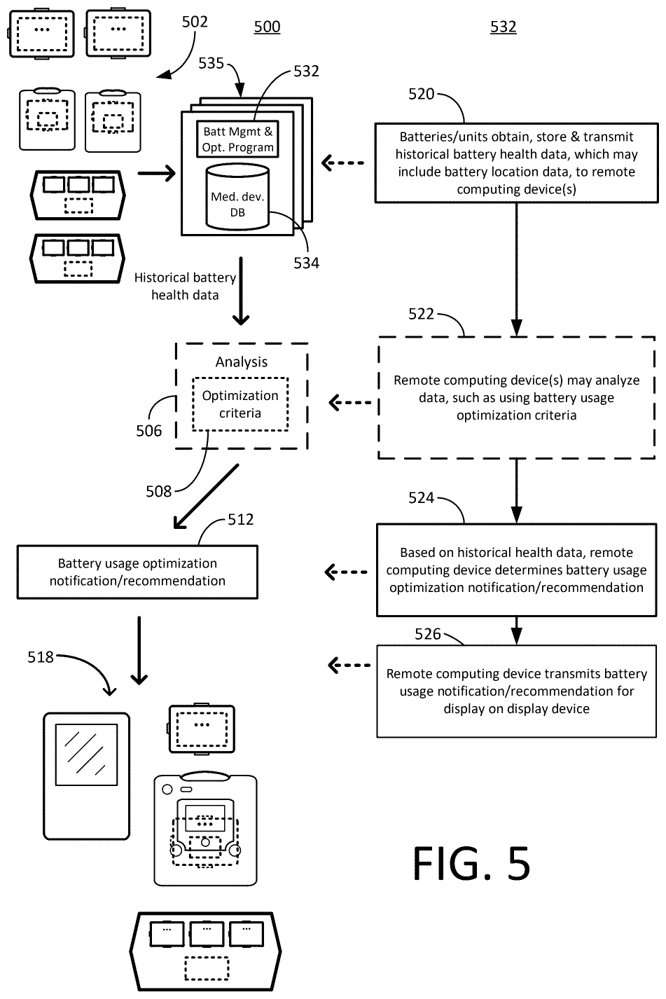

FIG. 5 includes a flow diagram 500 according to an example medical device battery management and optimization method. At step 520, batteries (if no connected device is included) or units (if a connected device, such as an AED or dock, is included) obtain, store, and transmit data, including historical battery health data, to a remote computing device(s) 535. The remote computing device(s) 535 may include use of a battery management and optimization program 532 and medical device database 534. In some embodiments, the historical battery health data includes battery location data, although, in some embodiments, battery location data is not included in the historical battery health data or in the obtained, stored and transmitted data. Blocks 502 represents various possibilities with regard to obtaining, storing and transmitting devices, which can include, e.g., batteries, medical devices such as AEDs, or docks. Furthermore, in some embodiments, combinations including batteries, AEDs and/or docks (or other types of connected devices) may be included among the obtaining, storing and transmitting devices.

At step 522, in some embodiments, the remote computing device(s) may perform analysis 506, such as analysis of data including historical battery health data, to determine optimization notifications and recommended actions to optimize battery usage, which may, in some embodiments, include use of optimization criteria 508, as further described herein.

At step 524, based on data including the historical battery health data, the remote computing device(s) 535 determines a battery usage optimization notification/recommendation 512. At step 526, the remote computing device(s) 535 transmits the optimization notification/recommendation 512, including a recommended action to optimize battery usage, for display on a display device 518, such as a display device of an administrative user. In some embodiments, the optimization notification/recommendation may be displayed to any of various other types of users, which could include remote user, local users, and could include remote or local care providers. Furthermore, in some embodiments, the display device could be or include a computing device, such as a computer, tablet, or smartphone, or a medical device with a display, such as a defibrillator such as an AED or other device, or a display device of remote computing platform, for example. To provide an example, the foregoing may include analyzing battery temperatures or charge levels, and determining recommendations relating to, e.g., reducing battery charging temperatures or avoiding storing the battery at full charge or zero change (either of which may reduce battery capacity). In other examples, the foregoing may include analyzing wireless signal/reception levels at battery locations and during times of wireless connections or connection attempts, and making recommendations accordingly, such as to move one or several batteries to a location with better signal/reception, to reduce connection frequency, or to attempt to connect during times when signal/reception is likely to be stronger, based on historical information. These and other examples are described in detail in later figures.

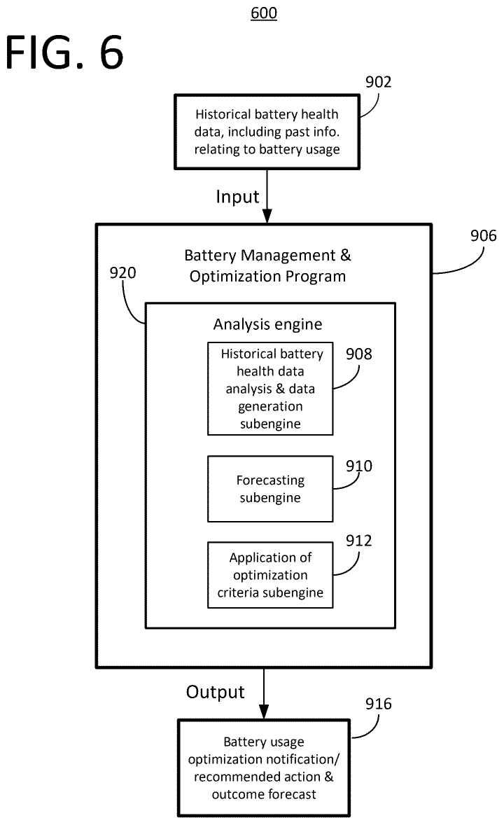

FIG. 6 is a block diagram 600 illustrating example use of a battery management and optimization analysis engine 920. Block 902 represents historical battery health data, including past information relating to battery usage, such as may be collected by or from medical device batteries, which is used as input to a battery management and optimization program 906, such as of a remote computing device(s).

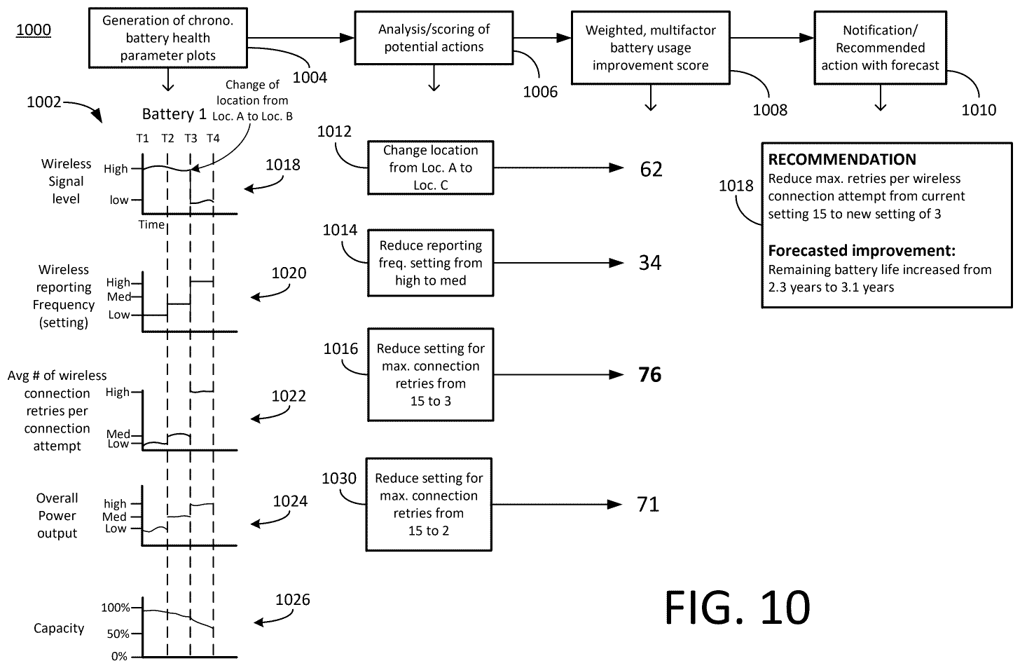

As depicted, the analysis engine 920 includes a historical battery health data analysis and data generation subengine 908, a forecasting subengine 910 and an application of optimization criteria subengine 912. In some embodiments, the historical data analysis and data generation subengine 908, which may be used in analyzing data including historical battery health data, such as chronologically ordered or orderable data or time-stamped data relating to specific battery parameters, in generating data sets or data structures (e.g., plots, which may be generated for internal use and/or display). The data sets or data structures, in turn, can be used in determination of optimization notifications, including recommended actions, and, in some embodiments, can be used in forecasting, such as may be used in determining recommended actions as well as projected results from specific recommended actions or potential recommended actions. The data sets or data structures may, for example, organize chronological historical battery health data in a form and structure that permits analysis to determine optimization notifications and forecasts. This may include data sets or data structures that include organized, time-tracked data for a single battery parameter (e.g., battery temperature during charging or setting for a wireless reporting frequency). Additionally or alternatively, it may include organized, time-tracked data relating to two or more such parameters. Furthermore, it may include data sets or data structures generated in complex ways from historical battery health data, such as data sets or data structures that are determined using chronologically ordered data relating to one or more battery related parameters, but are generated using some manipulating or transforming function, operation or algorithm (e.g., use of a regression algorithm to, model a battery related parameter that is not explicitly characterized by the historical battery health data, as described further below). The data sets or data structures may also relate to calculated trends and patterns, for example. A detailed example of operation of an analysis engine is provided with reference to FIG. 10, in which a set of time-based battery related parameter plots is generated and used in analysis including forecasting.

The analysis engine 920 may use forecasting, such as of potential outcomes or results of potentially or actually recommended actions. In some embodiments, a forecasting subengine 910 is used in determining forecasts. A detailed example of forecasting is provided with reference to FIG. 10, in which forecasts associated with a set of specific potential recommended actions is used in determining a recommended action, and potentially in displaying a forecast of a projected result or outcome of implementation of the recommended action. Forecasting may be used in assessing the magnitude of improvement projected to result from particular recommended actions or potential recommended actions. Forecasting may also be used, for example, to determine a particular optimal adjustment amount from a range of potential such amounts, for example.

Block 912 represents application of optimization criteria, for example, to determine potential recommended actions, or to determine which, of a group of potential recommended actions, to select as a recommended action. For example, application of optimization criteria may include use of forecasts to assess specific recommended actions, an example of which is provided with reference to FIG. 10, such as to determine which is optimal.

Block 916 represents output of the analysis by the analysis engine, including a battery usage optimization notification including a recommended action and, in some embodiments, an outcome forecast.

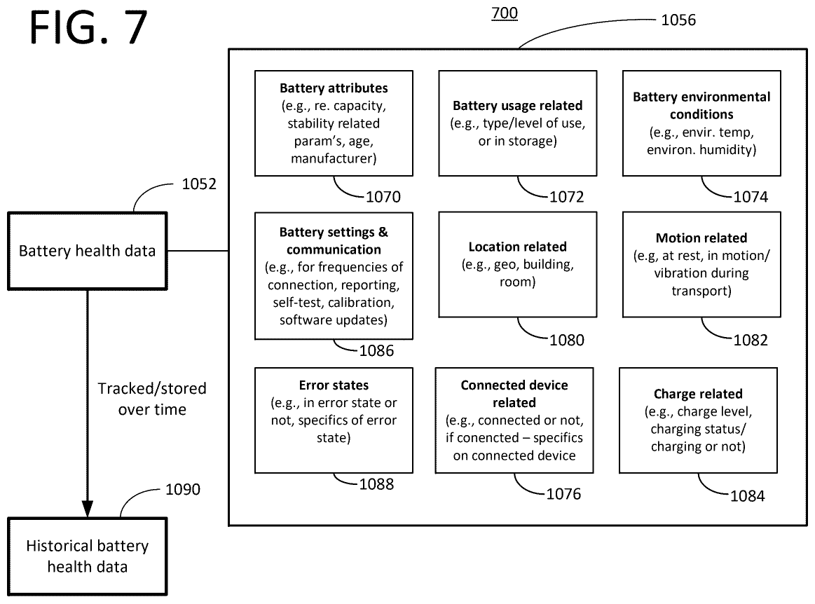

FIG. 7 is a block diagram 700 illustrating example types 1056 of medical device battery health data 1052, which may be used in various embodiments described herein. The types 1056 are not intended to be comprehensive or mutually exclusive, and some data may be of multiple types. Battery health data can include, e.g., any of various battery parameter data. Furthermore, battery health data can include, e.g., historical battery health data. Historical battery health data can include, e.g., past information relating to battery usage. In various embodiments, the data may be stored in different ways, forms, and formats, and using different types of data structures, such as, e.g., in tables of a relational database, hash tables, linear data structures, tree data structures, arrays, queues, linked lists, stacks, heaps, graphical data structures, data files, comma-separated value formats, or others.

Some examples of battery health data 1056 may include, e.g., battery condition, battery status, battery usage, battery error states, battery type, battery manufacturer, battery manufacture date, battery serial number, battery specification information, battery age, battery location, connected device, clinical event data associated with the connected device, battery temperature, battery environment temperature, battery humidity, battery environment humidity, battery motion, battery capacity, battery design capacity, battery remaining capacity, battery power output, battery remaining operating time, battery alarms, battery remaining capacity alarm, battery remaining operating time alarm, battery mode, battery stability, battery reliability, cycle life and battery calibration. Some examples of battery health data that may, e.g., be associated with communication may include battery wireless communication, battery periodic reporting, and battery periodic software updating. Some examples of battery health data that may, e.g., be associated with communication may include battery wireless communication, battery periodic reporting, battery periodic software updating. Some examples of battery heath data that may, e.g., associated with charging may include battery charging, battery charging rate, battery absolute state of charge, battery relative state of charge, depth of discharge, battery full depth of discharges. Some examples of battery health data that may, e.g., associated with various electrical parameters may include minimum lifetime battery pack voltage, maximum lifetime battery pack voltage, minimum lifetime battery pack temperature during discharge, maximum lifetime battery pack temperature during discharge, maximum lifetime battery pack temperature during charge, maximum lifetime current during charge, maximum lifetime current during discharge, total discharge amp-hours throughput, total charge amp-hours throughput, energy density, specific energy, nominal capacity, nominal energy, terminal voltage, open-circuit voltage, cut-off voltage, nominal voltage, charge voltage, float voltage, discharge current, C-rate, recommended charge current, specific energy, energy density, specific power, and power density. Various categories of battery health data are described below, which categories may include various examples of battery health data listed above, among others.

Battery health data 1056 can include historical battery health data 1090, which may include battery health data tracked over time. In some embodiments, historical battery health data may include time-stamped data for many times over a period of time. For example, a time stamp may include the date and time of day that certain battery status parameters are obtained—e.g., date, time, battery voltage (e.g., 11180 mV), battery state of charge (e.g., 68%), battery average current (e.g., 1255 mA), etc. Historical battery health data 1090 may include, e.g., data tracking any of the types of battery health data over a period of time.

One type of medical device battery health data 1056 includes data relating to various battery attributes 1070, which may include, for example, various types of battery parameters, such as parameters relating to capacity, stability, voltage, current, impedance, other electrical parameters, and battery conditions such as battery temperature, battery humidity, and others. For example, such data could include average battery current. Furthermore, in some embodiments, battery attributes may relate to each, several or all of multiple cells of a battery (e.g., the voltage of each cell).

Other battery parameters may relate to various background or status information relating to a battery, such as, for example, age, parameters relating to battery manufacture (e.g., manufacturer, manufacture date), serial number, various battery specifications, and others. Additionally, other battery parameters may relate to aspects, features and details of various hardware, software and settings, that are part of, or used by, or to be used by, the battery, such as, for example, specific physical components, mechanical components, processor, memory, circuit boards, electronics, various programming and software, versions of software, and others.

Some battery parameters relate to capacity. Capacity represents the total amount of electrical energy that can be generated by the battery, and may be measured in units such as ampere-hours (Ah) or watt-hours (Wh). Absolute capacity may refer to the capacity of the battery when it is new. Relative capacity may refer to the current capacity of a battery relative to the absolute capacity of the battery, and is sometimes expressed as a percentage. Capacity may refer simply to the current capacity of a battery, such as may be measured in Ah or Wh. A certain minimum battery capacity may be required when needed to power a medical device for a particular use and period. For example, for a defibrillator, such as an AED, a large power output may be required to delivery one or several high power defibrillation shocks, requiring a significant amount of battery capacity. As such, battery capacity can be critical to a medical device being able to deliver potentially life-saving treatment.

Battery capacity decreases over time, although at various rates, so ensuring that medical device batteries have sufficient capacity, or will have sufficient capacity over a period of time of anticipated use or potential use, can be critical. However, the rate at which capacity decreases over time may depend on many factors, some of which include charging related parameters such as charging patterns, battery temperature during charging, and battery humidity during charging. For example, the total amount of charging during cycles (whether full or partial) experienced by the battery may reduce battery capacity, so that greater battery use (and associated charging) generally correlates with more or more rapid capacity decline. As another example, maintaining a lithium-ion battery at a charge level of nearly a 100% of the battery capacity for long periods of time (e.g., via trickle charging), or storing a battery at 0% capacity for a long enough time, can reduce capacity or even end the life of a battery. As still another example, high battery temperature during charging can reduce capacity. Capacity may be characterized using difference units (e.g., ampere-hours (Ah) or watt-hours (Wh)), may be specified in different ways (e.g., relative capacity may express the battery's capacity as a percentage of when it was new (which may represent the battery's absolute capacity) and may be specified based on specific or standard conditions of measurement (e.g., nominal energy, which relates to Wh available under specified conditions of temperature and load).

Some battery parameters may relate to battery stability. Stability can be defined, as well as measured or estimated, in various ways, and, in some embodiments, may include user-configurable aspects. For example, a stability related parameter may be defined to characterize a battery's risk of failure (e.g., failing to produce an expected use or power output, or failing altogether in the sense of being no longer usable to produce any power output), or reliability (e.g., probability or confidence level relating to maintaining parameters or usage capability within required, specified, or user-specified or configured parameters or ranges). In some embodiments, for example, battery stability may be defined, characterized or measured using information including current and/or historical battery health data. For example, in some embodiments, battery health data may be analyzed to determine how closely, or how statistically closely (e.g., based on a calculated degree of deviation) recent and/or current battery parameters (e.g., electrical parameters such as voltage and capacity), and accuracy of battery monitoring of its parameters (e.g., electrical parameters) and/or usage (e.g., operating temperature,) match to an ideal or high performance range. Furthermore, in some examples, battery stability may take into account how closely battery parameters or usage track predicted ranges of such parameters or usage over time (e.g., the rate at which battery capacity declines with usage and under various conditions, such as temperature ranges), as well as the range of various conditions at which battery parameters or usage remains within sufficient or ideal ranges (e.g., conditions such as ranges of operating, charging temperatures, or shock/motion (which may include acceleration) related conditions.

Additionally, in some embodiments, batteries or units are monitored, and/or monitor themselves, with regard to parameters and usage, over various ranges of conditions (e.g., temperature, humidity and shock/motion conditions, which may include conditions within and outside of requirements or limits). The results may be used in determining or measuring (which may include estimating) a battery's stability.

Another example stability related parameter may be defined to characterize a battery's risk, or risk over time, of experiencing some particular problem, or one of a set of problems, or an error state or one or a set of error states (error states are described further herein). Such problems or error states could, for example, reduce a battery's ability to optimally perform or produce an expected outcome, or could involve dangerous conditions. Various specific types of battery stability can include chemical stability (e.g., avoiding unintended chemical reactions and achieving desired ones), electrochemical stability (e.g., relating to the interface between the battery electrolyte and either the battery cathode or the battery anode), mechanical stability (e.g., relating to a battery's ability to withstand motion, shock or physical damage without compromising performance), and thermal stability (e.g., avoiding unwanted battery changes that may occur with battery heating, or, in some cases even catching fire). Factors that may decrease battery stability may include, e.g., age, battery motion or shock, battery over-heating or over-humidity, or physical battery damage, for example. In some examples, measures of battery impedance may be used in estimating battery stability, since increased battery impedance may be associated with decreased battery stability. In some embodiments, various types or definitions of stability may be used, and stability may be measured or estimated in various ways (e.g., based on historical battery health data relating to factors such as age, impedance, battery motion or shock, physical battery damage, or others).

Some battery parameters may relate to battery voltage, which relates to the amount of electrical potential that a battery can hold, and may be measured in volts (V). Such parameters may include, for example, battery voltage, depth of discharge (relating to a percentage of battery capacity that has been discharged), terminal voltage (relating to a voltage between battery terminals with a load applied), open-circuit voltage (relating to a voltage between battery terminals with no load applied), cut-off voltage (relating to a minimum allowable voltage), and nominal voltage (relating to a reported, reference or normal voltage), charge voltage (relating to the voltage that the battery may be charged to), and float voltage (relating to a voltage at which the battery can be maintained under certain conditions).

Battery usage-related parameters 1072, such as may relate to type of use (e.g., which may relate to operation of the medical device powered by or to be powered by the battery), level or magnitude of use (e.g., total power output in Ah or Wh, total operating time, and others). Some battery parameters relate to battery mode. In some embodiments, for example a battery may have several modes, including a sleep or standby mode, e.g., lower power or current output, and an active mode, e.g., with higher power or current output, or may have more than two modes. For example, a battery may periodically or at specific times or intervals wake up from a sleep or standby mode in order to wirelessly transmit and/or receive data, such as to periodically transmit historical battery health data. Additionally, the battery may wake up if an external device initiates communication with the battery, whether locally (e.g., by local wireless communication, which may include passive or active locator systems, or by wired connection via a port on the battery or unit) or remotely by wireless communication. Furthermore, in some embodiments, battery transmission and/or receipt of data may require secure and authenticated communication, and the battery or unit may include hardware and/or software related thereto.

Battery environmental conditions 1074 may include, e.g., battery environment temperature and battery environment humidity. It is noted that these parameters relate to conditions external to the battery, and are distinct from battery temperature and battery humidity, which relate to conditions internal to the battery. For example, battery temperature may be affected by environmental temperature, but may also be affected by other factors, such as charging or use, which may increase battery temperature above the environmental temperature. Furthermore, batteries may be protected from environmental humidity, or even surrounding liquids, and, as such, battery humidity may differ from environmental humidity. In various embodiments, batteries may include internal sensors for sensing battery temperature and battery humidity, and batteries or connected devices may have sensors (e.g., attached to a housing of the battery or connected device) for sensing battery environment temperature and battery environment humidity.

Battery connected device related parameters 1076 may include any of a variety of parameters relating to a connected device that is or may be connected to a battery (e.g., a medical device such as an AED, or a dock), particularly as may be associated with powering provided by the battery (e.g., parameters relating to medical device usage powered by or to be powered by the battery). For example, the electrodes or electrode pad (e.g., adult or pediatric) used by an AED may affect the power necessary to provide defibrillation shocks using the AED, which may in tern affect battery power output required or anticipated to be required to power the defibrillation shocks.

Various battery location related parameters 1080 may be used in various embodiments. This may include, for example, parameters relating to a geographic location of a battery (e.g., state, country, city, block) or more local/granular parameters, such as a specific building where the battery is located, or a specific part of the building, floor, room, portion of a room, etc. Additionally, various other parameters may be used that are associated with the location itself, such as ambient temperature, ambient humidity, wireless signal levels.

Battery parameters may include motion-related parameters 1082, such as, e.g., lack of motion or motion associated with, or determined to be associated with, storage (non-movement), transport (e.g., vibration) and shock (e.g., from being dropped, handled roughly or subjected to accelerations, which may be measured, e.g., using one or more accelerometers, for example). Additionally, battery parameters may include whether a battery is connected to or in another device, such as a connected device (e.g., AED or dock) or charger. In some embodiments, patterns of parameters associated with specific locations may be determined or generated and used, such as, for example, an average amount or type of battery use at the location, or movement or shock (e.g., from being dropped) experienced by batteries at that location over a past period of time, or forecasted to occur during a future period of time. For example, in some examples, exposure or a battery to excessive shock or motion related conditions may reduce battery stability, such as by, e.g., fracturing a PCB of a controller, loosening or separating internal connections or wiring, compromising or reducing soldering conditions or quality, or in other ways, which could, e.g., increase the risk of battery failure or not meeting battery parameter or usage requirements or expectations.

Various charge or charging related parameters 1084 may also be used in various embodiments. These may include, for example, parameters relating to charging patterns (e.g., frequency of charging, number of charging cycles, charge current, battery and environment temperature and humidity during charging, and various parameters mentioned above that are associated directly or indirectly, with charging or discharging (e.g., state of charge, discharge current, depth of discharge). For example, charging related battery health data may indicate, at a specific time, how much time, or how much more time, is forecasted to be required for full charge, or how much more runtime is forecasted to be provided at a current power output level before the battery is fully discharged, or alarms relating to any of the foregoing.

Battery parameters relating to various battery related settings 1086 and wireless communication parameters may also be used in various embodiments. These may include, for example, wireless communication related settings, such as wireless connection frequency, conditions required for wireless connection attempts (e.g., signal level), number of retries per connection attempt, wireless reporting frequency (e.g., where the wireless reporting could be initiated or transmitted from a battery or connected device) or conditions, wireless software update frequency or conditions, and wireless connection software protocol (e.g., handshake software protocol), or software version/version history. Battery related setting may also include, for example, calibration conditions and frequency (whether done in-person or automatically), and others.

Battery parameters may also relate to battery errors or error states 1088, and may indicate, for example, whether a battery is (or was) in an error state (or not), the specific error state, and specifics or conditions about the error state, for example. Many different types and levels of generality of error states, and associated error state categories or messages, warnings, alarms, etc., may be used in various embodiments. These may relate, for example, to physical, mechanical, chemical, electrical, or computerized structure, operation, or configuration of the battery, among other things. For example, error states may include electronics or electrical parameter related faults, such as a battery voltage fault, a battery pack voltage fault, an under voltage fault, an over voltage fault, a capacity fault, an absolute capacity below limit fault, an absolute capacity below limit fault, a current fault, a PCB fault, an initial PCB power-on fault, a loss of power to PCB fault, an electronic circuit fault. They may further include software or computer related faults, such as a fault of the processor, a fault of the memory, a software fault, a computer chip fault, a power-off fault, a reset fault, a clock frequency fault, a watchdog timer reset fault, an alarm fault, a cyclic redundancy check fault, a flash cyclic redundancy check fault, an integrated circuit power supply pins fault, a battery fuel gauge fault, a fuel gauge communication fault, a fuel gauge safety error fault, a VCC supply brownout, a CPU fault, a blown fuse fault, a sensor fault, a call stack overflow fault, a call stack underflow fault, an invalid configuration fault, a software initiated reset fault, a serial flash error fault, an invalid cyclic redundancy check on flash data fault, a microcontroller RAM fault, a battery pack disabled fault, a software interrupt fault. They may also include various other types of faults and specific faults, including, for example, a battery temperature fault, a battery under temperature fault, a battery over temperature fault, an initial reconditioning charge cycle required fault, a charge protocol fault, a charge too long fault, a stuck button fault, and charge protocol fault.

Furthermore, some battery parameters may relate to warnings or alarms. For example, an alarm may be triggered when remaining battery capacity has dropped below a threshold, e.g., for an AED battery, if a relative capacity of at least a specific percentage (e.g., 60% of absolute capacity) required for usage to power an AED, or when the remaining battery life time drops below a specific limit (e.g., a certain number of days, week, or months). Historical battery health data may include historical data tracking a history of such triggered alarms, for example. Historical battery health data may also include, for example, counters relating to a number of times that specific battery related event, condition, or error state has occurred, for example.

Many other battery parameters may be used in various embodiments. These include, for example, discharge current (sometimes measured as a C-rate, which may represent a measure of the rate at which a battery is discharged relative to its capacity, and may be measured in watts (W)), maximum continuous discharge current (relating to the maximum current at which the battery can be discharged continuously), recommended charge current (relating to the ideal current at which the battery is initially charged), cycle life (relating to the number of charge cycles that the battery can experience before failure to meet specified performance criteria), specific energy (relating to the nominal battery energy per unit mass), specific power (relating to the maximum power available per unit mass), energy density (relating to the nominal battery energy per unit volume), and power density (relating to the maximum allowable power per unit volume). Battery parameters could also include information about battery servicing or calibration (e.g. a log of servicing or calibration dates, or last serviced or calibrated dates, amount of time or power output since last servicing or calibration, etc.).

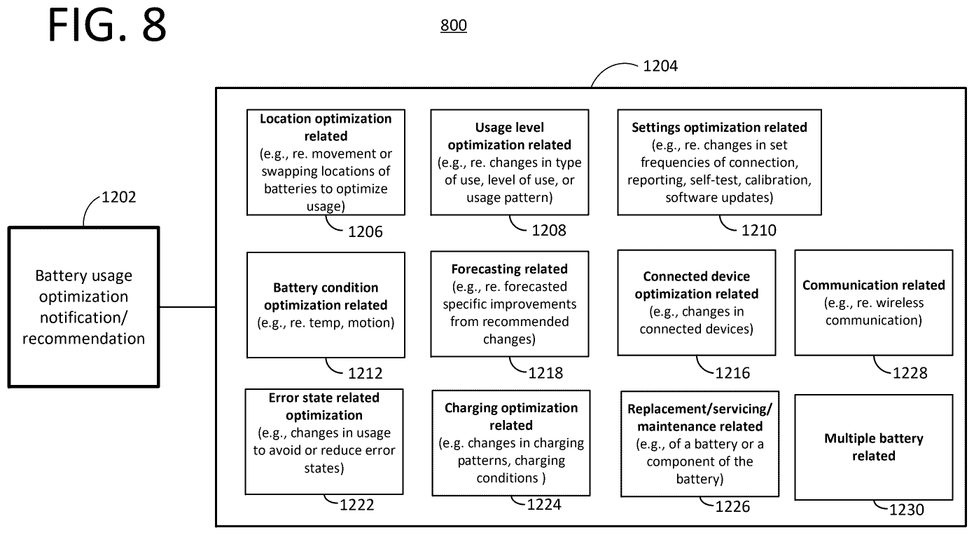

FIG. 8 is a block diagram 800 illustrating example aspects of output of a medical device battery management and optimization system, which may include a battery usage optimization notification/recommendation 1202 (e.g., an optimization notification to recommend an action to optimize battery usage). Various types of recommendations 1204 are described, but it is noted that the categories are not intended to be comprehensive or mutually exclusive and may be overlapping.

Some recommended actions may be related to battery location optimization 1206. This may include, for example, a recommendation to move a battery (which may include a unit including a battery and a connected device) to a different location, or to swap locations of several batteries, examples of which are described in later figures. A change of location may be projected to improve battery usage for many different reasons, including, for example, different projected environmental conditions at a new location (e.g., lower temperature, lower humidity, better wireless signal), and different projected future battery usage patterns at the location (e.g., less projected use, less projected motion or shock).

Some recommended actions may be related to battery usage level changes 1208. For example, this may include a change in the type of use, pattern of use or usage pattern (e.g., different connected device, less projected use, less projected motion or shock, and others).

Some recommended actions may relate to specific battery related settings 1210. For example, this may include a change in wireless connection protocol (e.g., frequency, number of retries per attempt), a change in wireless reporting conditions or frequency (e.g., where the wireless reporting could be initiated or transmitted from a battery or connected device), a change in battery self-test protocol or frequency by a connected device, a change in wireless software updates protocol or frequency, a change in calibration frequency, and others.

Some recommended actions may relate to optimization of specific battery related conditions 1212, such as decreasing battery charging temperature and reducing battery motion or shock.

Some recommended actions may relate to optimization in association with a connected device (e.g., dock or AED) 1216, such as changing the type of connected device that the battery powers or is anticipated to power, or changing to a connected device with a different projected usage pattern, which may impact likely future required power output from the battery.

Some optimization notifications may include a forecasting related component or message 1218, such as a specific projected outcome or result, showing the potential improvement from taking or implementing a recommended action, examples of which are provided herein.

Some recommended actions relate to battery charging 1224, such as recommended actions to reduce rate of capacity decline due to charging patterns or conditions, examples of which are provided herein.

Some recommended actions relate to multiple batteries 1230. For example, some recommendations may be based on one battery's parameters relative to those of other batteries, such as other batteries of a group or user-defined group, other batteries in the same geographic area or a local area, or other batteries of the same type or having other similarities, etc. For example, a recommendation may be based on one battery's parameters being outside of a norm or usual threshold range for the group of batteries as a whole. For example, if it is determined, using historical battery health data, that one battery of a local group is taking unusually long to charge, or is being used unusually often, this could lead to a recommended action to check, service or replace the battery, for example. Additionally, in some examples, such recommended actions may be user configurable, such as by allowing the user to set an amount of deviation from an average that will trigger the recommended action, for example. Furthermore, some recommended actions include actions to be taken with regard to multiple batteries, such as switching the locations, connected devices, or other parameters or conditions of two or more batteries, e.g., for load balancing or other reasons.

Some recommended actions may be error state related 1222, such as actions to reduce the frequency or severity of specific error states.

Some recommended actions may include replacement, calibration, maintenance/repair, calibration or other servicing/conditioning 1226, whether done remotely or at location, of the battery, or a component thereof (e.g., a fuse, sensor, circuit board, electronic component, wireless communication interface, location manager, processor, memory, tags, etc., or any of various other components). Such recommendations may include, for example, warnings or forecasts associated with the recommended action, e.g., a recommended action to replace the battery, or replace a specific sensor, within a certain number of days, weeks or months, for instance, to avoid failure or an error state. As another example, a recommendation to replace a battery may be based on a forecasted amount of time until the battery's capacity drops below a required threshold for usability for the batteries purpose (e.g., for a defibrillator or AED, a certain minimum percentage capacity may be required, such as 10, 20 30, 40, 50, 60, 70 or 80 percent, for example).

Some recommended actions may be communication related 1228. For example, this may include recommendations such as alerts or warnings, such as to check or replace a battery or component such as a battery, battery wireless interface or location manager, based on erroneous, inconsistent, insufficiently frequent, or absent/lack of wireless communications such as periodic historical battery health reporting, location communication or software updating.

Furthermore, in some embodiments, a user may request and obtain a specific type or recommended actions e.g., when a specific battery will need to be replaced, or is forecasted to need to be replaced). Additionally, various types of recommended actions may be user-configurable, in whole or in part, or in various aspects, such as through a battery dashboard. For example, in some embodiments, a user may configure recommendations such that certain recommendations are based on user-set criteria or thresholds. This may include, for example, a user-set minimum capacity or stability level, a user-set remaining battery lifetime warning amount of time, user set criteria for receiving a battery servicing, conditioning or calibration alert, or a set of battery health related criteria relating to a particular recommended action. As another example, a user may set a specific minimum capacity at which the user should be alerted, which may be some amount above a minimum capacity for usability of the battery. As a further example, a user may set a minimum battery charge level below which the user should be alerted. Furthermore, in some embodiments, a battery dashboard may have one or more GUI displays for user configurable recommendation settings. In some cases, thresholds relating to recommendation triggers may be user configurable only within certain ranges, which the user may set using GUI based tools such as dials, for example. Additionally, in some embodiments, a user may create user-defined recommended actions, such as setting a recommended action to be provided when specific user-set battery health criteria are met.

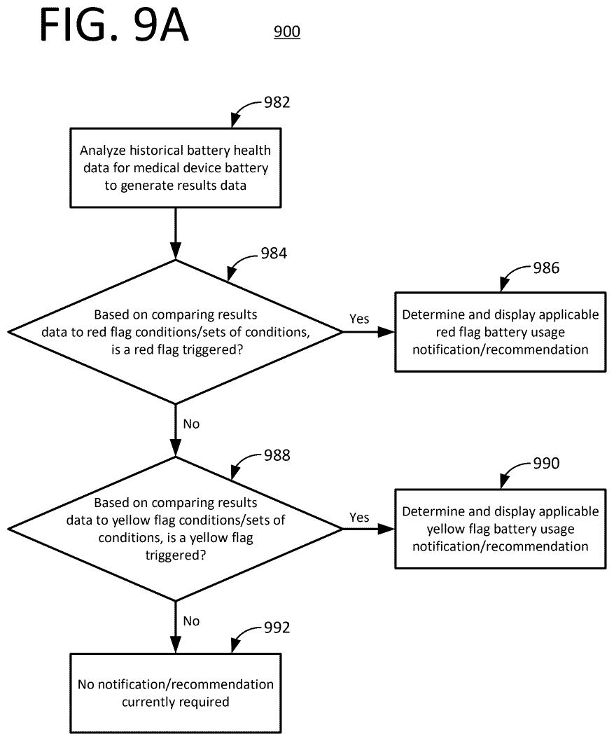

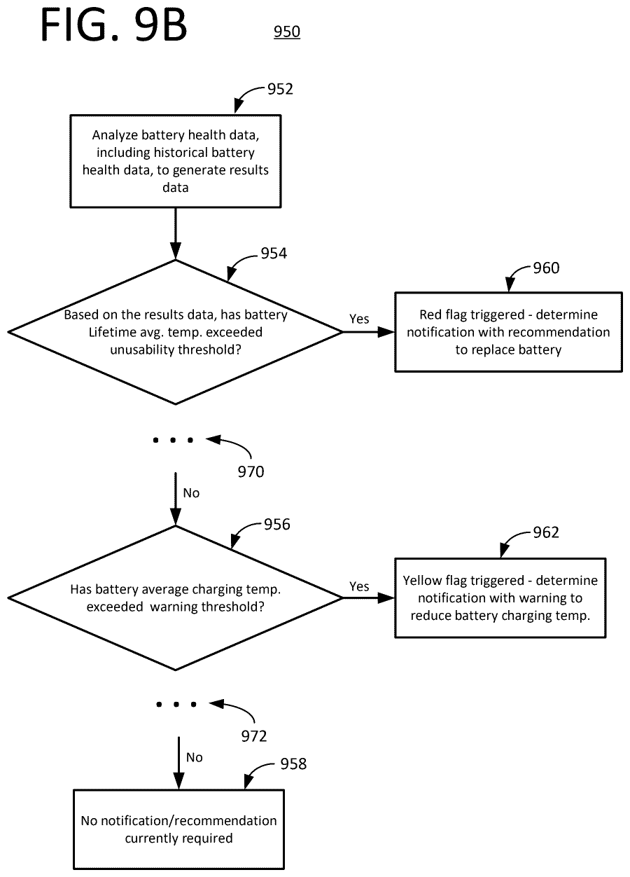

FIG. 9A-B are flow diagram 900, 950 according to an example medical device battery management and optimization methods. In some embodiments, a battery health management and optimization program uses one or more algorithms employing conditional logic used with historical battery health data in determining battery usage notifications/recommendations. For example, multiple sets of one or more conditions may be used, where, if any (or some combination) of the sets of conditions are satisfied, this serves as a trigger to determine and display an appropriate notification/recommendation. Each set of conditions can be a single condition (where, if satisfied, this serves as a trigger), or can be a combination, or Boolean combination, of multiple conditions. For example, a condition could require that condition A and B (or more than two conditions) both be satisfied, or that at least one of condition A or B be satisfied (or more than two conditions), or more complex Boolean strings may be used. Additionally, in some embodiments, certain sets of one or more conditions may trigger more serious, or red flag, notifications/recommendations (e.g., replace a battery or battery component immediately or within a threshold period of time), whereas other sets of conditions may trigger less serious, or yellow flag, conditions (e.g., replace the a battery or component within a period of time greater than a threshold period of time, service or condition the battery or a components, or adjust some specific battery parameter), and more complexity may also be used (e.g., including orange flag conditions, which may have a seriousness less red flag conditions but greater than yellow flag conditions, for example). In various embodiments, logic, such as conditions, specifics, and thresholds applying to red and yellow flag triggering, for example, may be automatic (e.g., set without the possibility of user interaction), user-configurable, or some combination of both.

In FIG. 9A, at step 982, for a medical device battery, a computing device(s) analyzes historical battery health data to generate results data (e.g., this may include generated data sets or plots representative of battery health parameters over time). The medical device battery may be among a group of medical device batteries belonging to a user-defined grouping, about which the user has made a request to access data.

At step 984, based on comparing the results data to one or more red flag conditions, or a set of such one or more conditions, as described above, the computing device(s) determines whether a red flag is triggered. If so, then, at step 986, the computing device(s) determines and displaying to a user an appropriate red flag notification/recommendation (e.g., a warning to replace the battery, or one or more other recommended actions). If not, method proceeds to step 988.

At step 988, based on comparing the results data to one or more yellow flag conditions, or a set of such one or more conditions, as described above, the computing device determines whether a yellow flag is triggered. If so, then, at step 990, the computing device(s) determines and displaying to a user an appropriate yellow flag notification/recommendation (e.g., adjust a specific battery parameter).

If not, then, at step 992, the computing device determines that no notification/recommendation is currently required.

FIG. 9B provides an example of a method 950 providing as simplified example logical flow corresponding to FIG. 9A. At step 952, a remote computing device analyzes historical battery health data to generate results data (e.g., this may include generated data sets or plots representative of battery health parameters over time, which may include a plot representing battery lifetime temperature data over a period of time).

At step 954, based on the results data, the computing device determines whether the battery lifetime average temperature has exceeded a specific unusability threshold (e.g., a specified temperature above which the battery is identifiable as unusable), which represents a red flag trigger. If so, then, at step 960, the computing device(s) determines and displaying to a user an appropriate red flag notification/recommendation (e.g., a warning to replace the battery).

As represented by ellipsis 970, in some embodiments, other red flag sets of one or more conditions may be included, in various Boolean combinations, as described above. If no red flag is triggered, then the method 950 proceeds to step 956.

At step 956, the computing device determines whether the battery average charging temperature has exceeded a specified warning threshold (e.g., a specific temperature, above with the warning is triggered). If so, then, at step 962, the computing device(s) determines and displaying to a user an appropriate red flag notification/recommendation (e.g., a warning to reduce the battery charging temperature). As described with reference to later figures, in some embodiments, notifications may also include addition information, which may be based on historical battery health data, such as a forecast including a specific improvement projected to result from taking a specific recommended action (e.g., a projected increase in remaining battery lifespan resulting from reducing the future average battery charging temperature by a specified number of degrees).

As represented by ellipsis 972, in some embodiments, other yellow flag sets of one or more conditions may be included, in various Boolean combinations, as described above. If no yellow flag is triggered, then the method 950 proceeds to step 958, at which the computing device(s) determines that no notification/recommendation is currently required.

FIG. 10 is a mixed diagram illustrating examples aspects 1000 of medical device management and optimization system analysis, including generation and use of historical battery parameter plots. At block 1004, a computing device(s) generates battery parameter plots, e.g., tracking each parameter over a past period of time. In the example shown, plots 1002 are generated for parameters including wireless signal level 1018, wireless reporting frequency setting 1020, average number of wireless connection retries per connection attempt 1022, overall power output 1024, and capacity 1026, over a past time period including, in chronological order, times T1-T4.

Herein, parameters may be specified as high, medium or low. High indicates that the value of the parameter is within a high range defined by a lower threshold value and an upper threshold value. Medium indicates that the value of the parameter is within a medium range defined by a lower threshold value and an upper threshold value, where the entire medium range is lower than the entire high range. Low indicates that the value of the parameter is within a low range defined by a lower threshold value and an upper threshold value, wherein the entire low range is lower than the entire medium range. In some embodiments, the thresholds and ranges may be dynamic and vary or change based on parameters or conditions such as, e.g., battery type, battery parameters, historical battery usage, or other conditions. Additionally, in some embodiments, the thresholds or ranges may be configurable or settable, such as by a user.

As can be seen in plot 1018, wireless signal remained high (e.g., within a high range defined by a lower and an upper threshold signal level) from times T1 to T3, but sharply dropped from time T3 to time T4 to low (e.g., within a low range defined by a lower and an upper threshold signal level). From other historical battery health data, it is determined that the sharp drop at time T3 corresponds in time with a change of location of the medical device battery from location A to location B.

In plot 1020, it can be seen that wireless reporting frequency setting (e.g., where the wireless reporting could be initiated or transmitted from a battery or connected device) is set at low (e.g., within a low range defined by a lower threshold setting and an upper threshold setting) from time T1-T2, between low and medium (where the medium range is defined by a lower threshold setting and an upper threshold setting) from time T2 to time T3, and sharply increases to high (e.g., within a high range defined by a lower threshold setting and an upper threshold setting) from time T3 to time T4. The sharp setting increase at time T3 also corresponds with the change of location from Location A to Location B.

In plot 1022, it can be seen that the average number of wireless connection retries per connection attempt was low (e.g., within a low range defined by a lower threshold average number and an upper threshold average number) from time T1 to time T2, medium (e.g., within a medium range defined by a lower threshold average number and an upper threshold average number) from time T2 to time T3, and high (e.g., within a high range defined by a lower threshold average number and an upper threshold average number) from time T3 to time T4.

Plot 1024 shows that overall battery power output was low (e.g., within a low range defined by a lower threshold power output and an upper threshold power output) from time T1 to time T2, medium (e.g., within a medium range defined by a lower threshold power output and an upper threshold power output) from time T2 to time T3, and high (e.g., within a high range defined by a lower threshold power output and an upper threshold power output) from time T3 to time T4.

Finally, plot 1026 shows that battery capacity declined at a low rate (e.g., in a low range defined by an upper threshold rate and a lower threshold rate) from time T1 to time T2, a medium rate (e.g., in a medium range defined by an upper threshold rate and a lower threshold rate) from time T2 to time T3, and a high rate (e.g., in a high range defined by an upper threshold rate and a lower threshold rate) from time T3 to time T4.

In some embodiments, the plots may be generated using historical battery health data obtained from periodic transmissions from the battery (or unit with the battery), using data stored in a medical device database, and based on one or more algorithms of a battery management and optimization program executed by a computing device(s). Furthermore, in some embodiments, the computing device(s) may use one or more algorithms to analyze the plotted data in determining a recommended action for optimization of battery usage. While visibly shown as plots in FIG. 10, in some embodiments, the visible plots would not actually be generated, but data sets representing the chronologically tracked parameter data of the plots would be generated, stored and used. While FIG. 10 shows a relatively simple example, in some embodiments, more complex plots may be used, which could include, for example, multidimensional plots, plots relating to multiple parameters, plots generated based on one or more mathematically, functionally or algorithmically modified plots of tracked parameter data, plots generated using regression or other algorithms, plots generated using one or more mathematical models, or using artificial intelligence or neural networks, or in other ways. For example, in some embodiments, a regression algorithm may be used in analyzing plots of multiple battery parameters over time (e.g., power output, charging temperatures) relative to a different parameter being focused on (e.g., rate of capacity decline), to make predictions about how changing one or more of the multiple battery parameters (e.g., reducing battery power output charging temperature) may influence another parameter being focused on (rate of capacity decline) (e.g., parameter plots, such as plots 1002 in FIG. 10). This, in turn, may be used in making forecasts and determining recommended actions to optimize battery usage (e.g., recommendations relating to decreasing battery temperature by a specific amount along with a prediction of the forecasted improvement in terms of reduced rate of battery capacity decline).

In some embodiments, the data from the plots is algorithmically analyzed, individually and/or together, to determine relevant information and to determine a recommended action. For example, it taken into account that, from plot 1018 that location B appears to have significantly lower overall wireless signal level than location A.

Overall, via algorithmic analysis of the plots in total, the computing device(s) may make various determinations. Specifically, for example, it may determine that is it likely that the lower wireless signal at new location B contributed to a greater number of wireless connection retries per connection attempt being required, which led to a higher overall battery power output, which led to a higher overall power output and a higher rate of battery capacity decline. It may also determine that it is likely that increasing the wireless reporting frequency setting led to higher overall power output and a higher rate of battery capacity decline.

In the embodiment shown, the computing device(s) then algorithmically determines multiple candidate potential recommended actions to evaluate to determine the action to recommend. This may include, for example, apply a set of optimization criteria, along with using forecasting. For example, the optimization criteria could provide a weighting of the positive or negative value, from a battery usage optimization perspective, of various changes, taking into account a range of optimization criteria and weightings (which, in some embodiments may be entirely or partially user-configurable). In various embodiments, one or more functions or models may be used in this regard. Furthermore, in various embodiments, various algorithms, mathematical models, or artificial intelligence models along with weightings for each of various factors, may be determined algorithmically by the computing device, such as based on stored optimization criteria, or an administrative user may provide or adjust factors or weighting, or a combination of both, or in other ways.

In the example, shown, four potential recommendations are generated. One potential recommended action includes changing the battery location from location A to location C 1012 (at location C, it is determined from historical battery health data that wireless signal level has been, and is forecasted to remain, high). Another potential recommended action includes reducing the reporting frequency setting from high to medium. A third potential recommended action includes reducing a maximum connection retries per connection attempt setting from the current setting of 15 to a new setting of 3. A fourth third potential recommended action includes reducing a maximum connection retries per connection attempt setting from the current setting of 15 to a new setting of 2. Furthermore, in the example, shown, based on optimization criteria, each of the potential recommended actions is algorithmically numerically scored, e.g., on a scale of 0-100, in terms of the magnitude of optimization value each is determined or forecasted to provide, if implemented. In this regard, the reducing maximum retries from 15 to 3 option 1016 scored highest, with a score of 76, the reducing maximum retries from 15 to 2 option 1030 scored second highest, with a score of 71, the changing location option 1012 scored third highest, with a score of 62, and the reduction of reporting frequency setting 1014 scored the lowest, with a score of 34. As one example, the option of reducing the maximum number of retires from 15 to 2 option may have scored lower than the option of reducing the maximum number of retires from 15 to 3 because, according to specified optimization criteria, although battery lifespan is projected to be slightly increased by the option, that advantage was outweighed by the disadvantage related to a projected decrease in total connections and associated data transmissions (e.g., leading to less comprehensive collected data), leading to the slightly lower score. For example, in some embodiments, plots of each of multiple battery parameters (e.g., plots 1018-1026) may be analyzed for a recent period of time, or a moving average may be calculated. Output from such analysis could be used as input in determining forecasts relating to improvements that may result from specific parameter changes, which may, in turn, but used in scoring relating to such potential changes or adjustments.

Since the reduction of maximum retries from 15 to 3 option 1016 scored highest, it is selected as the recommended action to provide. Based on that, block 1018 shows an example optimization notification that may be determined and displayed, recommending reducing the maximum number of connection retries per attempt from 15 to 3, and providing a projected resulting improvement of an increase in remaining battery lifespan from 2.3 years to 3.1 years. For example, the increase in battery lifespan may be determined based on a projected reduction in capacity decline, and could also be based on a certain minimum capacity considered to be the limit of the useful life of the battery, in general or taking into account anticipated battery use, for example (e.g., a battery for powering an AED may require a relatively high minimum capacity, in order to be able to provide sufficient power for defibrillation shocks or for other uses). In some embodiments, a recommended action may include a range of values from which the user may select, or may provide a specific value but allow the user to modify or adjust the value. Furthermore, in some embodiments, if the user chooses to implement the action, the user may make an appropriate selection on the display/GUI, and the remote computing device(s) may implement the action, such as by communicating with the one or more batteries to send one or more commands to make the appropriate changes or adjustments, if appropriate.

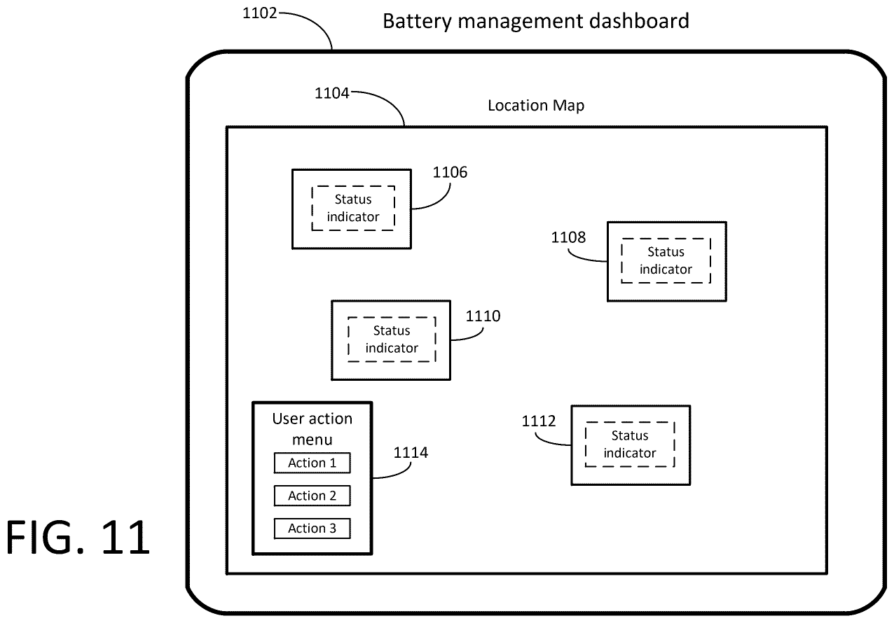

FIGS. 11-20 illustrate example simplified medical device battery management and optimization dashboards. Various of FIGS. 11-20 include summary data that may be generated and transmitted, for example, in response to a request (e.g., via one or more selections or entries via a display/GUI), relating to a user-defined grouping of medical device batteries (e.g., batteries or units at a specified one or more locations, or specified units or batteries). For example, recommended actions to optimize battery usage (for one or more batteries or other devices) may be forecasted to result in increased battery lifespan, reduced rate of capacity decrease, reduced risk rate of reliability decline, more efficient battery use of multiple batteries (e.g., load balancing between batteries), or less costs associated with batteries or battery use (e.g., less or later battery replacement costs, less or later maintenance costs), among other things.

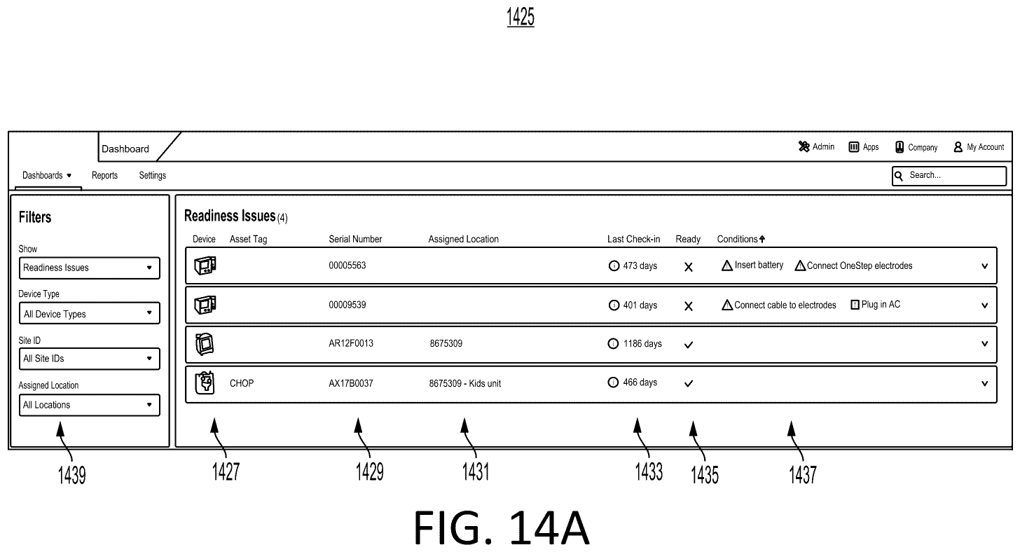

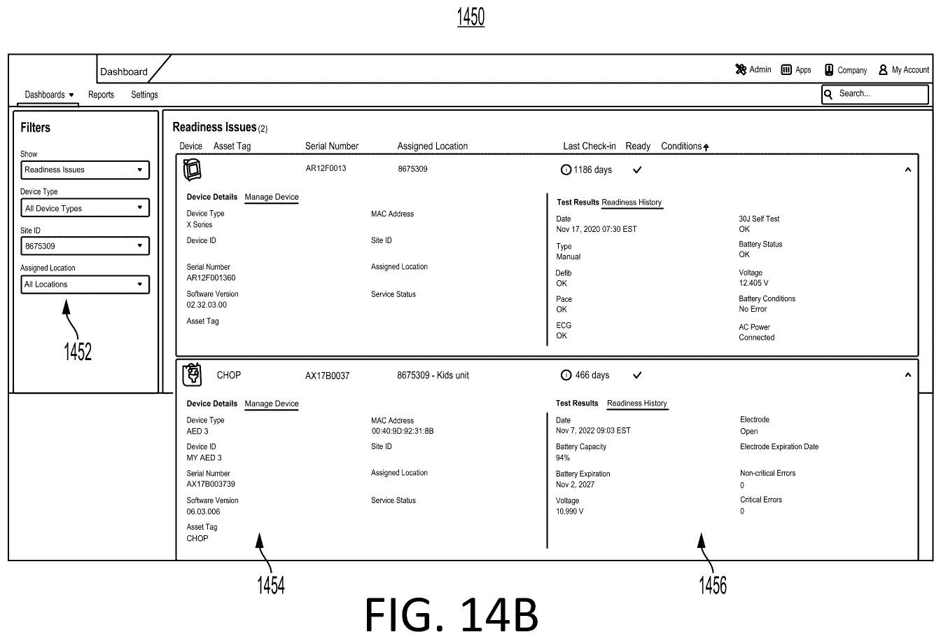

FIG. 11 shows a example dashboard 1102, including a location map 1104. The location map 1104 includes display indicators 1106-1112 corresponding with each of multiple batteries or units, and/or, in some embodiments, devices (e.g., medical devices such as AEDs other than batteries or units). Examples of icons 1427 that may be used as device indicators are provided, for example, in FIGS. 14A-B. The locations of each of the display indicators 1106-1112 on the map may correspond, generally or roughly, to the locations of each of the batteries or units, e.g., geographically or within a building, and/or according to user-defined grouping or region. Each of the displayed indicators 1106-1112 includes a status indicator with battery health status information relating to each battery or unit (e.g., battery health information, such as capacity of each battery). The dashboard 1102 also includes a user action menu 1114. In various examples, the user action menu 1114 may allow the administrative user to implement an action, or to select the action to obtain additional information, or to modify the action and then implement the modified action. The actions may relate to medical device battery management and optimization, and may, in some examples, include optimization notifications with recommended actions to optimize battery usage.

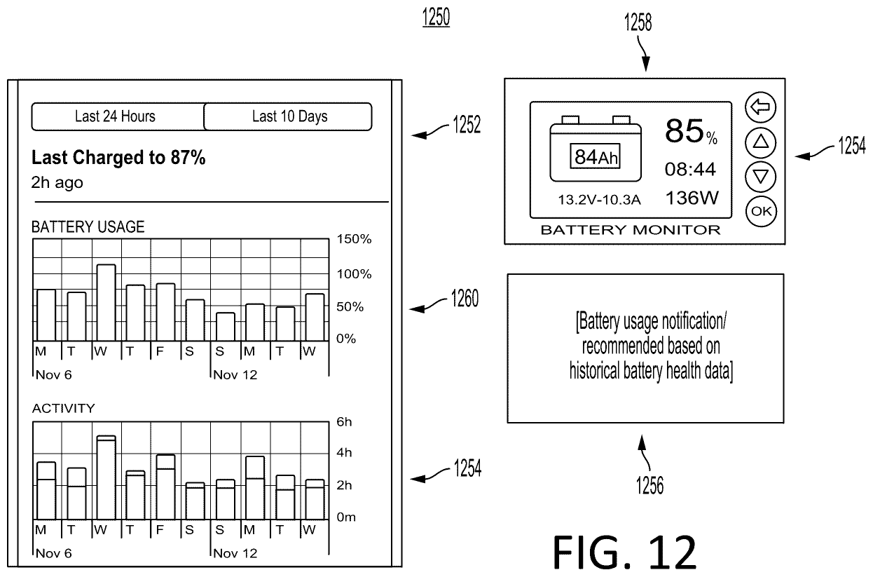

FIG. 12 provides example display areas of a dashboard 1250 according to some embodiments. As depicted, the dashboard 1250 includes display area 1254, includes current battery health relating information, including, as shown, the current time (e.g., 8:44 am), battery voltage (13.2V), battery current (10.3 A), battery power output (136 W), as well as GUI navigation buttons 1258. The dashboard 1250 also includes historical battery health information, including, as shown, for the last 10 days 1252, information including the last charge level after charging (87%), battery usage level over the last 10 days 1260 (November 6-November 15), and battery activity level, in time used, over the last 10 days 1254. The dashboard 1250 also includes a battery usage optimization notification/recommendation based on historical battery health data 1256, examples of which are provided herein.

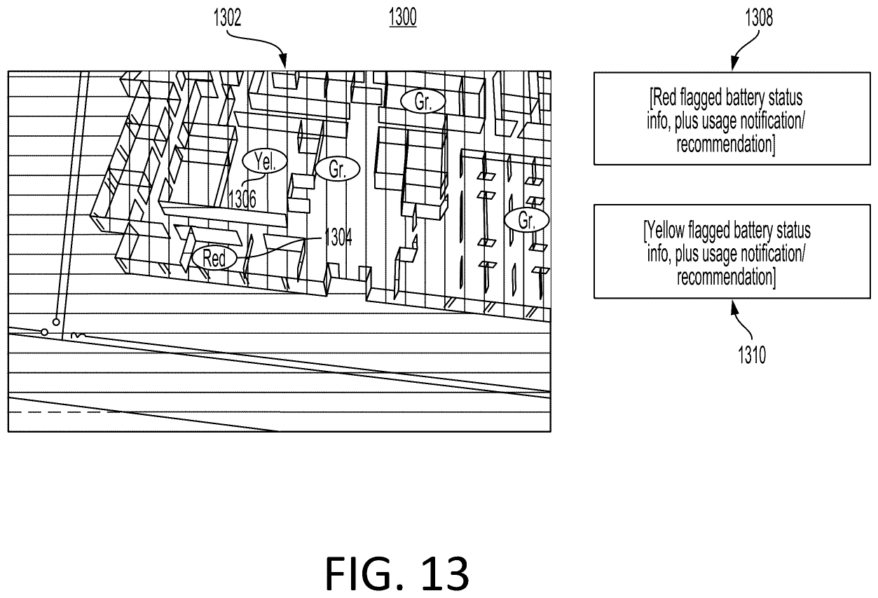

FIG. 13 provides an example portion of a display 1302 of a dashboard 1300 including a display 1302 representing a three-dimensional map of a floor of a building, including various walls defining rooms. The display includes multiple icons, displayed in colors, including a red icon 1304, a yellow icon 1306, and three green icons, where each icon represents a medical device battery in the location/room shown by the position of the icon. In the embodiment shown, a notification 1308, including battery status information and a red flagged notification/recommendation, is shown for the battery represented by the red icon 1304, and a notification 1310 including a yellow flagged notification/recommendation, is shown for the battery represented by the yellow icon 1306, which may include recommended actions determined by a computing device(s) using historical battery usage data, for example. Examples of such potential such notifications/recommendations are provided herein. In some embodiments, the recommendations may be displayed on a computer or handheld of an administrative user, and may provide the user with an option to cause the recommended action to be implemented on the battery or unit (e.g., a change of a parameter or setting of the battery or unit), such as based on the recommendation or other provided information (e.g., other battery health information or a forecast relating to improvement that may result from taking a recommended action).

FIG. 14A illustrates an example battery management dashboard 1425 including medical readiness information and including battery related information. As shown, the dashboard 1425 includes information relating to several devices, as indicated by icons 1427, which may, in various examples, include batteries and/or units (e.g., with connected devices such as defibrillators or battery docks), or other devices. In some embodiments, the dashboards 1425, 1450 of FIGS. 14A-B, may be displayed in a computing device (e.g., computer or handheld) of a user or administrative user, along with a recommended action that the user may take to optimize battery usage. In some examples, the user may then choose to implement (or not implement) a recommended action, such as based on displayed information, and, if the user chooses to implement the action, a remote computing device may transmit a command to the battery or unit, accordingly, to implement the change or adjustment, for example.

As shown by display portion 1439, “All Site IDs” and “All Locations” are selected. The site ID may be a location of the device (e.g., a specific hospital) and the assigned location may be a sub-location at the location (e.g., a specific unit, area or room within the specific hospital, or a specific city within a county, as examples). Since “All Site IDs” and “All Locations” are selected, all devices associated with all site IDs and all assigned locations are shown. For two of the devices, in column 1431, no site ID or assigned location is indicated, which may mean that the device is not associated with any site ID or assigned location. However, for device serial number AR12F0013, “8675309” is indicated, which represents the Site ID, although no specific assigned location is indicated, and for device serial number AX17B0037, “8675309—Kids unit” is indicated, which represents the Site ID of 8675309 and a specified assigned location of “Kids unit” (such as a pediatric unit, which may be, e.g., a specific area, or one or more rooms, at a specific hospital associated with site ID 8675309, for example).

Column 1433 indicates how many days have passed since the last check in by the device (e.g., battery or unit). For example, this column may indicate, for example, how many days or hours have passed since the battery or unit last transmitted historical battery health data to a remote computing device, which transmitted data may be accessible via the dashboard 1425, in some embodiments, along with battery usage optimization notifications/recommendations, for example.

Column 1435 shows an overall readiness of the device (e.g., in this case, for defibrillation, but, in other embodiments, this may relate to, e.g., battery or unit charge level or readiness, for example), with a check mark indicating that the device is ready or an X indicating that the device is not ready. Column 1437 includes information about each of the devices, including battery related information—specifically, for device serial number 00005563 (as indicated in display portion 1429), a message is provided that the battery needs to be inserted, and, for device serial number AX17B0037, a message is provided that the device should be plugged into AC power, which may mean that the battery of the device is not connected or has no or insufficient charge remaining, for example. In some embodiments, the devices may be batteries or units, and the messages may relate to the batteries or units-e.g., that a specific battery is not ready because it is not charged, or that another specific battery is ready because it is fully or sufficiently charged, for example.

FIG. 14B illustrates an example battery management dashboard 1450 including device readiness and battery health information. As shown in display portion 1452, in the “Site ID” field, site ID 8675309 is selected. Site ID 8675309 may represent a location, such as may be established or specified by a user, where a number of devices, such as may batteries or units, are assigned to and located, and may represent a user-defined grouping of devices (e.g., batteries or units, or other devices). In this case, since Site ID 8675309 is selected, this may serve as a user request relating to the user-defined grouping, and an example of summary data regarding the two devices associated with this site ID is shown, including data in display portions 1454 and 1456 (which represent two of the four devices shown in FIG. 14A).

Display portion 1454 provides various information about each device (e.g., type, software version included, and other information). In some examples, the devices may be batteries or units, as described herein, and summary data may be provided that relates to the batteries and units (e.g., battery serial number, type of battery, connected device associated with the battery, and other information), and/or other data, such as battery health data.

Display portion 1456 provides further information about each device. In some examples, the devices may be batteries or units, as described herein, and summary data may be provided that relates to the batteries and units, including, e.g., battery status, battery capacity, battery voltage, and battery error conditions, and/or other data, such as battery health data or historical battery health data.

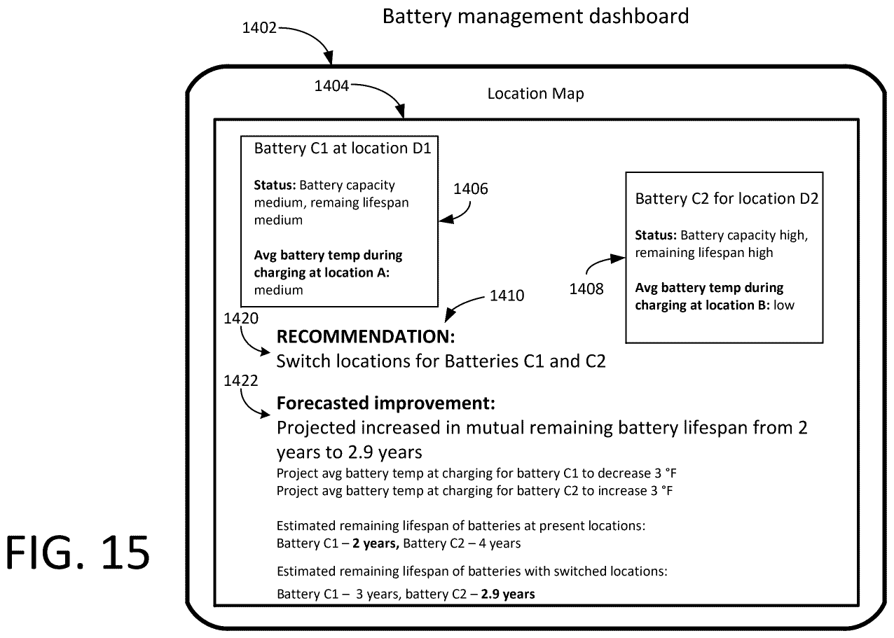

In some examples, battery use notifications/recommendations may also be provided, such as in addition to or along with summary data, such as on the dashboards 1425, 1450 and related to batteries or units, examples. Examples of battery usage optimization notifications/recommendations are provided with reference to various figures herein, including, for example, FIGS. 15-21. In some embodiments, data, such as event data, associated with displayed notifications/recommendations, as well as implemented actions based on recommendations, may be stored, such as in a medical device database. For example, a battery or unit may store such data, and include it in data periodically transmitted (e.g., by batteries or connected devices) to a remote computing device for storage in a medical device database, or a remote computing device may directly store the data in a medical device database. In some embodiments, such data could include, e.g., event flag data to signal that a specific notification/recommendation has been sent, and/or that a specific recommended action has been taken or noted, for example. This, in turn, may be used to track recommendations/notifications that have been sent and/or actions that have been taken, such as to track implemented changes in battery parameters or usage, and as a management or quality control check to ensure that such recommendations have been provided and that such actions have been implemented, for example.

In FIG. 15, example dashboard 1402 includes a location map 1404 with display indicators 1406, 1408 relating to each of two medical device batteries C1, C2. Display indicator 1406, which relates to Battery C1 at location D1, indicates status as battery capacity medium (e.g., in a medium range defined by an upper threshold capacity and a lower threshold capacity), with the battery estimated to have a medium remaining lifespan (e.g., in a medium range defined by an upper threshold remaining lifespan and a lower threshold remaining lifespan), and also indicates that the average temperature of the battery at location 1 is medium (e.g., in a medium range defined by an upper threshold average temperature and a lower threshold average temperature). For battery C2 at location D2, display indicator 1408 estimates the battery capacity at high (e.g., in a high range defined by an upper threshold capacity and a lower threshold capacity), with the battery estimated to have high remaining lifespan (e.g., in a high range defined by an upper threshold remaining lifespan and a lower threshold remaining lifespan), and that the average battery temperature at charging is low (e.g., in a low range defined by an upper threshold average temperature and a lower threshold average temperature) (e.g., a reason could be that an average ambient temperature at location D2 is lower than at location D1, leading to a lower average charging temperature at location D2 than at location D1, as one example).

The dashboard 1402 also includes an optimization notification 1410 including a recommendation 1420 to switch locations for batteries 1 and 2. For example, a computing device(s), using stored historical battery health data transmitted by batteries C1 and C2, and using a battery health management and optimization program, may have analyzed the data and determined to provide this recommended action. While not shown, in some embodiments, the user may be provided with an option to obtain more information, such as more specifics on the recommendation or how to implement it. The optimization notification 1410 also includes an improvement projected or estimated to be likely to result 1422, should the battery locations be switched. Specifically, a message is included that indicates a projected increase in mutual remaining battery lifespan from 2 years to 2.9 years. Addition information is provided, indicating that, if the action is taken, it is projected that the average battery temperature at charging for battery C1 will decrease 3° F., but the average battery temperature for battery C2 will increase 3° F. Furthermore, it is projected that, without the change in locations, battery C1 has an estimated remaining lifespan of 2 years, and battery C2 has an estimated remaining lifespan of 4 years, but with the change in locations, battery C1 has an estimated remaining lifespan of 3 years, and battery C2 has an estimated remaining lifespan of 2.9 years. As such, with the switch in locations, although the lifespan of battery C2 is projected to decrease, with the increase in the lifespan of battery 1, the lifespans of both batteries together will increase with the change. This may be determined to be optimal, providing mor optimal load balancing and efficient usage pattern for the two batteries, considered together. In various examples, many factors, which may be reflected in historical battery health data, may lead to a recommended action to switch battery locations, including, among others, battery location, battery environment conditions, battery capacity, battery age, battery error states, battery impedance, battery power output, battery charging, and a connected device to be powered by the medical device battery. In some embodiments, the recommendation 1410 may be displayed on a computer or handheld of a user, such as an administrator, and may offer the user an option related to implementing the recommended action. For example, here, the user may be provided with a displayed option to store a note or order to cause the battery locations to be switched.

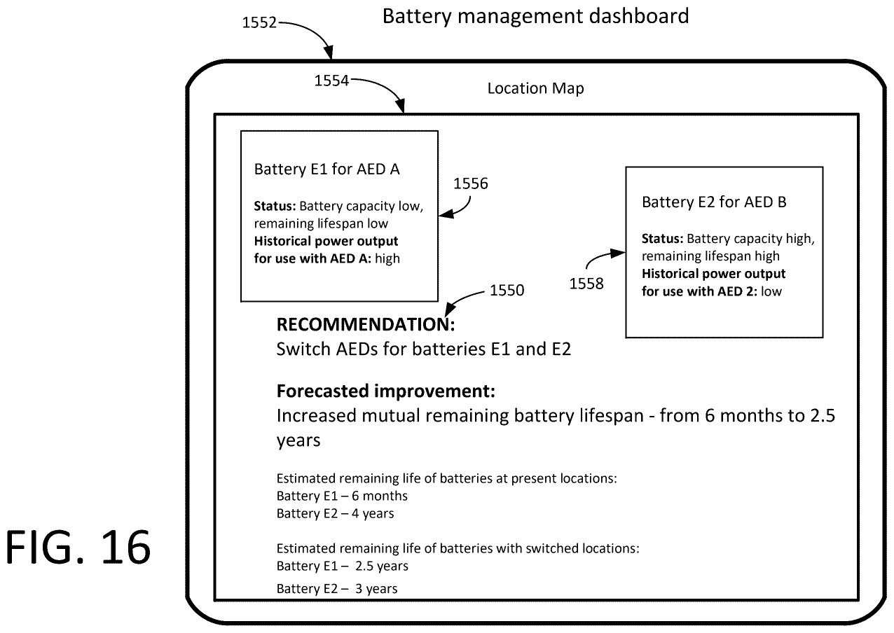

FIG. 16 provides an example dashboard 1552 relating to batteries that are connected with AEDs in units as described herein. The example battery management dashboard 1552 includes a location map 1554 with indicators 1556, 1558. Display indicator 1556, which relates to Battery E1 for AED A, indicates status as battery capacity low (e.g., in a low range defined by an upper threshold capacity and a lower threshold capacity), with the battery estimated to have a low remaining lifespan (e.g., in a low range defined by an upper threshold remaining lifespan and a lower threshold remaining lifespan) and historical battery power output for use with AED A as high (e.g., in a high range defined by an upper threshold power output and a lower threshold power output) (where battery E1 is to power AED A). Display indicator 1558, which relates to Battery E2 for AED B, indicates status as battery capacity high (e.g., in a high range defined by an upper threshold capacity and a lower threshold capacity), with the battery estimated to have a high remaining lifespan (e.g., in a high range defined by an upper threshold remaining lifespan and a lower threshold remaining lifespan), and historical battery power output for use with AED A as low (e.g., in a low range defined by an upper threshold power output and a lower threshold power output). In this example, the computing device(s), based on historical battery health data provided by the units, determines an optimization notification including a recommendation to switch the AEDs for connection and use with each of batteries E1 and E2. If this action is taken, it is forecasted to improve mutual remaining battery lifespan from 6 months to 2.5 years. Specifically, the estimated remaining life of battery E1 is projected to increase from 6 months to 2.5 years, although the estimated remaining life of battery E2 is projected to decrease from 4 years to 3 years. This may be determined to be more optimal, providing a more balanced and efficient usage pattern for the two batteries, considered together. A key factor in the determination of this recommended action is that, historically, power output required from a battery used with AED A is much higher than AED B, and so, for balance and highest mutual battery lifespan, it is more optimal to use battery 2 for AED A, since battery 2 has a much higher remaining capacity than battery 1. In some embodiments, the recommendation 1550 may be displayed on a computer or handheld of a user, such as an administrator, and may offer the user an option related to implementing or not implementing the recommended action, such as based on displayed information. For example, here, the user may be provided with a displayed option to store a note or order relating to the batteries to be switched.

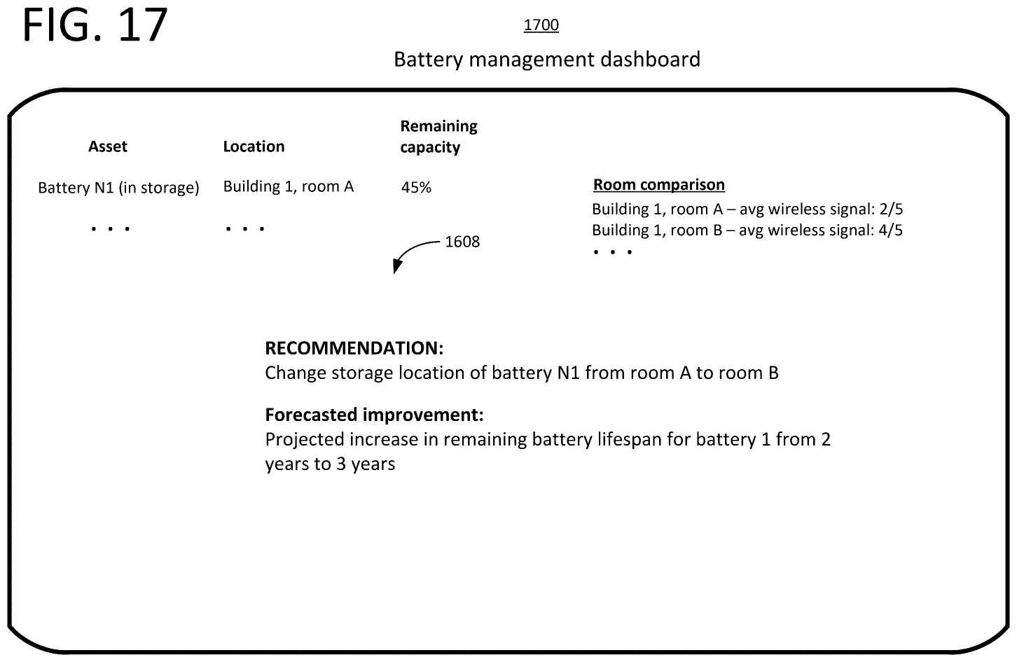

FIG. 17 provides an example dashboard 1700 that provides summary data for a group of batteries, including battery 1, which is indicated as being stored in building 1, room A, and having a remaining capacity of 45% (of its absolute capacity, which is its capacity when new). Additionally, room comparison information is provided, relating to building 1, indicating that room A has an average wireless signal level of 2 out of 5, whereas room B has a much higher average wireless signal level of 4 out of 5. Based on historical battery health data, a computing device(s) determines an optimization notification including a recommendation to change the storage location of battery N1 from room A to room B, within the building. This action, if implemented, is forecasted to result in an increase in remaining battery lifespan for battery N1 from 2 years to 3 years. This may be, for example, because it is determined that the better signal level at location B will lead to less wireless connection attempts, and/or faster data transfer rates, leading to less power output being required from the battery relating to wireless communication, such as for wireless reporting of historical battery health data (e.g., where the wireless reporting could be initiated or transmitted from a battery or connected device), software updates, or other reasons. In various examples, many different battery health related factors may be taken into account in determining a recommended change in battery location, including, for example, battery wireless communication settings or protocol, battery motion or shock, battery environment conditions (based on location), battery capacity, battery age, battery error states, battery impedance, battery power output, battery charging, a connected device to be powered by the medical device battery. For example, if various of these conditions are forecasted, from historical battery health data, to be different between locations (e.g., less battery shock historically occurs at one location than another location), this could impact, for example, remaining battery lifespan and/or battery stability, which may factor into determination of a recommended action. In some embodiments, the recommendation 1608 may be displayed on a computer or handheld of a user, such as an administrator, and may offer the user an option related to implementing the recommended action, such as, e.g., an option to store a note or order to have the battery moved from room A to room B.

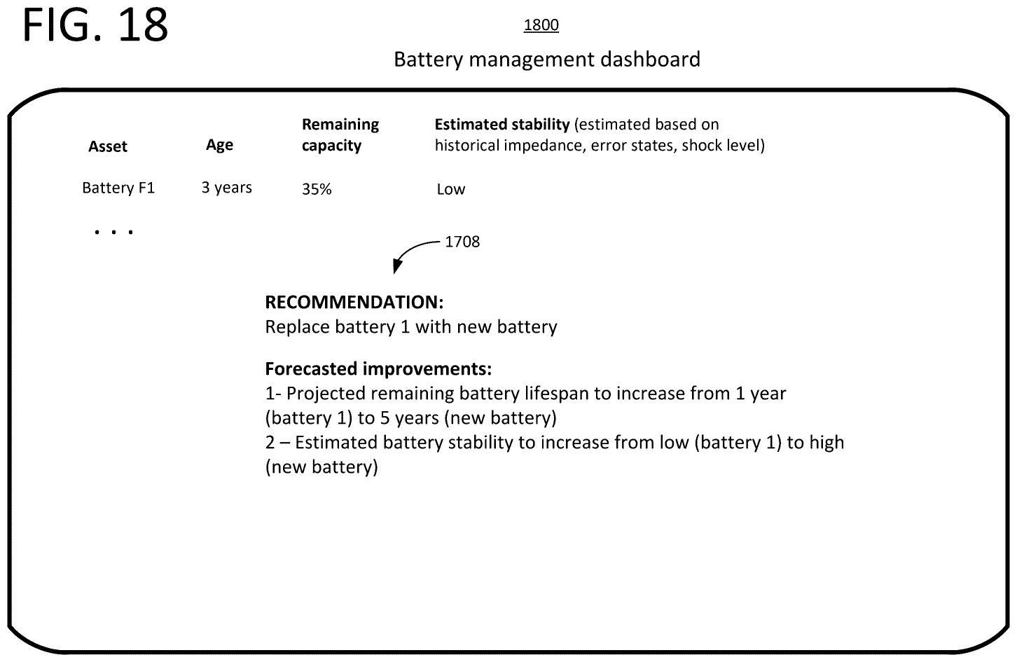

FIG. 18 provides an example dashboard 1800 that provides summary information for a group of batteries, including battery F1, which is indicated as being 3 years old, have a remaining capacity of 35% (of its absolute capacity), and its stability is estimated as low (e.g., in a low range defined by an upper threshold stability value and a lower threshold stability value) (e.g., based at least in part on historical battery health data on impedance, battery error states that have occurred, and overall historical battery shock level). As described above, a low stability may be considered to be associated with a greater risk of battery error or failure. Based on the historical battery health data, a computing device(s) determines that a recommended action 1708 to replace battery 1 with a new battery. If this action is taken, is forecasted to improve battery lifespan from 1 year (for the current battery) to 5 years (for a new battery), and to improve battery stability from low (for the current battery) to high (for a new battery) (e.g., in a high range defined by an upper threshold stability value and a lower threshold stability value). In some embodiments, the recommendation 1708 may be displayed on a computer or handheld of a user, such as an administrator, and may offer the user an option related to implementing the recommended action, such as, e.g., an option to store a note or order to have battery 1 replaced with a new battery.

Many factors may be taken into account in determining a battery replacement recommendation, including, for example, among others, battery capacity, battery age, battery error states, battery impedance, battery power output, battery charging, and a connected device to be powered by the medical device battery. For example, a forecast may be made of projected battery use (e.g., depending on the connected device, such as an AED, and it's forecasted amount of use), and this forecast may be taken into account in determining whether battery replacement is optimal (e.g., if the current battery may soon be unable to power the AED, if needed, or if its stability is unacceptably low). In various examples, optimization criteria may be used that include various other factors, such as, for example, battery replacement cost.

In other examples, recommended actions may alternatively include replacement or repair of specific battery or unit components, such as sensors, for example. Taking such recommended actions, among others, may effectively increase the remaining lifespan of a medical device battery substantially, such as from a total lifespan of, for example, 5 years, to a total lifespan of, for example, 6-10 years or more.

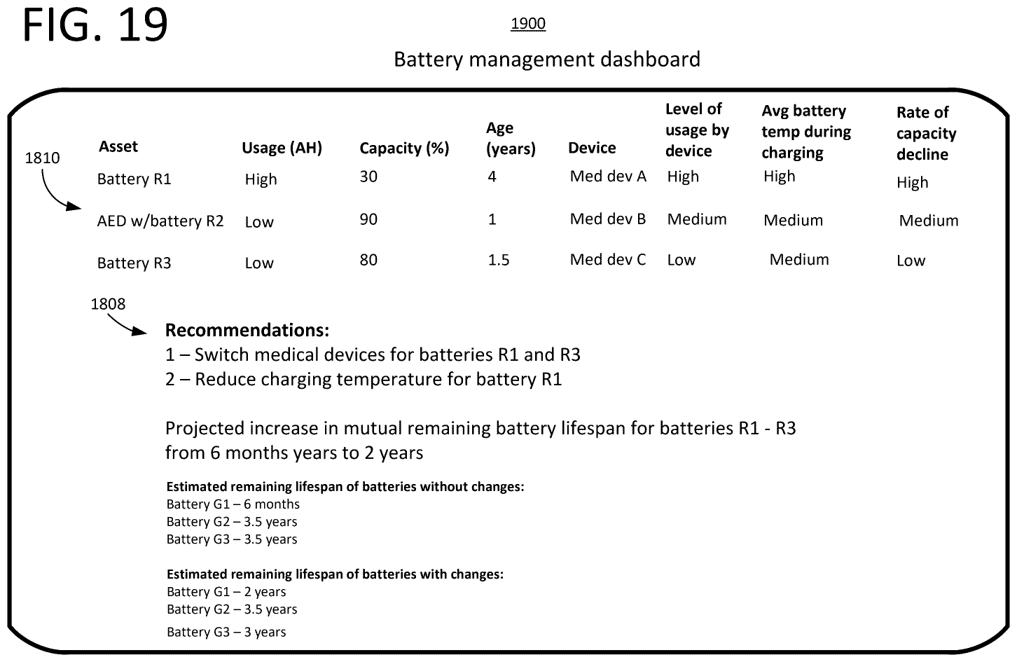

FIG. 19 provides an example of a dashboard 1900 in which an optimization notification is provided that relates to three batteries. Dashboard 1800 provides a list of batteries 1810, which may be, for example, batteries of a user-defined grouping, including, e.g., some with may be separate from any connected device, and some with may be connected to a connected device. Specifically, based on data including historical battery health data, battery R1 is indicated has having high usage (e.g., in a high range defined by an upper threshold usage level and a lower threshold usage level), 30% capacity, age of 4 years, used to power medical device A, which has a high level of usage (e.g., in a high range defined by an upper threshold usage level and a lower threshold usage level), having a high average battery temperature during charging (e.g., in a high range defined by an upper threshold temperature and a lower threshold temperature), and having a high rate of capacity decline (e.g., in a high range defined by an upper threshold rate and a lower threshold rate). Battery R2 is indicated has having low usage (e.g., in a low range defined by an upper threshold usage level and a lower threshold usage level), 90% capacity, age of 1 year, used to power medical device B, which has a medium level of usage (e.g., in a medium range defined by an upper threshold usage level and a lower threshold usage level), having a medium average battery temperature during charging (e.g., in a medium range defined by an upper threshold temperature and a lower threshold temperature), and having a medium rate of capacity decline (e.g., in a medium range defined by an upper threshold rate and a lower threshold rate). Battery R3 is indicated has having low usage (e.g., in a low range defined by an upper threshold usage level and a lower threshold usage level), 80% capacity, age of 1.5 years, used to power medical device C, which has a low level of usage (e.g., in a low range defined by an upper threshold usage level and a lower threshold usage level), having a medium average battery temperature during charging, and having a low rate of capacity decline (e.g., in a low range defined by an upper threshold rate and a lower threshold rate).

Based on factors including the above, the computing device(s) determines recommendations 1808 including to switch the medical devices for batteries R1 and R3 (e.g., for load balancing), and to reduce charging temperature for battery R1. If this action is taken, it is forecasted to increase the mutual remaining battery lifespan for all of batteries R1-R3 from 6 months to 2 years. Specifically, the estimated remaining life of battery R1 is projected to increase from 6 months to 2 years, and the estimated remaining life of battery R2 is projected to be unchanged at 3.5 years, but the estimated remaining life of battery R3 is projected to decrease from 3.5 years to 3 years. In particular, this may optimize battery usage for the following reasons. Battery R1 has the lowest capacity. However, its connected device has the highest usage level, which may correspond with a high battery power output, and battery R1 has a high average battery temperature at charging, both of which contribute to its very high rate of capacity decline from an already low capacity, and contribute to its low remaining life. With the recommended changes, the remaining life of battery R1 is projected to increase drastically, with minimal impact on the longer remaining life for batteries R2 and R3. In some embodiments, the recommendation 1708 may be displayed on a computer or handheld of a user, such as an administrator, and may offer the user an option related to implementing the recommended action, such as, e.g., an option to store a note or order to cause the batteries to be switched, or regarding reducing charging temperature.

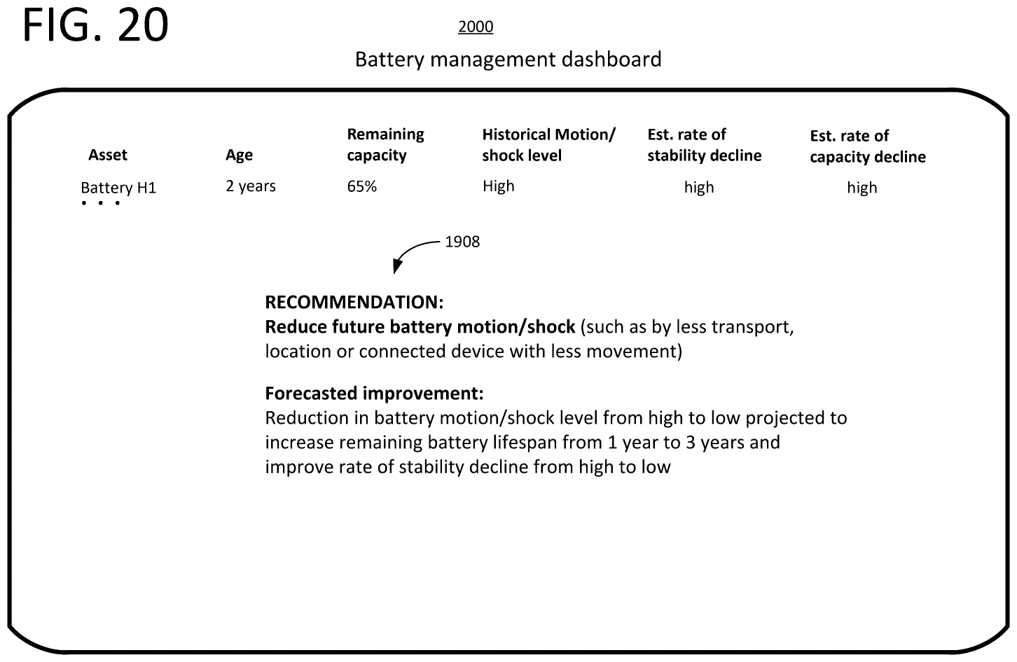

FIG. 20 provides an example of a dashboard 2000 that provides summary information for a group of batteries, including battery H1, which is determined and indicated as having an age of 2 years, a capacity of 65%, a high historical motion/shock level (e.g., in a high range defined by an upper threshold motion/shock level and a lower threshold motion/shock level), having a high rate of stability decline (e.g., in a high range defined by an upper threshold rate and a lower threshold rate) (e.g., based on risk of battery failure or specific error states occurring), and a high rate of capacity decline (e.g., in a high range defined by an upper threshold rate and a lower threshold rate). Based at least on these factors, it may be determined that excessive battery motion/shock is substantially contributing the battery's high rates of stability decline and capacity decline. Therefore, the computing device(s) determines a recommended action of reducing future motion or shock of battery H1, and provides example specific potential ways to achieve this, including less battery transport, or use with a connected device with less movement. A message is provided that reduction in battery motion/shock level from high to low (e.g., in a low range defined by an upper threshold motion/shock level and a lower threshold motion/shock level) is projected to increase remaining battery lifespan from 1 year to 3 years and improve the rate of stability decline from high to low (e.g., in a low range defined by an upper threshold rate and a lower threshold rate). In various examples, many factors, which may be reflected in historical battery health data, may lead to a recommended action for the purpose related to battery stability, including, among others, battery location, battery environment conditions, battery capacity, battery age, battery error states, battery impedance, battery power output, battery charging, and a connected device to be powered by the medical device battery. In some embodiments, the recommendation 1808 may be displayed on a computer or handheld of a user, such as an administrator, and may offer the user an option related to implementing the recommended action, such as an option to store a note or order regarding reducing motion/shock.

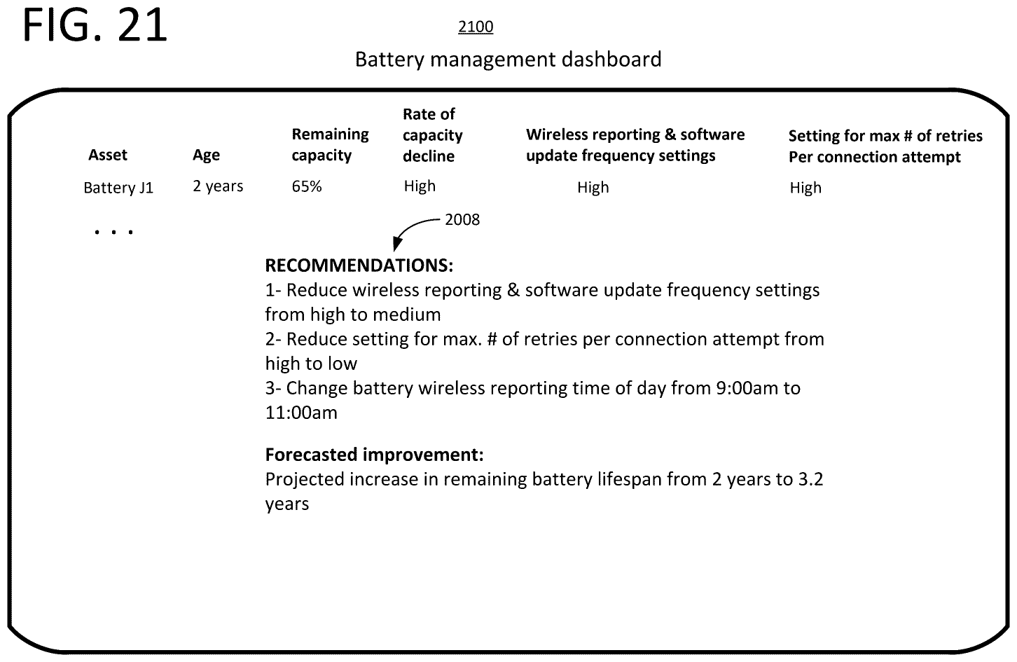

FIG. 21 provides an example of a dashboard 2100 that provides summary information for a group of batteries, including battery J1, which is determined and indicated as having an age of 2 years, a capacity of 65%, a high rate of capacity decline (e.g., in a high range defined by an upper threshold rate and a lower threshold rate), wireless reporting and software update frequency settings as high (e.g., in high ranges, each range defined by an upper threshold setting and a lower threshold setting) (e.g., where the wireless reporting could be initiated or transmitted from a battery or connected device), and the setting for maximum number of retries per connection attempt as high (e.g., in a high range defined by an upper setting and a lower setting). Based at least on these factors, it may be determined that the wireless transmission settings for battery J1 are leading to excessive power output for wireless transmission. As such, the computing device(s) determines a recommended actions 2008 of reducing the wireless reporting and software update frequency settings from high to medium (e.g., in medium ranges, each range defined by an upper threshold setting and a lower threshold setting), reduce the maximum number of retries per connection attempt setting from high to low (e.g., in a low range defined by an upper setting and a lower setting), and change a battery wireless reporting time of day from 9:00 am to 11:00 am (e.g., it may be forecasted, based on historical battery health data, that wireless signal will likely be higher at 11:00 am than at 9:00 am). In various examples, many factors, which may be reflected in historical battery health data, may lead to a recommended action relating to wireless communication settings or protocol, including, for example, among other things, wireless connection frequency, wireless connection attempt frequency, wireless reporting frequency, software update frequency, and wireless communication software protocol, wireless communication day or time related protocol battery location, wireless signal level, battery location, and wireless signal level (which may, e.g., be forecasted to vary depending on month, day of week, or time of day, for example). In some embodiments, the recommendation 1908 may be displayed on a computer or handheld of a user, such as an administrator, and may offer the user an option related to implementing the recommended action, such as an option to cause a remote computing device to transmit a command to implement to recommended changes regarding wireless reporting and software update frequencies, maximum retries per connection, and wireless reporting time.

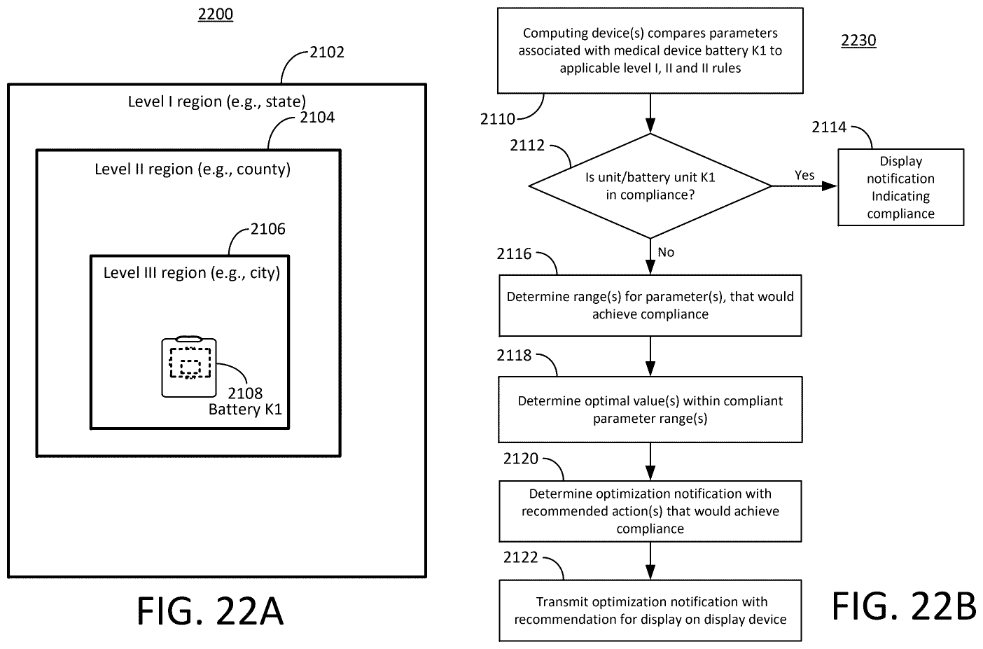

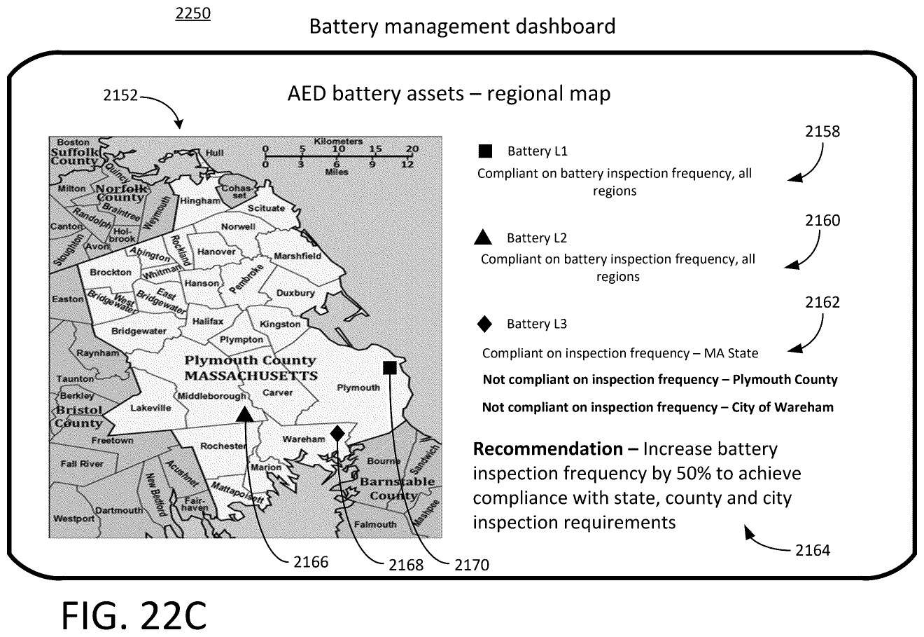

FIG. 22A, B and C include, in association with a medical device battery compliance engine, respectively, a block diagram 2200 illustrating an example associated regional structure, a flow diagram 2101 illustrating an example method, and an example compliance related dashboard 2150 including an associated map 2152. In some embodiments, recommended actions are associated with, or take into account, compliance with rules, such as federal, state and local governmental laws and regulations, associated with medical device batteries and/or connected devices, such as medical devices, e.g., defibrillators, including AEDs, power usage, data usage, permissions, licensing, reporting, inspection, recall, and error status, among other things. In addition, in some embodiments, compliance engines, and embodiments associated with compliance engines, relate to devices other than batteries, units, and connected devices of units. For example, some embodiments relate to locations, rules and recommendations relating to devices, such as medical devices (e.g., defibrillators or AEDs) (e.g., other than batteries, units and connected devices of units). For example, rules may specify requirements in connection with transmitting or receiving data (e.g., data or wireless connection limitations), device maintenance (e.g., registrations, frequency of battery, connected device, or other device maintenance activities or inspections), notification and data submission requirements (e.g., notifications required to be provided to governmental entities, regulatory entities, emergency medical services, medical or hospital entities), required device or device operation licensing, required recall activities, requirements relating to consumables associated with devices, requirements relating to provided data regarding location or site, and others.

Furthermore, in some instances, multiple entities, such as governmental entities, may each issue sets of rules, and/or multiple sets of rules may apply to a single location, such as a location of a medical device battery or connected device (or unit including a connected device and one or more medical device batteries) or other device or medical device. For example, applicable rules may be issued that are associated with each of multiple, overlapping regions, such as may be associated with a hierarchy of regional levels (or otherwise), e.g., zip code, state, county and city. Moreover, in some instances, each of multiple sets of rules may specify a different, though overlapping, range of compliant values (e.g., different, but overlapping, inspection frequency ranges).

In some embodiments, the location of the battery, unit or medical device (e.g., as may be detected by a location manager or GPS receiver thereof) is transmitted and input to a database (e.g., a medical device database) and used to determine the applicable rules for compliance. If the battery, unit or device is moved to a new location, then the new location may be detected, transmitted and input the database, to update the location. Based on the new location, the compliance engine may determine applicable rules for that location, which may be different than those of the previous location, and notifications or adjustments may be provided accordingly. For example, the required wireless reporting frequency may be once every two weeks at the earlier location, but once every week at the new location. Once moved to the new location, a notification may be provided, such as that the battery, unit or medical device is no longer in compliance with this requirement, and that the wireless reporting frequency should be changed from once every two weeks to once every week, or an automatic adjustment may be made to change the reporting frequency from once every two weeks to once per week.

Although many different regional or level types, structures, and numbers of levels are possible in different embodiments, as an example, some embodiments take into account rules associated with seven region types or levels, including, from highest level to lowest level, country, administrative area level 1, administrative area level 2, locality, sublocality level 1, sublocality level 2, and postal code. A specific region type associated with each level under country may depend on various factors, including the country. Examples of each level under country may include the following. For administrative area level 1, examples may include state, prefecture or region. For administrative area level 2, examples may include county, district, department or state. For locality, examples may include city or municipality. For sublocality level 1, examples may include subdivision, ward, arrondissement or district. For sublocality level 2, examples may include commune or subdistrict. For postal code, examples may include ZIP code, postal code or postal addressing code or, which, in Brazil, may be known as CEP.

In some embodiments, a compliance engine includes or takes into account compliance rules associated with connected devices of batteries or units, such as docks or defibrillators, such as AEDs, or devices, such as medical devices other than batteries or units (e.g., defibrillators or AEDs). In some instances, such requirements, even if not explicitly associated with batteries, may implicitly relate or extend to batteries-e.g., a requirement that an AED be kept in operative condition may effectively require that a battery (e.g., of a unit including an AED) be maintained with sufficient charge to support anticipated potential operation of the AED (e.g., to deliver defibrillation shocks). For example, in some embodiments, rules may relate to, for example, government or state rules (e.g., laws, statutes or regulations) requiring publicly accessible AEDs to have an associated prescription written by a local qualified medical professional, requiring AED owners/owning entities to have staff certified in AED operation, or requiring that all publicly accessible AEDs are reported to an emergency medical services (EMS) agency. As further example, requirements (of AEDs or other devices, such as batteries or units) may relate to, e.g., maintenance according to manufacturer's requirements, requirements that a device be provided at specific types of facilities, such as for-profit health or fitness centers, maintaining sufficient, and sufficiently trained, staff and management at associated facilities, inspections and maintenance of devices in operative condition, and others.

Given the foregoing, managing compliance issues can be complex. In some embodiments, recommended actions are determined and provided that relate to such compliance. For example, in some embodiments, a computing device(s), using a battery management and optimization program, uses obtained historical battery health data to assess compliance status with regard to multiple sets of rules. The computing device(s) may then determine recommendations to create or increase compliance, and/or to provide optimal values within compliant ranges, for example. For example, various rules may vary by state, county and city, such as regarding specifics of device accessibility (e.g., specified types of facilities, such as fitness centers, may be required to store an AED), maintenance (e.g., a device may be required to be maintained in a specified or useable condition—e.g., an AED being operational and/or an AED battery being kept sufficiently charged) and any required staffing (e.g., rules may specify a medical professional of specified qualifications be present in the city where the device, such as an AED, is kept), or device inspection frequency. If a device (e.g., a battery, unit or other medical device), for example, is moved from one state to another, then requirements between the state, county and city from which the device is moved (the origin location) to the state, county and city to which the device is moved (the destination location) may differ. For example, the destination location may have more strict standards regarding inspections (e.g., more frequent device inspects, or inspections associated with the destination state, county or local government), maintenance (e.g., the origin location may not require maintenance in accordance with manufacturer standards, while the destination location may require this), or medical staffing requirements (e.g., the origin location may have rules specifying that a medical professional be available in the city, while the destination location may have more strict rules requiring that the medical professional be a physician, or have certain certifications, or be familiar with certain local rules such as applicable local EMS protocols, which may not be required by the origin location).

As such, if a device is moved from the origin location to the destination location, these additional or stricter rules may need to be complied with, and actions may be recommended accordingly (e.g., increasing inspection frequency or types to ensure compliance, increasing or changing maintenance policies to ensure compliance, or increasing or changing staffing policies to ensure compliance). Conversely, if the destination location has less, or less strict rules, than actions may be recommended accordingly, as well (e.g., if the destination location has a less frequency inspection requirement, than an action could be recommended to reduce inspection frequency accordingly, etc.). Furthermore, in some embodiments, such actions, or related actions, may be automatically implemented (e.g., updating a database storing applicable policies, or, in some cases, automatically changing battery, unit, or other device parameters or aspects of usage, such as, e.g., increasing a wireless reporting frequency, if required by local rules, or increasing planned maintenance frequency, for example).

For example, in some embodiments, as shown in FIG. 22A, a medical device battery, or unit including a medical device battery K1 (which is shown as part of a unit including an AED, but, in other examples, may not be with a connected device, or may be with a different connected device), is located in multiple regions, each of which may issue rules. These regions may be hierarchical and range from larger to smaller, or otherwise. As shown, a largest, or level I, region 2102, is the U.S. state; a second largest, or level II, region 2104, is the county within the state; and a third largest, or level III, region 2106, is the city or town with the county. In some embodiments, historical battery health data is used in assessing compliance with rules relating to each of these levels, and recommendations are determined accordingly. Additionally, in some embodiments, one or more algorithms are used in, without requiring user input, periodically obtaining, updating, and storing data relating to rules from various entities, and relating to various levels, that may apply to locations at which medical device batteries, such as commonly owned medical device batteries, may be located. For example, this data may be stored in a medical device database, and may represent an updated library of such rules for a geographic region, for example. This library may then be used in analyzing compliance for one or more medical device batteries or medical devices (e.g., defibrillators or AEDs), and for providing information and optimization notifications, including recommended actions, to an administrative user, for example.

FIG. 22B provides an example method 2230 for determining an optimization notification including a recommended action associated with rule compliance. At step 2110, the computing device(s) compares parameters associated with one or more medical device batteries (e.g., battery K1) to multiple applicable sets of rules, such as level I, level II and level III rules, as described with reference to FIG. 21A.

At step 2112, based on the comparison, the computing device(s) queries whether battery K1 is in compliance with all applicable rules. If so, then, at step 2114, a notification is displayed, such as to an administrative user, indicating that battery K1 is in compliance with all applicable rules. If not, then, at step 2116, the computing device(s) determines a range for one or more parameters that would achieve compliance.

At step 2118, the computing device(s) determines one or more optimal values within compliance ranges. As one example of this, the computing device(s) may determine that the current wireless software update frequency setting for the battery is too low for compliance, and may determine a range of wireless software update frequencies that would achieve compliance with all applicable rules. At step 2120, the computing device determines an optimal value within the range. For example, the computing device(s) may determine that the optimal wireless software update frequency is the lowest frequency in that range, since that may be sufficient operationally and may require the least battery power output, thereby leading to maximum battery remaining lifespan. At step 2122, the computing device(s) transmits the optimization notification with the recommendation for display on a display device, such as of an administrative user.

FIG. 22C provides an example dashboard 2250 including a regional map 2152, specifically, of Plymouth County of the state of Massachusetts, displaying information relating to several batteries, such as batteries belonging to specified, user-defined group, including batteries L1, L2 and L3. In the example shown, icons are displayed on the map in positions that indicate the location of each battery. Specifically, battery L1 is indicated by a square shape icon 2170, battery L2 is indicated by a triangle shape icon 2166, and battery L3 is indicated by a diamond shape icon 2168. Displayed information 2158, 2160 includes that batteries L1 and L2 are compliant on inspection frequency rule requirements for all regions, but that battery L3 is not compliant on inspection frequency rule requirements for the larger Plymouth county region as well as the smaller City of Wareham region 2162. An optimization notification is determined and displayed that includes a recommended action 2164 to increase battery inspection frequency by 50% in order to achieve compliance with all state, county and city inspection requirements. For example, it may have been determined that increasing the battery inspection frequency by 50% achieves a frequency within all required, potentially overlapping ranges, or that increasing the battery inspection frequency by 50% achieves the lowest and potentially optimal frequency within a range of compliant values.

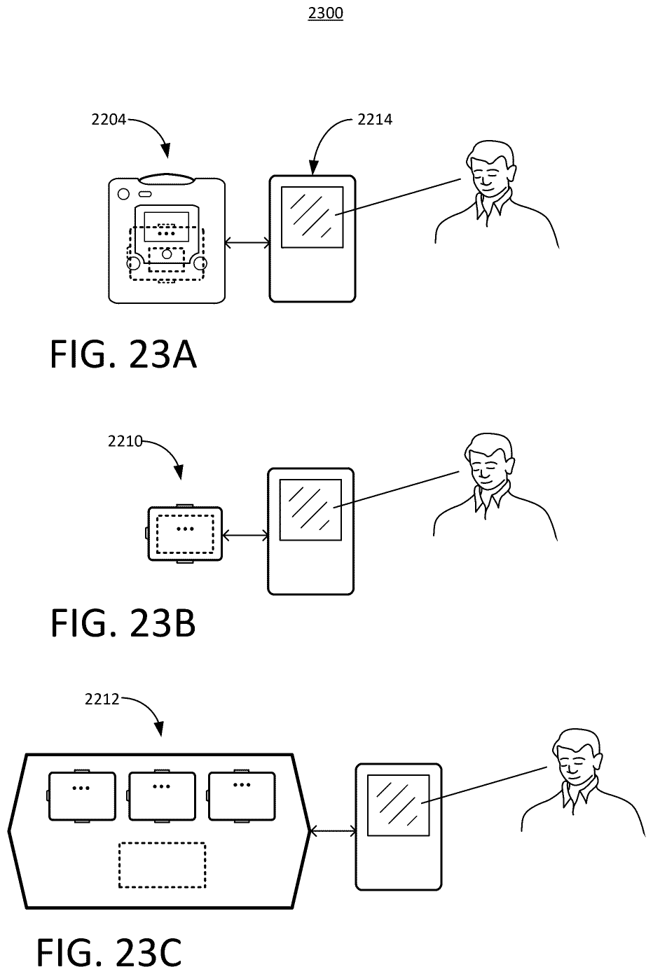

FIGS. 23A-C illustrate various examples of proximity based triggering of transmission and display (e.g., display device 2214) of historical battery health data from a medical device battery (e.g., battery 2210) or unit (e.g., units 2210, 2212). In some embodiments, medical device batteries, or connected devices of units, include hardware and/or software for local, proximity detection based wireless connection and communication, such as to allow an administrative user to obtain and display battery health data, which may include historical battery health data stored and transmitted by a medical device battery or unit. For example, in some embodiments, the battery and/or connected device includes one or more passive locator systems or components, such as RFID tags, systems or components, and/or one or more active locator systems or components, such as RTLS tags, systems or components, or GPS, Bluetooth or other low power systems or components. Passive tags, including RFID tags, may be powered by the receiving device, while active tags, such as RTLS tags, may be powered by the medical device battery itself. RTLS may an advantage by providing for more accurate, longer range tracking, but RFID may provide an advantage in not requiring power from the medical device battery. Furthermore, in some embodiments, a Wi-Fi positioning system may be used, or additionally, used, in locating batteries.

In some embodiments, when a suitable receiving display device becomes sufficiently proximate to a battery or connected device, wireless transmission of battery health data, which may include historical battery health data, may be initiated. For example, an administrative user, using the receiving device, may assess or track battery health or statues for each of a group batteries within various rooms of a building, hospital, warehouse, or other local area, and/or may store the data, which may be transmitted elsewhere, such as to a medical device database. In some embodiments, however, medical device batteries and connected devices may include ports, such as USB ports, to allow convenient wired connection of receiving display devices. Furthermore, in some embodiments, each battery may have a control, such as a physical or GUI-based button, that a user may press to initiate wired or wireless communication.

In some embodiments, for example, when exposed to FR energy from a handheld transmitter (or transceiver), an RFID tag of a battery causes a processor of the battery (or unit) to transition from a low power output sleep or standby mode to a higher power output wake mode in which the processor may cause historical battery usage data to be transmitted, using a wireless communication interface of the battery, to the handheld computing device (e.g., transmitter or transceiver, which may include a display), and/or, e.g., a computing device(s) at a central station. For example, in some embodiments, the processor may be programmed to periodically transition from a sleep mode to a wake mode for a period of time to perform periodic data transmissions before returning to sleep mode, such as to transmit historical battery health data. However, when awoken by RF energy from a transmitter, the battery may enter a transition to wake mode for a period of time in addition to such periodic transmission transitions. As such, in some embodiments, a user using a transmitter could cause a battery to wake up and transmit data, such as historical battery health data, to the handheld computing device a central station, for example.

In some embodiments, the user of the handheld device may also obtain various status information and/or recommendations, about a group of batteries, or selected or identified batteries within a group (e.g., registered batteries, batteries of a specific manufacturer or type, batteries with specific serial numbers, etc.).

For example, in a hospital setting, a user may be responsible for managing many batteries or units, such as 20, 30, 50 or more, potentially across a range of battery types, manufacturers, connected devices, etc. The user may be responsible for periodic battery maintenance activities, such as calibration and others, making tracking potentially very difficult. Additionally or alternatively, the user may be seeking to identify a small minority of batteries that require replacement. Furthermore, it may be important to identify batteries that have been stored for a long period of time. For example, a battery stored too long may require replacement or may go into a dormant state, with too low a voltage for normal charging or usage, for example. In some embodiments, rather than the user needing to check each battery by wired connection to each battery, at each battery's location, one at a time, the user, while in the hospital, using a handheld computing device, could cause each of a large group of batteries in the hospital to transmit battery health information, including historical battery health information, as well as battery usage optimization notifications/recommendations, such as to central station, or to the handheld computing device. This could include, for example, warnings regarding a batteries about to be stored too long, and a recommended action to charge them, for example. Such an arrangement may be used to greatly enhance and speed battery tracking and actions to optimize battery usage for a large group of batteries. Additionally, the user may obtain and display and store a variety of historical battery health data from each of the batteries, as well as notifications/recommendations, greatly enhancing tracking and maintenance efficiency. Furthermore, the user may be notified of the many batteries not in need of attention, thus saving large amounts of wasted time and effort.

FIG. 24 is an example environment 2400 including example wifi-enabled devices 2402a-c and a mifi device 2404. The wifi-enabled devices 2402a-c may include one or more medical device batteries or units as described herein. In the example, shown, the mifi device 2404 includes a memory 2416 including a battery communications/management program 2418, which represents programming or software used in implementing embodiments described herein.

As shown, the mifi device 2404 includes components/features including a cellular module 2408 (used in enabling cellular communications aspects), a wifi module 2410 (used in enabling wifi communication aspects), a processor 2412, a battery 2424 and a memory 2416 including the battery communications/management program 2418, and may also include a display/GUI 2406. In various embodiments, various of these features and components may be combined in various ways, or may include shared or overlapping aspects. In some embodiments, batteries and units, as described herein, may be wifi-enabled, and may be wirelessly connectable to the mifi device 2404 (e.g., via a location manager of the battery or unit). The mifi device 2404 may provide router functionality, and may be used in establishing wifi communication with (and, in some examples, between) the wifi-enabled devices 2402a-c, including the batteries and units. For example, the mifi device 2404 may be used in enabling connected batteries or units to transmit data, such as battery health data, via the Internet to one or more remote computing devices, and/or may enable the batteries or units 2404 to receive data from one or more other devices, such as one or more remote computing devices, via the Internet.

As described herein, batteries and units may periodically transmit battery-related information, which may include battery health information of various types. In some examples, this information may be used in generating summary data, or portions of summary data, and notifications/recommendations for battery usage optimization. As described, in some embodiments, batteries or units may wirelessly transmit battery health information via connection of the battery or unit to the Internet, such as without connection to a wifi network or connection to a mifi device 2404. However, in some embodiments, for example, the mifi device 2404 may be used in establishing a wifi connection for wifi-enabled batteries and units (and potentially other devices, such as medical devices), e.g., for batteries or units that are not cellular enabled, or as an alternative for batteries or units that are. The devices, including batteries or units, may connect to a wifi network, enabled by the mifi device 2404, which connection may be used, for example, in periodically wirelessly transmitting battery health information. In some embodiments, communications interfaces of batteries or units, as described herein, include hardware and/or software necessary for wifi connection, and enable connection to the mifi device 2404. Furthermore, in some embodiments, the mifi device 2404 may obtain, store and periodically transmit data or reports, such as to a remote computing device, such as may include historical, time-stamped or otherwise chronologically ordered logs of mifi activity and operational parameters. Furthermore, some embodiments, the mifi device may obtain, store and transmit historical battery health information obtained from batteries and units, such as to a remote computing device.

For example, in some examples, the mifi device 2404 may be used in a building (e.g., hospital), room, or other indoor or outdoor area in which multiple batteries or units are located, and may, for example, be used in enabling battery health data transmission from the devices to one or more remote computing devices in sufficient proximity with the mifi device 2404. In other examples, the mifi device 2404 may be used at the scene of treatment of a patient (e.g., at a hospital or indoor or outdoor scene of a patient health emergency), where the mifi device 2404 may be kept or carried, enabling communications for devices (which may include batteries or units) at the scene, such as may include, e.g., rescue, patient treatment and critical care communications.

For example, in some embodiments, the mifi device 2404 may enable wifi-enabled batteries, units or devices to periodically transmit historical battery healthy data to a remote computing device, which remote computing device may use the data in providing battery management dashboard displays, summary data relating to batteries or units, and battery usage optimization notifications/recommendations. Additionally, in some embodiments, the mifi device 2404 may be used in enabling batteries and units to receive transmissions from the remote computing device, such as to implement adjustments or changes to optimize battery usage (e.g., to adjust periodic battery health data transmission frequency or other conditions).

In some embodiments, the mifi device 2404 may have various user configurable features or parameters, which may, e.g., be set by a user via the display/GUI of the mifi device 2406 or at a separate or remote display/GUI wirelessly connected to the mifi device 2404.

In some embodiments, the mifi device 2404 may have features that enable it to provide information to batteries, units or other devices, or a remote computing device, in connection with battery management and battery usage optimization. For example, in some embodiments, the mifi device 2404 may include a location manager, such as GPS receiver. The mifi device may itself transmit location data relating to batteries or units with which is connects, such as if the batteries or units themselves may not have location managers, such as GPS receivers, or, even if they do, to spare the batteries or units the need to power them, for example, thus helping conserve battery charge level and reduce power output for the battery or unit. Additionally, in some embodiments, the mifi device 2404 itself may obtain, store and transmit reception level data, which may indicate cellular or wifi reception/signal levels (or, e.g., signal clarity, degree of interference, or other factors affecting ability to transmit or receive data). Furthermore, in some embodiments, the mifi device 2404 may store such information, and potentially other information, over time, and periodically transmit historical data including such information. To conserver or optimize power usage, for example, the mifi device may be programmed to transmit less often when signal is lower (and, e.g., more connection time or connection attempts may be required), or to not transmit until signal is higher. In some embodiments, the mifi device 2404 may itself receive and store battery health data from the batteries, units or other devices, and may transmit such data to a remote computing device, for example. Additionally, in some embodiments, the mifi device 2404, based on historical data relating to level of reception/signal at a location, may increase or decrease battery transmission frequencies, or other battery operational parameters or settings, such as to reduce the number of connections and/or failed connection attempts at low-signal locations.

Furthermore, in some embodiments, the mifi device 2404 may include functionality described in various embodiments with regard to a remote computing device, such as in using historical battery health data in determining and/or displaying battery usage optimization notifications/recommendations. For example, in some embodiments, the mifi device 2404 may be programmed to determine and display battery use optimization notifications/recommendations, such as, e.g., relating to increasing or reducing frequency of battery or unit periodic transmissions under certain conditions, such as reducing frequency if the battery is approaching a critically low level of charge, such as a threshold level of charge for providing anticipated potential care, such as, e.g., defibrillation shocks. In some embodiments, the mifi device 2404 may include user configurable aspects, such as to set specific parameters relating to certain aspects (e.g., specific rules or logic relating to when to increase or decrease frequency or content of battery, unit or other device periodic transmissions, or by how much, for example).

Furthermore, in some embodiments, the mifi device 2404 may include features to conserve its own battery 2424 power usage and charge, which, in some embodiments, may be user configurable. For example, in some embodiments, the mifi device 2404 may go into a low power consumption or sleep mode, turn off, or wake up to a regular power consumption mode, under specified conditions-e.g., relating to the mifi device 2404 (e.g., charge remaining on the mifi device battery), devices connected to it (e.g., medical devices, batteries or units), or environmental conditions (e.g., reception/signal level). For example, the sleep mode may include the cellular module 2408 being turned on, but the wifi module 2410 being turned off. For example, the mifi device 2414 may wake up at times when battery or unit transmissions are anticipated, or when communications relating to such transmissions, or the transmissions themselves, are initiated, and may go into a sleep mode for a period of time following activity, then turn off. Additionally, in some embodiments, the mifi device 2404 may select its mode (sleep, off, wake up) based on current or historical reception/signal level (e.g., go into sleep mode if reception is lower than usual, or go into wake up mode if reception/signal is higher than usual, or go into wake up mode on days or times when, historically, reception/signal is determined to be likely to be higher than usual, or go off when not needed to operate). Additionally, if the mifi device 2404 includes a location manager (e.g., GPS receiver), the location manager may only be activated when needed, to conserve mifi battery 2424 charge level or power output. Still further, in some embodiments, the mifi device 2404 may be programmed to not transmit, even for otherwise scheduled or due transmissions, unless sufficient reception/signal is present, such as if the wifi senses that insufficient reception/signal is present and/or if the mifi is presently located in an area that is known or anticipated to have insufficient reception/signal. In some embodiments, the mifi may include a selectable mode of operation that includes that features. Furthermore, in some embodiments, battery usage optimization, conservation and/or load balancing may be implemented that takes into account or analyzes factors relating to the batteries of both the mifi device 2404 and one or more connected devices (e.g., batteries or units), and determines and implements settings or adjustments for such optimization, for example, on the mifi device 2404 and the one or more connected devices, e.g., to shift power usage (e.g., for location detection or wireless reporting) more to the device that is not low on battery power or approaching a low battery power threshold.

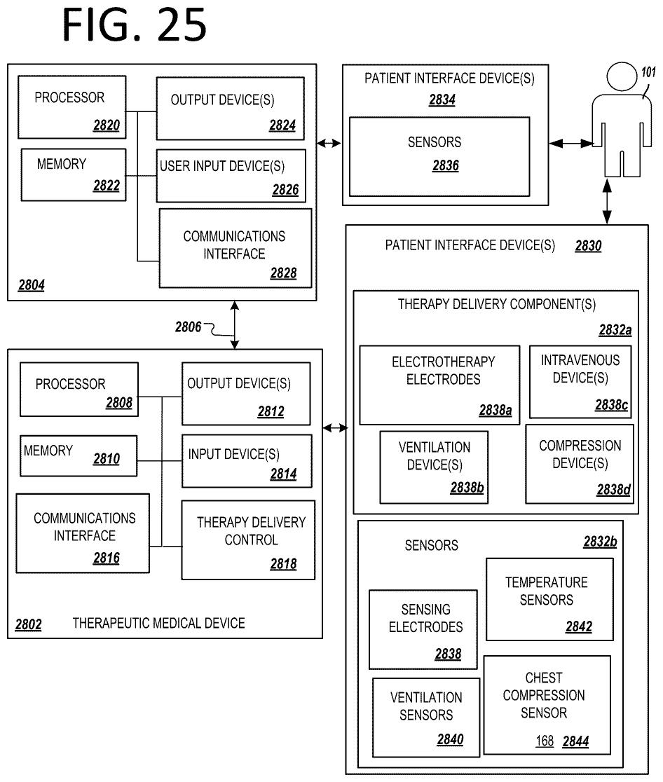

FIG. 25 illustrates an example of components of various devices described with reference to prior figures. The components 2808, 2810, 2812, 2814, 2816, and 2818 are communicatively coupled (directly and/or indirectly) to each other for bi-directional communication. Similarly, the components 2820, 2822, 2824, 2826, and 2828 are communicatively coupled (directly and/or indirectly) to each other for bi-directional communication.

In some implementations, the components 2808, 2810, 2816, and/or 2818 of the therapeutic medical device or medical device 2802 may be combined into one or more discrete components and components 2816 and/or 2818 may be part of the processor 2808. The processor 2808 and the memory 2810 may include and/or be coupled to associated circuitry in order to perform the functions described herein. Additionally, the components 2820, 2822, and 2828 of companion device 2804 may be combined into one or more discrete components and component 2828 may be part of the processor 2820. The processor 2820 and the memory 2822 may include and/or be coupled to associated circuitry in order to perform the functions described herein, including those associated with a battery management and optimization program.

In some implementations, the therapeutic medical device 2802 may include the therapy delivery control module 2818. For example, the therapy delivery control module 2818 may be an electrotherapy delivery circuit that includes one or more high-voltage capacitors configured to store electrical energy for a pacing pulse or a defibrillating pulse. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 2818 may be a compression device electro-mechanical controller configured to control a mechanical compression device. As a further example, the therapy delivery control module 2818 may be an electro-mechanical controller configured to control drug delivery, temperature management, ventilation, and/or other type of therapy delivery.

The therapeutic medical device 2802 may incorporate and/or be configured to couple to one or more patient interface devices 2830 and patient interface devices that may be coupled with a patient 2849. The patient interface devices 2830 may include one or more therapy delivery component(s) 2832a and one or more sensor(s) 2832b. Similarly, the companion device 2804 may be adapted for medical use and may incorporate and/or be configured to couple to one or more patient interface device(s) 2834. The patient interface device(s) 2834 may include one or more sensors 2836. The sensor(s) 2836 may be substantially as described herein with regard to the sensor(s) 2832b.

The sensor(s) 2832b and 2836 may include sensing electrodes (e.g., the sensing electrodes 2838), ventilation and/or respiration sensors (e.g., the ventilation and/or respiration sensors 2830), temperature sensors (e.g., the temperature sensor 2842), chest compression sensors (e.g., the chest compression sensor 2844), etc. In some implementations, the information obtained from the sensors 2832b and 2836 can be used to generate information displayed at the therapeutic medical device 2802 and simultaneously at the display views at companion device 2804 and described above. In one example, the sensing electrodes 2838 may include cardiac sensing electrodes. The cardiac sensing electrodes may be conductive and/or capacitive electrodes configured to measure changes in a patient's electrophysiology to measure the patient's ECG information. The sensing electrodes 2838 may further measure the transthoracic impedance and/or a heart rate of the patient. The ventilation and/or respiration sensors 2830 may include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, impedance sensors, and combinations thereof. The temperature sensors 2842 may include an infrared thermometer, a contact thermometer, a remote thermometer, a liquid crystal thermometer, a thermocouple, a thermistor, etc. and may measure patient temperature internally and/or externally. The chest compression sensor 2844 may include one or more motion sensors including, for example, one or more accelerometers, one or more force sensors, one or more magnetic sensors, one or more velocity sensors, one or more displacement sensors, etc. The chest compression sensor 2844 may provide one or more signals indicative of the chest motion to the therapeutic medical device 2802 via a wired and/or wireless connection. The chest compression sensor 2844 may be, for example, but not limited to, a compression puck, a smart-phone, a hand-held device, a wearable device, etc. The chest compression sensor 2844 may be configured to detect chest motion imparted by a rescuer and/or an automated chest compression device (e.g., a belt system, a piston system, etc.). The chest compression sensor 2844 may provide signals indicative of chest compression data including displacement data, velocity data, release velocity data, acceleration data, force data, compression rate data, dwell time data, hold time data, blood flow data, blood pressure data, etc. In an implementation, the defibrillation and/or pacing electrodes may include or be configured to couple to the chest compression sensor 2844.

In various implementations, the sensors 2832b and 2836 may include one or more sensor devices configured to provide sensor data that includes, for example, but not limited to ECG, blood pressure, heart rate, respiration rate, heart sounds, lung sounds, respiration sounds, end tidal CO₂, saturation of muscle oxygen (SMO₂), oxygen saturation (e.g., SpO₂ and/or PaO₂), cerebral blood flow, point of care laboratory measurements (e.g., lactate, glucose, etc.), temperature, electroencephalogram (EEG) signals, brain oxygen level, tissue pH, tissue fluid levels, images and/or videos via ultrasound, laryngoscopy, and/or other medical imaging techniques, near-infrared spectroscopy, pneumography, cardiography, and/or patient movement. Images and/or videos may be two-dimensional or three-dimensional, such a various forms of ultrasound imaging.

The one or more therapy delivery components 2832a may include electrotherapy electrodes (e.g., the electrotherapy electrodes 2838a), ventilation device(s) (e.g., the ventilation devices 2838b), intravenous device(s) (e.g., the intravenous devices 2838c), compression device(s) (e.g., the compression devices 2838d), etc. For example, the electrotherapy electrodes 2838a may include defibrillation electrodes, pacing electrodes, and combinations thereof. The ventilation devices 2838b may include a tube, a mask, an abdominal and/or chest compressor (e.g., a belt, a cuirass, etc.), etc. and combinations thereof. The intravenous devices 2838c may include drug delivery devices, fluid delivery devices, and combinations thereof. The compression devices 2838d may include mechanical compression devices such as abdominal compressors, chest compressors, belts, pistons, and combinations thereof. In various implementation, the therapy delivery component(s) 2832a may be configured to provide sensor data and/or be coupled to and/or incorporate sensors. For example, the electrotherapy electrodes 2838a may provide sensor data such as transthoracic impedance, ECG, heart rate, etc. Further the electrotherapy electrodes 2838a may include and or be coupled to a chest compression sensor. As another example, the ventilation devices 2838b may be coupled to and/or incorporate flow sensors, gas species sensors (e.g., oxygen sensor, carbon dioxide sensor, etc.), etc. As a further example, the intravenous devices 2838c may be coupled to and/or incorporate temperature sensors, flow sensors, blood pressure sensors, etc. As yet another example, the compression devices 2838d may be coupled to and/or incorporate chest compression sensors, patient position sensors, etc. The therapy delivery control modules 2818 may be configured to couple to and control the therapy delivery component(s) 2832a, respectively.

The one or more sensor(s) 2832b and 2836 and/or the therapy delivery component(s) 2832a may provide sensor data. The patient data provided at the display screens of the therapeutic medical device 2802 and companion device 2804 may display the sensor data. For example, the therapeutic medical device 2802 may process signals received from the sensor(s) 2832b and/or the therapy delivery component(s) 2832a to determine the sensor data. Similarly, the companion device 2804 may process signals received from the sensor(s) 2836 and/or sensor data from the sensors 2832b received via the therapeutic medical device 2802 to determine the sensor data.

The description set forth herein in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

As used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “substantially”, “about,” “proximate,” “minor variation,” and similar terms generally refer to ranges that include the identified value within a margin of 20%, 10%, 5%, or less than 5%, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

In some instances, variations of a term may be utilized that may refer to the same or similar concepts, and certain terms may have meanings that are informed by a particular context. Generally, sending, receiving, or transmitting of data may include by wired and/or wireless connection, and/or within one or more wired or wireless networks. Furthermore, sending from a first entity to a second entity, or to be received by the second entity, can include sending from the first entity to the second entity, or to be received by the second entity, directly from the first entity to the second entity, or indirectly via one or more intermediary entities.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A medical device battery management system for managing a group of commonly owned medical device batteries, comprising:
a plurality of medical device batteries, each comprising a processor, a memory and a wireless communication interface, and each configured to write battery health data to the memory and periodically transmit historical battery health data comprising past information relating to battery usage;
a medical device database configured to receive and store the historical battery health data for the plurality of medical device batteries; and
at least one remote computing device, communicatively coupled with the plurality of medical device batteries and the medical device database, the at least one remote computing device comprising at least one processor and at least one memory, the at least one remote computing device configured to:
receive the historical battery health data from the plurality of medical device batteries,
update the medical device database with the received historical battery health data,
analyze the historical battery health data to (i) identify one or more medical device batteries for which an optimization notification can be provided to recommend an action to optimize battery usage, and to (ii) determine the optimization notification,
receive a request from an administrative user relating to a user-defined grouping of medical device batteries to access at least the historical battery health data associated with the user-defined grouping of medical device batteries, and
generate and transmit, for display on a display device, summary data relating to the user-defined grouping of medical device batteries, and the determined optimization notification to recommend the action to optimize battery usage.

2. The system of claim 1, wherein the battery health data includes data relating to at least one of: battery condition, battery status, battery usage, battery error states, battery type, battery manufacturer, battery manufacture date, battery serial number, battery specification information, battery age, battery location, connected device, clinical event data associated with the connected device, battery temperature, battery environment temperature, battery humidity, battery environment humidity, battery motion, battery capacity, battery design capacity, battery remaining capacity, battery power output, battery remaining operating time, battery alarms, battery remaining capacity alarm, battery remaining operating time alarm, battery mode, battery stability, battery reliability, cycle life, and battery calibration.

3. The system of claim 1, wherein the battery health data includes data relating to at least one of: battery wireless communication, battery periodic reporting, and battery periodic software updating.

4. The system of claim 1, wherein the battery health data includes data relating to at least one or: battery charging, battery charging rate, battery absolute state of charge, battery relative state of charge, depth of discharge, battery full depth of discharges.

5. The system of claim 1, wherein the battery health data includes data relating to at least one of: minimum lifetime battery pack voltage, maximum lifetime battery pack voltage, minimum lifetime battery pack temperature during discharge, maximum lifetime battery pack temperature during discharge, maximum lifetime battery pack temperature during charge, maximum lifetime current during charge, maximum lifetime current during discharge, total discharge amp-hours throughput, total charge amp-hours throughput, energy density, specific energy, nominal capacity, nominal energy, terminal voltage, open-circuit voltage, cut-off voltage, nominal voltage, charge voltage, float voltage, discharge current, C-rate, recommended charge current, specific energy, energy density, specific power, and power density.

6. The system of claim 2, wherein the battery error states relate to at least one of: battery physical structure, battery configuration, battery hardware, battery software, battery sensors, battery wireless communication interface, battery ports, battery location manager, battery circuit boards, battery RTLS tags, battery RFID tags, battery connected device, a battery voltage fault, a battery pack voltage fault, an under voltage fault, an over voltage fault, a capacity fault, an absolute capacity below limit fault, a temperature fault, an under temperature fault, an over temperature fault, a current fault, a fault of the processor, a fault of the memory, a software fault, a PCB fault, an initial PCB power-on fault, a loss of power to PCB fault, an electronic circuit fault, a computer chip fault, a power-off fault, a reset fault, a clock frequency fault, a watchdog timer reset fault, an alarm fault, a cyclic redundancy check fault, a flash cyclic redundancy check fault, an integrated circuit power supply pins fault, a battery fuel gauge fault, a fuel gauge communication fault, a fuel gauge safety error fault, a VCC supply brownout, a CPU fault, a blown fuse fault, a sensor fault, an initial reconditioning charge cycle required fault, a charge protocol fault, a charge too long fault, a stuck button fault, a call stack overflow fault, a call stack underflow fault, an invalid configuration fault, a software initiated reset fault, a serial flash error fault, an invalid cyclic redundancy check on flash data fault, a microcontroller RAM fault, a battery pack disabled fault, a charge protocol fault, and a software interrupt fault.

7. The system of claim 1, wherein analyzing the historical battery health data comprises:
generating one or more data sets comprising chronologically ordered information relating to each of a plurality of battery parameters over a historical period of time, and
analyze the generated data sets in determining the action to be recommended to optimize battery usage.

8. The system of claim 7, wherein analyzing the historical battery health data comprises:
determining multiple potential actions, each of which is algorithmically forecasted to improve battery usage,
analyzing, and determining a score for, each of the multiple potential actions, wherein the score provides a measure of magnitude of improvement in battery usage forecasted to occur as a result of the potential action, and
determine the action to be recommended to optimize battery usage based at least in part on the determined scores.

9. The system of claim 1, wherein the determined optimization notification comprises at least one of: an alert, an alarm, a warning, a message, a message recommending a remedial action, and a message recommending an enhancing action.

10. The system of claim 1, wherein the at least one remote computing device is configured to implement the recommended action to optimize battery usage upon a user selection to do so, and wherein the optimization notification comprises an improvement in battery usage forecasted to result from implementation of the recommended action.

11. The system of claim 1, wherein each of the plurality of medical device batteries comprises at least one of: at least one motion sensor configured to detect motion of the medical device battery over a period of time, at least one battery temperature sensor configured to detect a temperature of the medical device battery over the period of time, at least one battery environment temperature sensor configured to detect a temperature of an environment of the medical device battery over the period of time, at least one battery humidity sensor configured to detect a humidity level of the medical device battery over the period of time, at least one battery environment humidity sensor configured to detect a humidity level of the environment of the medical device battery over the period of time, and at least one port configured for wired connection to at least one computing or display device.

12. The system of claim 1, wherein each of the plurality of medical device batteries comprises a location manager configured to obtain battery location data relating to the medical device battery, wherein the user-defined grouping is based on battery location.

13. The system of claim 12, wherein the at least one computing device is configured to determine the recommended action based at least in part on compliance criteria relating to compliance with battery related, location based legal or regulatory requirements associated with at least one of: devices, data usage, permissions, licensing, reporting, inspection, recall, and error status.

14. The system of claim 1, wherein each of the plurality of medical device batteries is configured to wirelessly transmit the historical battery health data at least one of: automatically according to a specified frequency or protocol, upon a user selection via a physical interface feature or graphical user interface of the battery unit, and upon detection of sufficient proximity with a receiving device for the historical battery health data.

15. The system of claim 1, wherein the recommended action comprises at least one of: switching locations between at least two medical device batteries, and switching connected devices to be powered by each of the at least two medical device batteries, and wherein determining the recommended action comprises utilizing at least a portion of the past information relating to at three of: battery location, battery environment conditions, battery capacity, battery age, battery error states, battery impedance, battery power output, battery charging, and a connected device to be powered by the medical device battery.

16. The system of claim 1, wherein the optimization notification relates to a change of storage location or a change of one or more storage conditions relating to at least one of the plurality of medical device batteries.

17. The system of claim 1, wherein each of the plurality of medical device batteries comprises a location manager configured to obtain battery location data relating to the medical device battery, wherein the recommended action comprises changing a location of a medical device battery in relation to at least one of: geographic location and in-building related location, and wherein determining the recommended action comprises utilizing at least a portion of the past information relating to at three of: battery location, battery wireless communication settings or protocol, battery motion or shock, battery environment conditions, battery capacity, battery age, battery error states, battery impedance, battery power output, battery charging, a connected device to be powered by the medical device battery.

18. The system of claim 1, wherein the recommended action comprises replacing a medical device battery, and wherein determining the recommended action comprises utilizing at least a portion of the past information relating to at three of: battery capacity, battery age, battery error states, battery impedance, battery power output, battery charging, and a connected device to be powered by the medical device battery.

19. The system of claim 1, wherein the recommended action comprises changing future battery charging conditions, relative to past battery charging conditions, the battery charging conditions comprising at least three of: battery charge levels during charging, battery temperature during charging, battery environment temperature during charging, battery humidity during charging, battery environment humidity during charging, battery motion or shock during charging, and wherein determining the recommended action comprises utilizing at least a portion of the past information relating to at three of: battery location, battery charge levels, battery temperature, battery environment temperature, battery humidity, battery environment humidity, battery capacity, battery motion or shock, battery capacity, battery impedance, and battery power output.

20. The system of claim 1, wherein the recommended action comprises reducing battery motion or shock, and wherein determining the recommended action comprises utilizing at least a portion of the past information relating to at three of: battery location, battery motion, battery shock, battery transport, battery impedance, battery error states, battery voltage, battery capacity.

21. The system of claim 1, wherein the recommended action comprises changing a wireless communication related setting or protocol, the wireless communication related setting or protocol relating to at least one of: wireless connection frequency, wireless connection attempt frequency, wireless reporting frequency, software update frequency, wireless communication software protocol, and wireless communication day or time related protocol, and wherein determining the recommended action comprises utilizing at least a portion of the past information relating to at three of: wireless connection frequency, wireless connection attempt frequency, wireless reporting frequency, software update frequency, and wireless communication software protocol, wireless communication day or time related protocol battery location, wireless signal level, battery location, and wireless signal level.

22. The system of claim 1, comprising a mifi device configured to connect to at least one of the plurality of medical device batteries, and wherein the at least one of the plurality of medical device batteries is configured to connect to the mifi device and to use the connection with the mifi device in transmitting the historical battery health data to the at least one remote computing device.

23-92. (canceled)
